SEQUENCE LISTING (64 sequences)

SEQ ID NO: 1 - protein, 11 aa, Artificial Sequence
  Synthetic construct
  MISC_FEATURE (2)..(2): The amino acid in position 2 of the conserved motif is any amino acid.
  MISC_FEATURE (6)..(6): The amino acid in position 6 of the conserved motif is isoleucine or leucine.
  MISC_FEATURE (9)..(9): The amino acid in position 9 of the conserved motif is any amino acid.
YXWWYXRRXYG

SEQ ID NO: 2 - protein, 27 aa, Artificial Sequence
  Synthetic construct
MKKPLGKIVASTALLISVAFSSSIASA

SEQ ID NO: 3 - protein, 8 aa, Artificial Sequence
  Synthetic construct
HHHHHHPR

SEQ ID NO: 4 - DNA, 1221 bp, Pseudoalteromonas tetraodonis
  CDS (1)..(1218)
  sig_peptide (1)..(72)
  mat_peptide (73)..(1218)
atggaaattagaaaactactaggcatttctgcctctctattactcagttcaattagcttagaggcattagcgagcaacgttaccattaattttaatacgcaatatcaacaaattgatgggttcggtggtatgaatgcacccggctggattaatgatttaacgcctgcacaggcaacaaaagcatttggtacgggtaatggtgaaatgggcttatctatcatgcgtatgcgcatagcacctgattctaatcagtggtacaaacaggtacctacagcaaaaattgctaaatcatatggtgcaaaactattggcttctccgtggtctccgccagcgtatatgaaaagcaataataatctaaataacggaggtaaattagaaaaaacacattattggggttataccaatcatttaatggacttcaccaattatatggcaagccaaggtgcatcggtttatgctttatcgctgcaaaatgagccggactggcatcctgagtatgagtcttgtgactggtcggcatcagattttgtcaattacttgaatgaccaaggttggcgactcgaccctgcgcttaaaattcttgctcctgaatcattaggatttaacaaagctttatcagatccaattttgaataattctgtggcaaataattatgtcgatatcattggtggtcatttatatggcgtatctccatcaaattacccactggcattgcaaaaaggtaaaaagttatggatgacagagcattataccgataatgaagacggtaataactggaatgcatcaattgatgtagggttggaactacatcaaagcatggtatcaaattatagtgcttatatttggtggtatatacgccgcagctacggattaatgagtgaagacggaaacgtaagcaaacgtggttatattatggctcagttttctaaatatattcgccctggttacacccgtattggcgccacagaaatgcctgaaaacaacgtatatgttactgcttacaaaaataacgcaggtaagcttgtgattgttgttgttaataaaagtggctcacctaaggcactcgattttactttgcaaaatggcacagttaacacactgacaaaatactctacatctgcatctatgaatatggaataccgaggaaaatcgacggtaagtaataatcgtttttctgcgtatgccgatgcgtgggcagtgcaaacatttgtatctaattaa

SEQ ID NO: 5 - protein, 406 aa, Pseudoalteromonas tetraodonis
MEIRKLLGISASLLLSSISLEALASNVTINFNTQYQQIDGFGGMNAPGWINDLTPAQATKAFGTGNGEMGLSIMRMRIAPDSNQWYKQVPTAKIAKSYGAKLLASPWSPPAYMKSNNNLNNGGKLEKTHYWGYTNHLMDFTNYMASQGASVYALSLQNEPDWHPEYESCDWSASDFVNYLNDQGWRLDPALKILAPESLGFNKALSDPILNNSVANNYVDIIGGHLYGVSPSNYPLALQKGKKLWMTEHYTDNEDGNNWNASIDVGLELHQSMVSNYSAYIWWYIRRSYGLMSEDGNVSKRGYIMAQFSKYIRPGYTRIGATEMPENNVYVTAYKNNAGKLVIVVVNKSGSPKALDFTLQNGTVNTLTKYSTSASMNMEYRGKSTVSNNRFSAYADAWAVQTFVSN

SEQ ID NO: 6 - protein, 382 aa, Pseudoalteromonas tetraodonis
  mat_peptide (1)..(382)
SNVTINFNTQYQQIDGFGGMNAPGWINDLTPAQATKAFGTGNGEMGLSIMRMRIAPDSNQWYKQVPTAKIAKSYGAKLLASPWSPPAYMKSNNNLNNGGKLEKTHYWGYTNHLMDFTNYMASQGASVYALSLQNEPDWHPEYESCDWSASDFVNYLNDQGWRLDPALKILAPESLGFNKALSDPILNNSVANNYVDIIGGHLYGVSPSNYPLALQKGKKLWMTEHYTDNEDGNNWNASIDVGLELHQSMVSNYSAYIWWYIRRSYGLMSEDGNVSKRGYIMAQFSKYIRPGYTRIGATEMPENNVYVTAYKNNAGKLVIVVVNKSGSPKALDFTLQNGTVNTLTKYSTSASMNMEYRGKSTVSNNRFSAYADAWAVQTFVSN

SEQ ID NO: 7 - DNA, 1254 bp, Artificial Sequence
  Synthetic Construct
  CDS (1)..(1251)
  sig_peptide (1)..(81)
  mat_peptide (82)..(1251)
atgaagaaaccgttggggaaaattgtcgcaagcaccgcactactcatttctgttgcttttagttcatcgatagcatcagcacatcatcatcaccatcatcctaggagcaacgttaccattaattttaatacgcaatatcaacaaattgatgggttcggtggtatgaatgcacccggctggattaatgatttaacgcctgcacaggcaacaaaagcatttggtacgggtaatggtgaaatgggcttatctatcatgcgtatgcgcatagcacctgattctaatcagtggtacaaacaggtacctacagcaaaaattgctaaatcatatggtgcaaaactattggcttctccgtggtctccgccagcgtatatgaaaagcaataataatctaaataacggaggtaaattagaaaaaacacattattggggttataccaatcatttaatggacttcaccaattatatggcaagccaaggtgcatcggtttatgctttatcgctgcaaaatgagccggactggcatcctgagtatgagtcttgtgactggtcggcatcagattttgtcaattacttgaatgaccaaggttggcgactcgaccctgcgcttaaaattcttgctcctgaatcattaggatttaacaaagctttatcagatccaattttgaataattctgtggcaaataattatgtcgatatcattggtggtcatttatatggcgtatctccatcaaattacccactggcattgcaaaaaggtaaaaagttatggatgacagagcattataccgataatgaagacggtaataactggaatgcatcaattgatgtagggttggaactacatcaaagcatggtatcaaattatagtgcttatatttggtggtatatacgccgcagctacggattaatgagtgaagacggaaacgtaagcaaacgtggttatattatggctcagttttctaaatatattcgccctggttacacccgtattggcgccacagaaatgcctgaaaacaacgtatatgttactgcttacaaaaataacgcaggtaagcttgtgattgttgttgttaataaaagtggctcacctaaggcactcgattttactttgcaaaatggcacagttaacacactgacaaaatactctacatctgcatctatgaatatggaataccgaggaaaatcgacggtaagtaataatcgtttttctgcgtatgccgatgcgtgggcagtgcaaacatttgtatctaattaa

SEQ ID NO: 8 - protein, 417 aa, Artificial Sequence
  Synthetic Construct
MKKPLGKIVASTALLISVAFSSSIASAHHHHHHPRSNVTINFNTQYQQIDGFGGMNAPGWINDLTPAQATKAFGTGNGEMGLSIMRMRIAPDSNQWYKQVPTAKIAKSYGAKLLASPWSPPAYMKSNNNLNNGGKLEKTHYWGYTNHLMDFTNYMASQGASVYALSLQNEPDWHPEYESCDWSASDFVNYLNDQGWRLDPALKILAPESLGFNKALSDPILNNSVANNYVDIIGGHLYGVSPSNYPLALQKGKKLWMTEHYTDNEDGNNWNASIDVGLELHQSMVSNYSAYIWWYIRRSYGLMSEDGNVSKRGYIMAQFSKYIRPGYTRIGATEMPENNVYVTAYKNNAGKLVIVVVNKSGSPKALDFTLQNGTVNTLTKYSTSASMNMEYRGKSTVSNNRFSAYADAWAVQTFVSN

SEQ ID NO: 9 - protein, 390 aa, Artificial Sequence
  Synthetic construct
  mat_peptide (1)..(390)
HHHHHHPRSNVTINFNTQYQQIDGFGGMNAPGWINDLTPAQATKAFGTGNGEMGLSIMRMRIAPDSNQWYKQVPTAKIAKSYGAKLLASPWSPPAYMKSNNNLNNGGKLEKTHYWGYTNHLMDFTNYMASQGASVYALSLQNEPDWHPEYESCDWSASDFVNYLNDQGWRLDPALKILAPESLGFNKALSDPILNNSVANNYVDIIGGHLYGVSPSNYPLALQKGKKLWMTEHYTDNEDGNNWNASIDVGLELHQSMVSNYSAYIWWYIRRSYGLMSEDGNVSKRGYIMAQFSKYIRPGYTRIGATEMPENNVYVTAYKNNAGKLVIVVVNKSGSPKALDFTLQNGTVNTLTKYSTSASMNMEYRGKSTVSNNRFSAYADAWAVQTFVSN

SEQ ID NO: 10 - DNA, 1266 bp, Paenibacillus sp.
  CDS (1)..(1263)
  sig_peptide (1)..(90)
  mat_peptide (91)..(1263)
atgaatttacggtttaagaaatccatatacgcatgtttggcccttgtcatggcattgtccatcctgggggcacctggaaaaattgccgcagccagcgacgccatcgtaaatatttcggcggaaaaacaagtgattcgtggtttcggggggatcaaccatccggtctggatcggggatttgacggcggcacaaagagaaaccgccttcgggaacggaaataaccaactgggtttctcgattttgagaatctatgtacacgaagaccgaaatcaatggtaccgggaagtggaaacggccaaacgagcgattgccctcggagccatcgtattcgcatcgccatggaatccgcccgccgacatggtcgagaccttcaaccgaaacggcgatccgtcggcaaagcggctccgttatgacaagtatgccgcgtatgcccagcatcttaacgacttcgtgacgtatatgagaaacaacggcgtgaatctgtatgcgatttccgtccagaacgagcccgattatgcccatgactggacgtggtggacgccgcaggaaatgcttcgtttcatgaaagaaaatgccggatcgattaatacgagagtgatcgcgccggaatcgttccagtatctgaaaaatatgtcggacccgatactaaacgatccgcaggcgctagccaatatggatattcttggcgctcatctgtacgggacccaggtcagcaatttcgcttatcctttgttcaaacaaaaaggagcgggcaaagatctctggatgacggaggtatactatccgaacagtgacaacaactcggcggatcgttggcccgaagccctggatgtgtcctaccatatccacaatgcgatggtagagggagattttcaagcttacgtatggtggtatatccgcagatcgtacggtccgatgaaagaggacggcacgatcagcaaacgcggttacgctatggcgcatttctccaaattcgtccgtcccgggtatgtccgggttgaagcgacgaaaaatccggaaacgaacgtttatgtatccgcctataaaggtaataagaagcttgttatcgttgccgtcaacaaaaataactccggggtaaatcagaactttgtcctgcccaatgcatcggtttcgaaaatctccagatggatcactagcggcagcagcaatcttcagccgggaacggagcttacgatgacgggtggaaatttctgggctcatctcccggctcaaagcgtgaccacctttgtcgctgatcttggctaa

SEQ ID NO: 11 - protein, 421 aa, Paenibacillus sp.
MNLRFKKSIYACLALVMALSILGAPGKIAAASDAIVNISAEKQVIRGFGGINHPVWIGDLTAAQRETAFGNGNNQLGFSILRIYVHEDRNQWYREVETAKRAIALGAIVFASPWNPPADMVETFNRNGDPSAKRLRYDKYAAYAQHLNDFVTYMRNNGVNLYAISVQNEPDYAHDWTWWTPQEMLRFMKENAGSINTRVIAPESFQYLKNMSDPILNDPQALANMDILGAHLYGTQVSNFAYPLFKQKGAGKDLWMTEVYYPNSDNNSADRWPEALDVSYHIHNAMVEGDFQAYVWWYIRRSYGPMKEDGTISKRGYAMAHFSKFVRPGYVRVEATKNPETNVYVSAYKGNKKLVIVAVNKNNSGVNQNFVLPNASVSKISRWITSGSSNLQPGTELTMTGGNFWAHLPAQSVTTFVADLG

SEQ ID NO: 12 - protein, 391 aa, Paenibacillus sp.
  mat_peptide (1)..(391)
ASDAIVNISAEKQVIRGFGGINHPVWIGDLTAAQRETAFGNGNNQLGFSILRIYVHEDRNQWYREVETAKRAIALGAIVFASPWNPPADMVETFNRNGDPSAKRLRYDKYAAYAQHLNDFVTYMRNNGVNLYAISVQNEPDYAHDWTWWTPQEMLRFMKENAGSINTRVIAPESFQYLKNMSDPILNDPQALANMDILGAHLYGTQVSNFAYPLFKQKGAGKDLWMTEVYYPNSDNNSADRWPEALDVSYHIHNAMVEGDFQAYVWWYIRRSYGPMKEDGTISKRGYAMAHFSKFVRPGYVRVEATKNPETNVYVSAYKGNKKLVIVAVNKNNSGVNQNFVLPNASVSKISRWITSGSSNLQPGTELTMTGGNFWAHLPAQSVTTFVADLG

SEQ ID NO: 13 - DNA, 1281 bp, Artificial Sequence
  Synthetic construct
  CDS (1)..(1278)
  sig_peptide (1)..(81)
  mat_peptide (82)..(1278)
atgaagaaaccgttggggaaaattgtcgcaagcaccgcactactcatttctgttgcttttagttcatcgatagcatcagcacatcatcatcaccatcatcctagggccagcgacgccatcgtaaatatttcggcggaaaaacaagtgattcgtggtttcggggggatcaaccatccggtctggatcggggatttgacggcggcacaaagagaaaccgccttcgggaacggaaataaccaactgggtttctcgattttgagaatctatgtacacgaagaccgaaatcaatggtaccgggaagtggaaacggccaaacgagcgattgccctcggagccatcgtattcgcatcgccatggaatccgcccgccgacatggtcgagaccttcaaccgaaacggcgatccgtcggcaaagcggctccgttatgacaagtatgccgcgtatgcccagcatcttaacgacttcgtgacgtatatgagaaacaacggcgtgaatctgtatgcgatttccgtccagaacgagcccgattatgcccatgactggacgtggtggacgccgcaggaaatgcttcgtttcatgaaagaaaatgccggatcgattaatacgagagtgatcgcgccggaatcgttccagtatctgaaaaatatgtcggacccgatactaaacgatccgcaggcgctagccaatatggatattcttggcgctcatctgtacgggacccaggtcagcaatttcgcttatcctttgttcaaacaaaaaggagcgggcaaagatctctggatgacggaggtatactatccgaacagtgacaacaactcggcggatcgttggcccgaagccctggatgtgtcctaccatatccacaatgcgatggtagagggagattttcaagcttacgtatggtggtatatccgcagatcgtacggtccgatgaaagaggacggcacgatcagcaaacgcggttacgctatggcgcatttctccaaattcgtccgtcccgggtatgtccgggttgaagcgacgaaaaatccggaaacgaacgtttatgtatccgcctataaaggtaataagaagcttgttatcgttgccgtcaacaaaaataactccggggtaaatcagaactttgtcctgcccaatgcatcggtttcgaaaatctccagatggatcactagcggcagcagcaatcttcagccgggaacggagcttacgatgacgggtggaaatttctgggctcatctcccggctcaaagcgtgaccacctttgtcgctgatcttggctaa

SEQ ID NO: 14 - protein, 426 aa, Artificial Sequence
  Synthetic Construct
MKKPLGKIVASTALLISVAFSSSIASAHHHHHHPRASDAIVNISAEKQVIRGFGGINHPVWIGDLTAAQRETAFGNGNNQLGFSILRIYVHEDRNQWYREVETAKRAIALGAIVFASPWNPPADMVETFNRNGDPSAKRLRYDKYAAYAQHLNDFVTYMRNNGVNLYAISVQNEPDYAHDWTWWTPQEMLRFMKENAGSINTRVIAPESFQYLKNMSDPILNDPQALANMDILGAHLYGTQVSNFAYPLFKQKGAGKDLWMTEVYYPNSDNNSADRWPEALDVSYHIHNAMVEGDFQAYVWWYIRRSYGPMKEDGTISKRGYAMAHFSKFVRPGYVRVEATKNPETNVYVSAYKGNKKLVIVAVNKNNSGVNQNFVLPNASVSKISRWITSGSSNLQPGTELTMTGGNFWAHLPAQSVTTFVADLG

SEQ ID NO: 15 - protein, 399 aa, Artificial Sequence
  Synthetic construct
  mat_peptide (1)..(399)
HHHHHHPRASDAIVNISAEKQVIRGFGGINHPVWIGDLTAAQRETAFGNGNNQLGFSILRIYVHEDRNQWYREVETAKRAIALGAIVFASPWNPPADMVETFNRNGDPSAKRLRYDKYAAYAQHLNDFVTYMRNNGVNLYAISVQNEPDYAHDWTWWTPQEMLRFMKENAGSINTRVIAPESFQYLKNMSDPILNDPQALANMDILGAHLYGTQVSNFAYPLFKQKGAGKDLWMTEVYYPNSDNNSADRWPEALDVSYHIHNAMVEGDFQAYVWWYIRRSYGPMKEDGTISKRGYAMAHFSKFVRPGYVRVEATKNPETNVYVSAYKGNKKLVIVAVNKNNSGVNQNFVLPNASVSKISRWITSGSSNLQPGTELTMTGGNFWAHLPAQSVTTFVADLG

SEQ ID NO: 16 - DNA, 1242 bp, Pectobacterium carotovorum
  CDS (1)..(1239)
  sig_peptide (1)..(90)
  mat_peptide (91)..(1239)
atgaatagaaatacagcttactgggtacgtaattgcttgcgtgcggcgatacttatttcggctgctacaagttcgcttgctatatatgctgatacggtaaagatcgatgcaaaaaccagttatcaaataattcaaggatttggcggcatgaatgcccctggatggattaacgatctgacaacagagcaagtcaataccgcattcggtaatgacacgggccaaatcgggctatcaattatgcgaatgagaattgatcctgatgccaacagatggaatatacaggtttcgagtgcacgtcaggcgtcattattaggcgcgaaattaatggcaacgccctggacaccgccagcttacatgaaaagtaacaaaagtctgatcaatggcgggcgtttactttcagaacactattctggctataccgagcatctgttaaaattttctaactttatgcaaactaacaacgcccctctttatgccatttcgatacaaaatgaacctgattggaaacctgactatgaatcctgtgaatggaatggtaatgattttaaaaactatctcaaatcacaaggctctaaattcggttctctgaaggtcattgtcggagagtcgctaaactttaaccatagtttaactgacccaacattgaatgatagtgaggcagcaaagcacgtggcgatcgtaggggggcacctctatggtacaacgcctaaaccgtatcctttagcccaaaacaagggtaaagaagtttggatgaccgaacacttagttgactcaaagcagtcagctaataactggtcatcagcgctcgaggttgcaagtgagatgaatgccagcatggttgcaaactataacgcttacgtgtggtggtatatccgccgctcttatgggttgcttactgaagatggcaaggtcagtaaacgtggttatgttatggcccaatatgccaaatttgtccgccctggcttccagcgaattcaagccacggaaaaccctcaggcaaatgttcatcttacagcctataaaaattcagaaggtaaaatggttattgttgctatcaatactaatgattcagaccaacttttatcgttgaatattagtaatcacacagtcagtaaatttgaaaaatatagcacatcggcaattttgaatgttgaatatggcggtacatataaggttgatagcaacggtaaatccagtgtatggctcaatccgttaagcgtaacaacctttgtcggaaaatga

SEQ ID NO: 17 - protein, 413 aa, Pectobacterium carotovorum
MNRNTAYWVRNCLRAAILISAATSSLAIYADTVKIDAKTSYQIIQGFGGMNAPGWINDLTTEQVNTAFGNDTGQIGLSIMRMRIDPDANRWNIQVSSARQASLLGAKLMATPWTPPAYMKSNKSLINGGRLLSEHYSGYTEHLLKFSNFMQTNNAPLYAISIQNEPDWKPDYESCEWNGNDFKNYLKSQGSKFGSLKVIVGESLNFNHSLTDPTLNDSEAAKHVAIVGGHLYGTTPKPYPLAQNKGKEVWMTEHLVDSKQSANNWSSALEVASEMNASMVANYNAYVWWYIRRSYGLLTEDGKVSKRGYVMAQYAKFVRPGFQRIQATENPQANVHLTAYKNSEGKMVIVAINTNDSDQLLSLNISNHTVSKFEKYSTSAILNVEYGGTYKVDSNGKSSVWLNPLSVTTFVGK

SEQ ID NO: 18 - protein, 383 aa, Pectobacterium carotovorum
  mat_peptide (1)..(383)
DTVKIDAKTSYQIIQGFGGMNAPGWINDLTTEQVNTAFGNDTGQIGLSIMRMRIDPDANRWNIQVSSARQASLLGAKLMATPWTPPAYMKSNKSLINGGRLLSEHYSGYTEHLLKFSNFMQTNNAPLYAISIQNEPDWKPDYESCEWNGNDFKNYLKSQGSKFGSLKVIVGESLNFNHSLTDPTLNDSEAAKHVAIVGGHLYGTTPKPYPLAQNKGKEVWMTEHLVDSKQSANNWSSALEVASEMNASMVANYNAYVWWYIRRSYGLLTEDGKVSKRGYVMAQYAKFVRPGFQRIQATENPQANVHLTAYKNSEGKMVIVAINTNDSDQLLSLNISNHTVSKFEKYSTSAILNVEYGGTYKVDSNGKSSVWLNPLSVTTFVGK

SEQ ID NO: 19 - DNA, 1257 bp, Artificial Sequence
  Synthetic construct
  CDS (1)..(1254)
  sig_peptide (1)..(81)
  mat_peptide (82)..(1254)
atgaagaaaccgttggggaaaattgtcgcaagcaccgcactactcatttctgttgcttttagttcatcgatagcatcagcacatcatcatcaccatcatcctagggatacagtcaaaattgatgcgaaaacaagctaccagattatccaaggctttggcggaatgaatgcaccgggatggattaatgatctgacaacagaacaagtcaatacggcgtttggcaatgatacaggccaaattggactgagcattatgcgcatgagaattgatccggatgcaaatagatggaatattcaagtttcaagcgcaagacaagcatcactgcttggcgcaaaactgatggcaacaccgtggacaccgcctgcatatatgaaatcaaataaaagcctgattaatggcggacgcctgctgtcagaacattattcaggctatacagaacatctgctgaaatttagcaactttatgcagacaaataacgcaccgctgtatgcgattagcattcaaaatgaaccggattggaaaccggattatgaatcatgcgaatggaatggcaacgactttaaaaactatctgaaaagccagggctcaaaatttggcagcctgaaagttattgttggcgaaagcctgaattttaaccattcactgacagatccgacactgaatgatagcgaagcagcaaaacatgttgcaattgttggaggccatctgtatggcacaacaccgaaaccgtatccgctggcacaaaataaaggcaaagaagtttggatgacggaacatctggttgattcaaaacaaagcgcgaataattggtcatcagcactggaagttgcatcagaaatgaatgcgtcaatggtcgcaaactataatgcatatgtctggtggtatattagacgctcatatggcctgcttacagaagatggcaaagtttcaaaacgcggatatgttatggcgcagtatgcaaaatttgttagaccgggatttcaacgcattcaggcaacagaaaatccgcaagcaaatgttcatctgacggcgtataaaaacagcgaaggcaaaatggttattgtcgcgattaatacgaacgattcagatcaactgctgagcctgaacattagcaatcatacagtcagcaaatttgaaaaatacagcacaagcgcgattctgaacgttgaatatggcggaacatataaagttgacagcaatggcaaatcaagcgtttggctgaatccgctgtcagttacaacatttgttggcaaataa

SEQ ID NO: 20 - protein, 418 aa, Artificial Sequence
  Synthetic Construct
MKKPLGKIVASTALLISVAFSSSIASAHHHHHHPRDTVKIDAKTSYQIIQGFGGMNAPGWINDLTTEQVNTAFGNDTGQIGLSIMRMRIDPDANRWNIQVSSARQASLLGAKLMATPWTPPAYMKSNKSLINGGRLLSEHYSGYTEHLLKFSNFMQTNNAPLYAISIQNEPDWKPDYESCEWNGNDFKNYLKSQGSKFGSLKVIVGESLNFNHSLTDPTLNDSEAAKHVAIVGGHLYGTTPKPYPLAQNKGKEVWMTEHLVDSKQSANNWSSALEVASEMNASMVANYNAYVWWYIRRSYGLLTEDGKVSKRGYVMAQYAKFVRPGFQRIQATENPQANVHLTAYKNSEGKMVIVAINTNDSDQLLSLNISNHTVSKFEKYSTSAILNVEYGGTYKVDSNGKSSVWLNPLSVTTFVGK

SEQ ID NO: 21 - protein, 391 aa, Artificial Sequence
  Synthetic construct
  mat_peptide (1)..(391)
HHHHHHPRDTVKIDAKTSYQIIQGFGGMNAPGWINDLTTEQVNTAFGNDTGQIGLSIMRMRIDPDANRWNIQVSSARQASLLGAKLMATPWTPPAYMKSNKSLINGGRLLSEHYSGYTEHLLKFSNFMQTNNAPLYAISIQNEPDWKPDYESCEWNGNDFKNYLKSQGSKFGSLKVIVGESLNFNHSLTDPTLNDSEAAKHVAIVGGHLYGTTPKPYPLAQNKGKEVWMTEHLVDSKQSANNWSSALEVASEMNASMVANYNAYVWWYIRRSYGLLTEDGKVSKRGYVMAQYAKFVRPGFQRIQATENPQANVHLTAYKNSEGKMVIVAINTNDSDQLLSLNISNHTVSKFEKYSTSAILNVEYGGTYKVDSNGKSSVWLNPLSVTTFVGK

SEQ ID NO: 22 - DNA, 1785 bp, Ruminococcus sp.
  CDS (1)..(1785)
  sig_peptide (1)..(90)
  mat_peptide (91)..(1785)
atgaattggagaaaagtcgcagctggttttacggcagcactgctgtctgtaacaacagcatccatttttccgacgaaaacggtttccgccgctgacgtttgtgtcattgatacagacacagaacatcagatgattcggggattcggcggaatcaaccatccggaatgggcaggagatctgacgcaggcacagcggcagacggcatttggaaacggagagaatgaactggggctgacggtacttcgggtgtttgtgaatccggacagcagccagtggagcagggcactccctactgcacagtttgcgacgcaaatgggtgtcactgtttttgcatcaccgtgggagccgccggcaaatctgacagaatccggcggaagtaacggcaagctgcatctgccaaaatccaattatgctgcctatgcaaaacatctgaatgattttggcacttacatgaaaaataataatgttgatttgtacgctatctctgtacagaatgaaccggattatgcgtccgagtggacatattggtctaccgatgaaacaacagacttcattgccaactatggcgatcagatcacaagcacccgcctgatgtcaccggaatcttttcagtatgcaccggaaaacgcctcatgggtgtcggacggcggcaagaaattttaccggaagatcttgaataacagcaaggcaatggcaaactgcgatgtgttcggtacgcacttttacggcacccagcgttcgtggatggattttccggatctggaaaacagcggaaaagaaatctggatgacagaagtctatgttcccaacagcgacaaggattcggcaaatcgttatccggaggcactgcaggtttcggaaaatattcataatgccatggttgtgggcaatatgagtgcatatacctggtggtatatccgccggaattatggtttgatgacggaagacggaaagatcagcaagcgtgggtactgcatggcacaatactccaaatatgttcgccccggcgatgtgcggatcgatgctacggaacagccggcagacaatgtctatgtgtctgcatataagggtgatgacaatcaggtaaccattgttgccattaacaagggaacagaaagctattcgcagcagttcgctgtagatgctgatgcacaaattacagaggttgaccgttatcgtacttccgcctctgaaaatctggcaaaaacagaaaatatggagcatgacagcagcagcttctgggcacagctccctgcggaaagtgtttctacgtttgttgtgacattggaagaccagccggtagaaccggatgagaacggctattattttcacgatacttttgagtcggacaactgcgactggcagggacacggctcagcagatattacactgagcggaagaataccgtatcagggaaccaatgcactcttggtacagaatcgtgccagtgcgtggaacggagcagaaaaggtactgccggcaaaagcatttcaggctggaaaagaatatagtttcagcgtatgcctgaattacatggacggagaaagttccaaaaatgctgcactttctctacagtacacagatgctgccggagaaacaaagtatgccagaattgcgtctgcttccgcagcaaagggaaactatgtgcagctcgcaaatcccagtttcaaacttccggatggcggaaagaattttaaaatttatgtggaaacggaaggcgatacagataatttctatattgatgaggcaatcggtgctgtgaaaggaactgcgattgaaggaccg

SEQ ID NO: 23 - protein, 595 aa, Ruminococcus sp.
MNWRKVAAGFTAALLSVTTASIFPTKTVSAADVCVIDTDTEHQMIRGFGGINHPEWAGDLTQAQRQTAFGNGENELGLTVLRVFVNPDSSQWSRALPTAQFATQMGVTVFASPWEPPANLTESGGSNGKLHLPKSNYAAYAKHLNDFGTYMKNNNVDLYAISVQNEPDYASEWTYWSTDETTDFIANYGDQITSTRLMSPESFQYAPENASWVSDGGKKFYRKILNNSKAMANCDVFGTHFYGTQRSWMDFPDLENSGKEIWMTEVYVPNSDKDSANRYPEALQVSENIHNAMVVGNMSAYTWWYIRRNYGLMTEDGKISKRGYCMAQYSKYVRPGDVRIDATEQPADNVYVSAYKGDDNQVTIVAINKGTESYSQQFAVDADAQITEVDRYRTSASENLAKTENMEHDSSSFWAQLPAESVSTFVVTLEDQPVEPDENGYYFHDTFESDNCDWQGHGSADITLSGRIPYQGTNALLVQNRASAWNGAEKVLPAKAFQAGKEYSFSVCLNYMDGESSKNAALSLQYTDAAGETKYARIASASAAKGNYVQLANPSFKLPDGGKNFKIYVETEGDTDNFYIDEAIGAVKGTAIEGP

SEQ ID NO: 24 - protein, 565 aa, Ruminococcus sp.
  mat_peptide (1)..(565)
ADVCVIDTDTEHQMIRGFGGINHPEWAGDLTQAQRQTAFGNGENELGLTVLRVFVNPDSSQWSRALPTAQFATQMGVTVFASPWEPPANLTESGGSNGKLHLPKSNYAAYAKHLNDFGTYMKNNNVDLYAISVQNEPDYASEWTYWSTDETTDFIANYGDQITSTRLMSPESFQYAPENASWVSDGGKKFYRKILNNSKAMANCDVFGTHFYGTQRSWMDFPDLENSGKEIWMTEVYVPNSDKDSANRYPEALQVSENIHNAMVVGNMSAYTWWYIRRNYGLMTEDGKISKRGYCMAQYSKYVRPGDVRIDATEQPADNVYVSAYKGDDNQVTIVAINKGTESYSQQFAVDADAQITEVDRYRTSASENLAKTENMEHDSSSFWAQLPAESVSTFVVTLEDQPVEPDENGYYFHDTFESDNCDWQGHGSADITLSGRIPYQGTNALLVQNRASAWNGAEKVLPAKAFQAGKEYSFSVCLNYMDGESSKNAALSLQYTDAAGETKYARIASASAAKGNYVQLANPSFKLPDGGKNFKIYVETEGDTDNFYIDEAIGAVKGTAIEGP

SEQ ID NO: 25 - DNA, 1803 bp, Artificial Sequence
  Synthetic construct
  CDS (1)..(1800)
  sig_peptide (1)..(81)
  mat_peptide (82)..(1800)
atgaagaaaccgttggggaaaattgtcgcaagcaccgcactactcatttctgttgcttttagttcatcgatagcatcagcacatcatcatcaccatcatcctagggcagatgtttgcgttattgatacagatacagaacaccagatgattagaggctttggcggaattaatcatccggaatgggcaggcgatctgacacaagcacaaagacaaacagcatttggcaatggcgaaaatgaactgggcctgacagttctgagagtttttgttaatccggatagctcacaatggtcaagagcactgccgacagcacaatttgcaacacaaatgggcgttacagtttttgcatcaccgtgggaaccgcctgcaaatctgacagaatcaggcggatcaaatggcaaactgcatctgccgaaatcaaattatgcagcgtatgcgaaacatctgaacgattttggcacgtatatgaaaaacaacaacgtcgatctgtatgcgattagcgttcaaaatgaaccggattatgcaagcgaatggacatattggagcacagatgaaacaacggattttattgcgaattatggcgatcagattacatcaacaagactgatgtcaccggaatcctttcaatatgcaccggaaaatgcatcatgggtttcagatggcggaaaaaaattttaccgcaaaatcctgaacaacagcaaagctatggcgaactgtgatgtttttggcacacatttttatggaacacagagaagctggatggattttcctgatcttgaaaatagcggcaaagaaatctggatgacggaagtttatgttccgaactcagataaagatagcgcgaatagatatccggaagcactgcaagttagcgaaaatattcataatgcaatggtcgtcggcaatatgtcagcatatacatggtggtatattcgcagaaattatggcctgatgacagaagatggcaaaatttcaaaacgcggatattgcatggcgcagtatagcaaatatgttagaccgggtgatgttcgcattgatgcaacagaacaaccggcagataatgtttatgttagcgcatataaaggcgacgataaccaagttacaattgtcgcaattaacaaaggcacggaaagctattcacaacaatttgcggttgatgcagatgcgcaaattacagaagttgatagatatagaacaagcgcgtcagaaaatctggcgaaaacagaaaatatggaacatgactcatcaagcttttgggcacaacttccggcagaatcagtttcaacatttgttgttacactggaagatcaaccggtcgaaccggatgaaaatggctattactttcatgacacgtttgaaagcgataactgcgattggcaaggccatggctcagcagatattacactttcaggcagaattccgtatcaaggcacaaatgcactgctggttcaaaatagagcatcagcatggaatggcgcagaaaaagttcttccggcaaaagcatttcaggctggcaaagaatattcatttagcgtgtgcctgaattacatggatggcgaatcatcaaaaaatgcagcactgtcactgcaatatacagatgcagcaggcgaaacaaaatatgcaagaattgcatcagcaagcgcagcgaaaggcaattatgttcaactggcaaatccgtcatttaaactgccggatggtggcaaaaactttaaaatctatgtcgaaacagaaggcgacacggacaacttttatatcgatgaagcaattggcgcagttaaaggcacagcaattgaaggaccgtaa

SEQ ID NO: 26 - protein, 600 aa, Artificial Sequence
  Synthetic Construct
MKKPLGKIVASTALLISVAFSSSIASAHHHHHHPRADVCVIDTDTEHQMIRGFGGINHPEWAGDLTQAQRQTAFGNGENELGLTVLRVFVNPDSSQWSRALPTAQFATQMGVTVFASPWEPPANLTESGGSNGKLHLPKSNYAAYAKHLNDFGTYMKNNNVDLYAISVQNEPDYASEWTYWSTDETTDFIANYGDQITSTRLMSPESFQYAPENASWVSDGGKKFYRKILNNSKAMANCDVFGTHFYGTQRSWMDFPDLENSGKEIWMTEVYVPNSDKDSANRYPEALQVSENIHNAMVVGNMSAYTWWYIRRNYGLMTEDGKISKRGYCMAQYSKYVRPGDVRIDATEQPADNVYVSAYKGDDNQVTIVAINKGTESYSQQFAVDADAQITEVDRYRTSASENLAKTENMEHDSSSFWAQLPAESVSTFVVTLEDQPVEPDENGYYFHDTFESDNCDWQGHGSADITLSGRIPYQGTNALLVQNRASAWNGAEKVLPAKAFQAGKEYSFSVCLNYMDGESSKNAALSLQYTDAAGETKYARIASASAAKGNYVQLANPSFKLPDGGKNFKIYVETEGDTDNFYIDEAIGAVKGTAIEGP

SEQ ID NO: 27 - protein, 573 aa, Artificial Sequence
  Synthetic construct
  mat_peptide (1)..(573)
HHHHHHPRADVCVIDTDTEHQMIRGFGGINHPEWAGDLTQAQRQTAFGNGENELGLTVLRVFVNPDSSQWSRALPTAQFATQMGVTVFASPWEPPANLTESGGSNGKLHLPKSNYAAYAKHLNDFGTYMKNNNVDLYAISVQNEPDYASEWTYWSTDETTDFIANYGDQITSTRLMSPESFQYAPENASWVSDGGKKFYRKILNNSKAMANCDVFGTHFYGTQRSWMDFPDLENSGKEIWMTEVYVPNSDKDSANRYPEALQVSENIHNAMVVGNMSAYTWWYIRRNYGLMTEDGKISKRGYCMAQYSKYVRPGDVRIDATEQPADNVYVSAYKGDDNQVTIVAINKGTESYSQQFAVDADAQITEVDRYRTSASENLAKTENMEHDSSSFWAQLPAESVSTFVVTLEDQPVEPDENGYYFHDTFESDNCDWQGHGSADITLSGRIPYQGTNALLVQNRASAWNGAEKVLPAKAFQAGKEYSFSVCLNYMDGESSKNAALSLQYTDAAGETKYARIASASAAKGNYVQLANPSFKLPDGGKNFKIYVETEGDTDNFYIDEAIGAVKGTAIEGP

SEQ ID NO: 28 - DNA, 1311 bp, Streptomyces sp.
  CDS (1)..(1308)
  sig_peptide (1)..(120)
  mat_peptide (121)..(1308)
atggcgtcacaccaccgcgcatccggcggcaccgggcccgaatcacccagccgccgcgccgttctggcgacgatgagtgccctgcccgtcctcgtggccgcgacaccggctgccggggcgtcccgcacaccgtccgcggcggctacctcggtgaccgtcgacccttcggccacccggcagacgatccggggattcggcggcatgaaccacccgctctggatcggcgacctgactcctgcccagcgggacacggccttcggcaacggcgagggccagctggggttctccgtgctgaggatccccgtatccgaggaccgggcgaactggagccgcgaagtcgcaacggccaagcgtgccaccgaactcggggcgatcgtcttcgcttcgccatggaatccgcccgccaacatggtcgagaccttcgtccgaggccagcagaccgacgcgaaacggctccgccactccatgtacggcgcctatgcccagcacctgaacgacttcgtcgcgttcatgaagagcaacggggtgaacctgtacgccatttccgtgcagaacgagcccgactacgcccatgactggacgtggtggaccccgagcgagatgaccaggttcctgcgggagaacgccgggtccatcagcaccaaggtcatcgccccggagtccttccagtacgtgaagacgttctccgacccgatcctcaacgacgccgcggcactcgccaacctggacattctcggggcgcacctctacggcacgtcgttccagaacttcccctatcccctcttcaagcagaagggcgggggcaaagaactctggatgaccgaggtctaccaccccaacagcagcgattcggccgatctgtggccccaggctctcgacgtcgcggaacacatccaccgcgcgatggtggacgccgagttccaggcgtacgtgtggtggtacatccggcgcggctacgggcccatgcgggaggacgggcggatcagcaaacgcggcgccggcatggcgcacttcagcaagttcgtccgccccgggcacgtacgggtcgccgtgacccccgctccccagccgaacgtctacctctcggcgtacaaagggggcggctcccgtgtggtcgtcgtcgccgtcaacaagggcgcctcgccggtgagccagcagttcaccctgaacaacaacaactcgtcgggcgtgtcgtcctgggtgaccgacgcgagcaggaacctcgcgtcccagggcagaatcaccgtggcgaacggcgccttcaccgcccggctcccggctcagagcgtgacgaccttggtcaccgggtag

SEQ ID NO: 29 - protein, 436 aa, Streptomyces sp.
MASHHRASGGTGPESPSRRAVLATMSALPVLVAATPAAGASRTPSAAATSVTVDPSATRQTIRGFGGMNHPLWIGDLTPAQRDTAFGNGEGQLGFSVLRIPVSEDRANWSREVATAKRATELGAIVFASPWNPPANMVETFVRGQQTDAKRLRHSMYGAYAQHLNDFVAFMKSNGVNLYAISVQNEPDYAHDWTWWTPSEMTRFLRENAGSISTKVIAPESFQYVKTFSDPILNDAAALANLDILGAHLYGTSFQNFPYPLFKQKGGGKELWMTEVYHPNSSDSADLWPQALDVAEHIHRAMVDAEFQAYVWWYIRRGYGPMREDGRISKRGAGMAHFSKFVRPGHVRVAVTPAPQPNVYLSAYKGGGSRVVVVAVNKGASPVSQQFTLNNNNSSGVSSWVTDASRNLASQGRITVANGAFTARLPAQSVTTLVTG

SEQ ID NO: 30 - protein, 396 aa, Streptomyces sp.
  mat_peptide (1)..(396)
SRTPSAAATSVTVDPSATRQTIRGFGGMNHPLWIGDLTPAQRDTAFGNGEGQLGFSVLRIPVSEDRANWSREVATAKRATELGAIVFASPWNPPANMVETFVRGQQTDAKRLRHSMYGAYAQHLNDFVAFMKSNGVNLYAISVQNEPDYAHDWTWWTPSEMTRFLRENAGSISTKVIAPESFQYVKTFSDPILNDAAALANLDILGAHLYGTSFQNFPYPLFKQKGGGKELWMTEVYHPNSSDSADLWPQALDVAEHIHRAMVDAEFQAYVWWYIRRGYGPMREDGRISKRGAGMAHFSKFVRPGHVRVAVTPAPQPNVYLSAYKGGGSRVVVVAVNKGASPVSQQFTLNNNNSSGVSSWVTDASRNLASQGRITVANGAFTARLPAQSVTTLVTG

SEQ ID NO: 31 - DNA, 1296 bp, Artificial Sequence
  Synthetic construct
  CDS (1)..(1293)
  sig_peptide (1)..(81)
  mat_peptide (82)..(1293)
atgaagaaaccgttggggaaaattgtcgcaagcaccgcactactcatttctgttgcttttagttcatcgatagcatcagcacatcatcatcaccatcatcctaggtcccgcacaccgtccgcggcggctacctcggtgaccgtcgacccttcggccacccggcagacgatccggggattcggcggcatgaaccacccgctctggatcggcgacctgactcctgcccagcgggacacggccttcggcaacggcgagggccagctggggttctccgtgctgaggatccccgtatccgaggaccgggcgaactggagccgcgaagtcgcaacggccaagcgtgccaccgaactcggggcgatcgtcttcgcttcgccatggaatccgcccgccaacatggtcgagaccttcgtccgaggccagcagaccgacgcgaaacggctccgccactccatgtacggcgcctatgcccagcacctgaacgacttcgtcgcgttcatgaagagcaacggggtgaacctgtacgccatttccgtgcagaacgagcccgactacgcccatgactggacgtggtggaccccgagcgagatgaccaggttcctgcgggagaacgccgggtccatcagcaccaaggtcatcgccccggagtccttccagtacgtgaagacgttctccgacccgatcctcaacgacgccgcggcactcgccaacctggacattctcggggcgcacctctacggcacgtcgttccagaacttcccctatcccctcttcaagcagaagggcgggggcaaagaactctggatgaccgaggtctaccaccccaacagcagcgattcggccgatctgtggccccaggctctcgacgtcgcggaacacatccaccgcgcgatggtggacgccgagttccaggcgtacgtgtggtggtacatccggcgcggctacgggcccatgcgggaggacgggcggatcagcaaacgcggcgccggcatggcgcacttcagcaagttcgtccgccccgggcacgtacgggtcgccgtgacccccgctccccagccgaacgtctacctctcggcgtacaaagggggcggctcccgtgtggtcgtcgtcgccgtcaacaagggcgcctcgccggtgagccagcagttcaccctgaacaacaacaactcgtcgggcgtgtcgtcctgggtgaccgacgcgagcaggaacctcgcgtcccagggcagaatcaccgtggcgaacggcgccttcaccgcccggctcccggctcagagcgtgacgaccttggtcaccgggtaa

SEQ ID NO: 32 - protein, 431 aa, Artificial Sequence
  Synthetic Construct
MKKPLGKIVASTALLISVAFSSSIASAHHHHHHPRSRTPSAAATSVTVDPSATRQTIRGFGGMNHPLWIGDLTPAQRDTAFGNGEGQLGFSVLRIPVSEDRANWSREVATAKRATELGAIVFASPWNPPANMVETFVRGQQTDAKRLRHSMYGAYAQHLNDFVAFMKSNGVNLYAISVQNEPDYAHDWTWWTPSEMTRFLRENAGSISTKVIAPESFQYVKTFSDPILNDAAALANLDILGAHLYGTSFQNFPYPLFKQKGGGKELWMTEVYHPNSSDSADLWPQALDVAEHIHRAMVDAEFQAYVWWYIRRGYGPMREDGRISKRGAGMAHFSKFVRPGHVRVAVTPAPQPNVYLSAYKGGGSRVVVVAVNKGASPVSQQFTLNNNNSSGVSSWVTDASRNLASQGRITVANGAFTARLPAQSVTTLVTG

SEQ ID NO: 33 - protein, 404 aa, Artificial Sequence
  Synthetic construct
  mat_peptide (1)..(404)
HHHHHHPRSRTPSAAATSVTVDPSATRQTIRGFGGMNHPLWIGDLTPAQRDTAFGNGEGQLGFSVLRIPVSEDRANWSREVATAKRATELGAIVFASPWNPPANMVETFVRGQQTDAKRLRHSMYGAYAQHLNDFVAFMKSNGVNLYAISVQNEPDYAHDWTWWTPSEMTRFLRENAGSISTKVIAPESFQYVKTFSDPILNDAAALANLDILGAHLYGTSFQNFPYPLFKQKGGGKELWMTEVYHPNSSDSADLWPQALDVAEHIHRAMVDAEFQAYVWWYIRRGYGPMREDGRISKRGAGMAHFSKFVRPGHVRVAVTPAPQPNVYLSAYKGGGSRVVVVAVNKGASPVSQQFTLNNNNSSGVSSWVTDASRNLASQGRITVANGAFTARLPAQSVTTLVTG

SEQ ID NO: 34 - DNA, 1272 bp, Clostridium saccharobutylicum
  CDS (1)..(1269)
  sig_peptide (1)..(93)
  mat_peptide (94)..(1269)
atgaatattaagataaaaaagatattgctatctttattaacagtttctatgacaagttcactatttgtaggattatcaacaacagctaatgctgcatcgaatgctacaataaatttatcagcacaaaaacaagttattagaggtttcggaggaataaatcttcctgcttgggctggagatttgacagcagctcaaagagaaacagcttttggcaatggagataatcagttaggtctttcggttttaagaatctatgtagatgataacaaaaataattggtacaaagaattagcaactgcaaaaaaagcgattgaacacggagcaatagtttttgctacaccatggaatccaccagcttatatgactgagaaattcaatcgtaatggtgatacaaatgcaaaacgacttagatacgataagtatgctgcatatgctcaacatcttaatgattttgtttcatacatgaaaaataatggtgttaatctttatgctatttcagttcaaaatgaacctgattatggaaaagagtggacatggtggactccacaagaaatacttagatttatcaaagaaaatgctggatcaattaattgtagggtaatgtcacctgagtcattttcttatcaaaaaaacatgtacgaccctattttgaataatccacaggcacttgctaatatggatattcttggtactcatacttatggtactagagttaacgattttgcttatcctctttttaaacaaaaaggagcaggaaaagaactttggatgacagaagtgtatgttccaaatagtgatactaactcagcagatagatggccagaagcattagatgttgcagatcatataaacaatgcaatggtagagggagattttcaggcatatgtatggtggtacatccgtagatcatatggtttcattaaagaagacggtaatgtaagtaagcgtggttacatgatggctcatttttctaagtttgttcgtcctggatatgtaagggttgatgcaacaaagaatccaacaccaaatgtttacttatctgcgtataaaggtaataacaaagtagttattattgcaataaacaaaggaacatctgatgtaaaacaaagcttcactatgccaaatagcaaagtttctagtgtatcatcatggcaaactacagcaacagcaaatttggctaaatcagcttcaaacacaaatgtatacaatggaaactttacagcaacacttccagcacaaagtgtaactacatttgtaggagatattaagtaa

SEQ ID NO: 35 - protein, 423 aa, Clostridium saccharobutylicum
MNIKIKKILLSLLTVSMTSSLFVGLSTTANAASNATINLSAQKQVIRGFGGINLPAWAGDLTAAQRETAFGNGDNQLGLSVLRIYVDDNKNNWYKELATAKKAIEHGAIVFATPWNPPAYMTEKFNRNGDTNAKRLRYDKYAAYAQHLNDFVSYMKNNGVNLYAISVQNEPDYGKEWTWWTPQEILRFIKENAGSINCRVMSPESFSYQKNMYDPILNNPQALANMDILGTHTYGTRVNDFAYPLFKQKGAGKELWMTEVYVPNSDTNSADRWPEALDVADHINNAMVEGDFQAYVWWYIRRSYGFIKEDGNVSKRGYMMAHFSKFVRPGYVRVDATKNPTPNVYLSAYKGNNKVVIIAINKGTSDVKQSFTMPNSKVSSVSSWQTTATANLAKSASNTNVYNGNFTATLPAQSVTTFVGDIK

SEQ ID NO: 36 - protein, 392 aa, Clostridium saccharobutylicum
  mat_peptide (1)..(392)
ASNATINLSAQKQVIRGFGGINLPAWAGDLTAAQRETAFGNGDNQLGLSVLRIYVDDNKNNWYKELATAKKAIEHGAIVFATPWNPPAYMTEKFNRNGDTNAKRLRYDKYAAYAQHLNDFVSYMKNNGVNLYAISVQNEPDYGKEWTWWTPQEILRFIKENAGSINCRVMSPESFSYQKNMYDPILNNPQALANMDILGTHTYGTRVNDFAYPLFKQKGAGKELWMTEVYVPNSDTNSADRWPEALDVADHINNAMVEGDFQAYVWWYIRRSYGFIKEDGNVSKRGYMMAHFSKFVRPGYVRVDATKNPTPNVYLSAYKGNNKVVIIAINKGTSDVKQSFTMPNSKVSSVSSWQTTATANLAKSASNTNVYNGNFTATLPAQSVTTFVGDIK

SEQ ID NO: 37 - DNA, 1284 bp, Artificial Sequence
  Synthetic construct
  CDS (1)..(1281)
  sig_peptide (1)..(81)
  mat_peptide (82)..(1281)
atgaagaaaccgttggggaaaattgtcgcaagcaccgcactactcatttctgttgcttttagttcatcgatagcatcagcacatcatcatcaccatcatcctagggcatcaaatgcaacaattaatctgagcgcacagaaacaagtcattagaggctttggcggaattaatcttccggcatgggcaggcgatctgacagcagcacaaagagaaacagcatttggcaatggcgataatcaactgggcctgtcagttctgagaatttatgtcgatgacaacaaaaacaactggtataaagaactggcgacagcgaaaaaagcaattgaacatggcgcaattgtttttgcaacaccgtggaatccgcctgcatatatgacagaaaaatttaatcgcaacggcgacacaaatgcgaaaagactgagatatgataaatatgcagcgtatgcgcagcatctgaatgattttgttagctatatgaaaaacaacggcgtcaacctgtatgcaattagcgttcaaaatgaaccggactatggcaaagaatggacatggtggacaccgcaagaaattctgcgctttatcaaagaaaatgcaggcagcattaattgcagagttatgtcaccggaaagctttagctaccagaaaaatatgtatgacccgattctgaataatccgcaagcactggcaaatatggatattctgggcacacatacatatggcacacgcgttaacgattttgcatatccgctttttaaacaaaaaggcgcaggcaaagaactgtggatgacggaagtttatgttccgaattcagatacaaattcagcggatagatggcctgaagcactggatgttgcagatcatattaacaatgcaatggtcgaaggcgattttcaagcatatgtttggtggtatattagacgcagctatggatttatcaaagaagatggcaacgttagcaaacgcggatatatgatggcacattttagcaaatttgtccgtccgggatatgttagagttgatgcgacaaaaaatccgacaccgaatgtttatctgtcagcgtataaaggcaataacaaagtcgtcattatcgcgatcaataaaggcacgtcagatgtcaaacaaagctttacaatgccgaatagcaaagtttcatcagtttcaagctggcaaacaacagcaacagcaaatctggcaaaatcagcgagcaatacaaatgtctataacggcaattttacagcgacacttccggcacaatcagttacaacatttgttggcgatatcaaataa

SEQ ID NO: 38 - protein, 427 aa, Artificial Sequence
  Synthetic Construct
MKKPLGKIVASTALLISVAFSSSIASAHHHHHHPRASNATINLSAQKQVIRGFGGINLPAWAGDLTAAQRETAFGNGDNQLGLSVLRIYVDDNKNNWYKELATAKKAIEHGAIVFATPWNPPAYMTEKFNRNGDTNAKRLRYDKYAAYAQHLNDFVSYMKNNGVNLYAISVQNEPDYGKEWTWWTPQEILRFIKENAGSINCRVMSPESFSYQKNMYDPILNNPQALANMDILGTHTYGTRVNDFAYPLFKQKGAGKELWMTEVYVPNSDTNSADRWPEALDVADHINNAMVEGDFQAYVWWYIRRSYGFIKEDGNVSKRGYMMAHFSKFVRPGYVRVDATKNPTPNVYLSAYKGNNKVVIIAINKGTSDVKQSFTMPNSKVSSVSSWQTTATANLAKSASNTNVYNGNFTATLPAQSVTTFVGDIK

SEQ ID NO: 39 - protein, 400 aa, Artificial Sequence
  Synthetic construct
  mat_peptide (1)..(400)
HHHHHHPRASNATINLSAQKQVIRGFGGINLPAWAGDLTAAQRETAFGNGDNQLGLSVLRIYVDDNKNNWYKELATAKKAIEHGAIVFATPWNPPAYMTEKFNRNGDTNAKRLRYDKYAAYAQHLNDFVSYMKNNGVNLYAISVQNEPDYGKEWTWWTPQEILRFIKENAGSINCRVMSPESFSYQKNMYDPILNNPQALANMDILGTHTYGTRVNDFAYPLFKQKGAGKELWMTEVYVPNSDTNSADRWPEALDVADHINNAMVEGDFQAYVWWYIRRSYGFIKEDGNVSKRGYMMAHFSKFVRPGYVRVDATKNPTPNVYLSAYKGNNKVVIIAINKGTSDVKQSFTMPNSKVSSVSSWQTTATANLAKSASNTNVYNGNFTATLPAQSVTTFVGDIK

SEQ ID NO: 40 - DNA, 1341 bp, Paenibacillus panacisoli
  CDS (1)..(1338)
  sig_peptide (1)..(99)
  mat_peptide (100)..(1338)
atgatttcacgtgtcaaaaagaccatttgttcactattggcctgcgtgaccgcattgccgctgcttttgagcctgcctgccgctgacccggtatctgcggcaagtgatgctgtcattaatttatcagcacaaaagcaagttattcgtgggtttggaggaatcaaccatcctgcatggattggggatctgacagccgcccaacgtgagactgcattcggtaacggacaaaatcagttaggcttctccatactgagagtgtatatagaccccgatcgtaataactggagtcgagaagtagccacggctaaaaaggctattgaaaaaggagcacttgtcttcgcttccccatggaatccgccaagcagtatggtcgagaccttcaaccggaacggcgacagaaatgccaagcggctcagatatgacaagtacgccgcttatgcacagcatcttaatgatttcgtgacttatatgaaaaataatggtgtgaacctctacgctatctctgtacaaaatgaacctgactatgcccatgagtggacatggtggactccacaggaaatccttcgcttcatgaaggaaaacgctggctccatcaattgcaaggtgatggcgcccgaatcgttccaatatctcaagaatatatccgacccgatcctgaatgatccgcaggcgcttgccaacatggacatcctcggtgctcatctgtatggaacccaggtcagcaacttcgcctatccgctcttcaagcaaaaaggagcaggcaaagagctatggatgacagaagtatactaccctaatagtgacaacaactcggcggaccgctggcctgaagccctggaggtctcccatcatatgcataatgctatggtagaaggcgacttccaagcttatgtatggtggtatatccgcagaagttacggccctatgaaggaagacggcaccatcagcaagcgtggctacaatatggctcatttctctaaatttatccgtcctggctatgtgcgcgtagatgcgaccaagaatccggatacgaacgtgcatgtctctgcttacaaaggcaacaacaaggtcgttatcgtagccattaaccgtggaacaacagctgtcaaccaaaatttcgtcctgcaaaatggtagagcagccacgttatcgagatggatcacagatgcgaatcggaatctggcacccgaatccaacctgaatgcatccagcggctcattctttgcacacctgccagccaaaagtgtcaccaccttcgtgggtgatttgacaggctcaaccctgaatattgaggatgctgctctgacaaacagcgtgacacaagacacctattccaaataa

SEQ ID NO: 41 - protein, 446 aa, Paenibacillus panacisoli
MISRVKKTICSLLACVTALPLLLSLPAADPVSAASDAVINLSAQKQVIRGFGGINHPAWIGDLTAAQRETAFGNGQNQLGFSILRVYIDPDRNNWSREVATAKKAIEKGALVFASPWNPPSSMVETFNRNGDRNAKRLRYDKYAAYAQHLNDFVTYMKNNGVNLYAISVQNEPDYAHEWTWWTPQEILRFMKENAGSINCKVMAPESFQYLKNISDPILNDPQALANMDILGAHLYGTQVSNFAYPLFKQKGAGKELWMTEVYYPNSDNNSADRWPEALEVSHHMHNAMVEGDFQAYVWWYIRRSYGPMKEDGTISKRGYNMAHFSKFIRPGYVRVDATKNPDTNVHVSAYKGNNKVVIVAINRGTTAVNQNFVLQNGRAATLSRWITDANRNLAPESNLNASSGSFFAHLPAKSVTTFVGDLTGSTLNIEDAALTNSVTQDTYSK

SEQ ID NO: 42 - protein, 413 aa, Paenibacillus panacisoli
  mat_peptide (1)..(413)
ASDAVINLSAQKQVIRGFGGINHPAWIGDLTAAQRETAFGNGQNQLGFSILRVYIDPDRNNWSREVATAKKAIEKGALVFASPWNPPSSMVETFNRNGDRNAKRLRYDKYAAYAQHLNDFVTYMKNNGVNLYAISVQNEPDYAHEWTWWTPQEILRFMKENAGSINCKVMAPESFQYLKNISDPILNDPQALANMDILGAHLYGTQVSNFAYPLFKQKGAGKELWMTEVYYPNSDNNSADRWPEALEVSHHMHNAMVEGDFQAYVWWYIRRSYGPMKEDGTISKRGYNMAHFSKFIRPGYVRVDATKNPDTNVHVSAYKGNNKVVIVAINRGTTAVNQNFVLQNGRAATLSRWITDANRNLAPESNLNASSGSFFAHLPAKSVTTFVGDLTGSTLNIEDAALTNSVTQDTYSK

SEQ ID NO: 43 - DNA, 1347 bp, Artificial Sequence
  Synthetic construct
  CDS (1)..(1344)
  sig_peptide (1)..(81)
  mat_peptide (82)..(1344)
atgaagaaaccgttggggaaaattgtcgcaagcaccgcactactcatttctgttgcttttagttcatcgatagcatcagcacatcatcatcaccatcatcctagggcatcagatgcagttattaatctgtcagcacagaaacaagtcattagaggctttggcggaattaatcatccggcatggattggcgatctgacagcagcacaaagagaaacagcatttggcaatggccaaaatcaactgggcttttcaattctgcgcgtttatattgatccggacagaaataattggagcagagaagttgcaacagcgaaaaaagcaattgaaaaaggcgcactggtttttgcatcaccgtggaatccgccttcatcaatggttgaaacatttaatagaaacggcgatcgcaatgcgaaaagactgcgctatgataaatatgcagcatatgcgcaacatctgaacgactttgtcacgtacatgaaaaataacggcgttaacctgtatgcgattagcgttcaaaatgaaccggattatgcacatgaatggacatggtggacaccgcaagaaattctgagatttatgaaagaaaacgcaggcagcattaactgcaaagttatggcaccggaatcatttcagtacctgaaaaacatttcagacccgattctgaatgatccgcaagcactggcaaatatggatattcttggcgcacatctgtatggcacacaagtttcaaattttgcgtacccgctttttaaacagaaaggcgcaggcaaagaactgtggatgacagaagtttattatccgaacagcgataacaatagcgcagatagatggcctgaagcactggaagtttcacatcatatgcataatgcaatggtcgaaggcgattttcaagcatatgtttggtggtatattcgcagatcatatggaccgatgaaagaagatggcacaatttcaaaacgcggatataacatggcgcattttagcaagtttattcgtccgggatatgttagagttgatgcgacaaaaaatccggatacaaatgttcatgtcagcgcgtataaaggcaataacaaagttgtcattgtcgcgattaatagaggcacaacagcagtcaatcagaattttgttctgcaaaatggcagagcagcaacactgtcaagatggattacagatgcaaatagaaatcttgcaccggaaagcaatctgaatgcatcatcaggctcattttttgcacatcttccggcaaaaagcgtcacaacatttgttggagatcttacaggctcaacactgaatattgaagatgcagcactgacaaattcagtcacacaagatacgtacagcaaataa

SEQ ID NO: 44 - protein, 448 aa, Artificial Sequence
  Synthetic Construct
MKKPLGKIVASTALLISVAFSSSIASAHHHHHHPRASDAVINLSAQKQVIRGFGGINHPAWIGDLTAAQRETAFGNGQNQLGFSILRVYIDPDRNNWSREVATAKKAIEKGALVFASPWNPPSSMVETFNRNGDRNAKRLRYDKYAAYAQHLNDFVTYMKNNGVNLYAISVQNEPDYAHEWTWWTPQEILRFMKENAGSINCKVMAPESFQYLKNISDPILNDPQALANMDILGAHLYGTQVSNFAYPLFKQKGAGKELWMTEVYYPNSDNNSADRWPEALEVSHHMHNAMVEGDFQAYVWWYIRRSYGPMKEDGTISKRGYNMAHFSKFIRPGYVRVDATKNPDTNVHVSAYKGNNKVVIVAINRGTTAVNQNFVLQNGRAATLSRWITDANRNLAPESNLNASSGSFFAHLPAKSVTTFVGDLTGSTLNIEDAALTNSVTQDTYSK

SEQ ID NO: 45 - protein, 421 aa, Artificial Sequence
  Synthetic construct
  mat_peptide (1)..(421)
HHHHHHPRASDAVINLSAQKQVIRGFGGINHPAWIGDLTAAQRETAFGNGQNQLGFSILRVYIDPDRNNWSREVATAKKAIEKGALVFASPWNPPSSMVETFNRNGDRNAKRLRYDKYAAYAQHLNDFVTYMKNNGVNLYAISVQNEPDYAHEWTWWTPQEILRFMKENAGSINCKVMAPESFQYLKNISDPILNDPQALANMDILGAHLYGTQVSNFAYPLFKQKGAGKELWMTEVYYPNSDNNSADRWPEALEVSHHMHNAMVEGDFQAYVWWYIRRSYGPMKEDGTISKRGYNMAHFSKFIRPGYVRVDATKNPDTNVHVSAYKGNNKVVIVAINRGTTAVNQNFVLQNGRAATLSRWITDANRNLAPESNLNASSGSFFAHLPAKSVTTFVGDLTGSTLNIEDAALTNSVTQDTYSK

SEQ ID NO: 46 - DNA, 1194 bp, Unknown
  Human Stool metagenome
  CDS (1)..(1194)
  mat_peptide (1)..(1194)
gcacaagcagcatcagatgcagttattaatctgaacaacacgcatcaagaaatcatgggctttggcggaatgaatcatccgacatgggcaggcgatctgacatcatcacaaagagaaacagcatttggcaatggcacaaatcaactgggctttcaagttctgagaatttgggttgatagcgatcgcaacaactggtataaagaactggcaacagcaaaagcagcactggcaaaaggcgcaattgtttttgcaacaccgtggaatccgccttcaaatctgtgcgaaacattttacaaaaatggcagcgcaaatgcgaaacgcctgaaacatgataaatatgcagcatatgcgcagcacctgaatgattttgttacatatatgcgcaataacggcgttgaactgtatggcatttcagtttgcaatgaaccggattatggccatgattggacatggtggacagaatcagaagttgtcacatttctgaaatattacgcaggcagcattaactgcagaattattgcaccggaaagctttagctatcagaaaagctattatgatgcgatcatcaatgattcacaagcacttgcacaagtcgatatcattggcacacatctttatggcacaagctacaacaattttagctacccgctgtatcatcaaaaagcgtcatcaaaacaactgtggatgacagaagtttatacaccgaattcaacaagcagcgcagataaatggcctgaagcaattaatgttgcggaacacattcataaagccatggtcaatgattttcagacgtatgtctggtggtatattagaagatcatacggaccgatgaaagaagatggcacactgtcaaaacgcggatattgcatggcacagtttagcaaatttatccgcagaggctataaacgcgttgatgcaacagaaaatccgaacaatggcgtttatgttagcgcatatacaggcgacggcaaagcagttattgttgcagtcaattcaggctcatcagattgctcacaaagctttacaatcaaaggcaaaacgctgaaaaacgtcgatagatatagaacatcaggcagcgaaaatctggccaaaacatcaaatctggaactgtcaggcaatggcttttgggcatatcttccggcaaattcagttagcacatttgtttgcacagtcgaaaattcatcaagcaacccg

SEQ ID NO: 47 - protein, 398 aa, Unknown
  Synthetic Construct
AQAASDAVINLNNTHQEIMGFGGMNHPTWAGDLTSSQRETAFGNGTNQLGFQVLRIWVDSDRNNWYKELATAKAALAKGAIVFATPWNPPSNLCETFYKNGSANAKRLKHDKYAAYAQHLNDFVTYMRNNGVELYGISVCNEPDYGHDWTWWTESEVVTFLKYYAGSINCRIIAPESFSYQKSYYDAIINDSQALAQVDIIGTHLYGTSYNNFSYPLYHQKASSKQLWMTEVYTPNSTSSADKWPEAINVAEHIHKAMVNDFQTYVWWYIRRSYGPMKEDGTLSKRGYCMAQFSKFIRRGYKRVDATENPNNGVYVSAYTGDGKAVIVAVNSGSSDCSQSFTIKGKTLKNVDRYRTSGSENLAKTSNLELSGNGFWAYLPANSVSTFVCTVENSSSNP

SEQ ID NO: 48 - protein, 398 aa, Unknown; identical to SEQ ID NO: 47
  Human Stool metagenome
  mat_peptide (1)..(398)

SEQ ID NO: 49 - DNA, 1302 bp, Artificial Sequence
  Synthetic construct
  CDS (1)..(1299)
  sig_peptide (1)..(81)
  mat_peptide (82)..(1299)
atgaagaaaccgttggggaaaattgtcgcaagcaccgcactactcatttctgttgcttttagttcatcgatagcatcagcacatcatcatcaccatcatcctagggcacaagcagcatcagatgcagttattaatctgaacaacacgcatcaagaaatcatgggctttggcggaatgaatcatccgacatgggcaggcgatctgacatcatcacaaagagaaacagcatttggcaatggcacaaatcaactgggctttcaagttctgagaatttgggttgatagcgatcgcaacaactggtataaagaactggcaacagcaaaagcagcactggcaaaaggcgcaattgtttttgcaacaccgtggaatccgccttcaaatctgtgcgaaacattttacaaaaatggcagcgcaaatgcgaaacgcctgaaacatgataaatatgcagcatatgcgcagcacctgaatgattttgttacatatatgcgcaataacggcgttgaactgtatggcatttcagtttgcaatgaaccggattatggccatgattggacatggtggacagaatcagaagttgtcacatttctgaaatattacgcaggcagcattaactgcagaattattgcaccggaaagctttagctatcagaaaagctattatgatgcgatcatcaatgattcacaagcacttgcacaagtcgatatcattggcacacatctttatggcacaagctacaacaattttagctacccgctgtatcatcaaaaagcgtcatcaaaacaactgtggatgacagaagtttatacaccgaattcaacaagcagcgcagataaatggcctgaagcaattaatgttgcggaacacattcataaagccatggtcaatgattttcagacgtatgtctggtggtatattagaagatcatacggaccgatgaaagaagatggcacactgtcaaaacgcggatattgcatggcacagtttagcaaatttatccgcagaggctataaacgcgttgatgcaacagaaaatccgaacaatggcgtttatgttagcgcatatacaggcgacggcaaagcagttattgttgcagtcaattcaggctcatcagattgctcacaaagctttacaatcaaaggcaaaacgctgaaaaacgtcgatagatatagaacatcaggcagcgaaaatctggccaaaacatcaaatctggaactgtcaggcaatggcttttgggcatatcttccggcaaattcagttagcacatttgtttgcacagtcgaaaattcatcaagcaacccgtaa

SEQ ID NO: 50 - protein, 433 aa, Artificial Sequence
  Synthetic Construct
MKKPLGKIVASTALLISVAFSSSIASAHHHHHHPRAQAASDAVINLNNTHQEIMGFGGMNHPTWAGDLTSSQRETAFGNGTNQLGFQVLRIWVDSDRNNWYKELATAKAALAKGAIVFATPWNPPSNLCETFYKNGSANAKRLKHDKYAAYAQHLNDFVTYMRNNGVELYGISVCNEPDYGHDWTWWTESEVVTFLKYYAGSINCRIIAPESFSYQKSYYDAIINDSQALAQVDIIGTHLYGTSYNNFSYPLYHQKASSKQLWMTEVYTPNSTSSADKWPEAINVAEHIHKAMVNDFQTYVWWYIRRSYGPMKEDGTLSKRGYCMAQFSKFIRRGYKRVDATENPNNGVYVSAYTGDGKAVIVAVNSGSSDCSQSFTIKGKTLKNVDRYRTSGSENLAKTSNLELSGNGFWAYLPANSVSTFVCTVENSSSNP

SEQ ID NO: 51 - protein, 406 aa, Artificial Sequence
  Synthetic construct
  mat_peptide (1)..(406)
HHHHHHPRAQAASDAVINLNNTHQEIMGFGGMNHPTWAGDLTSSQRETAFGNGTNQLGFQVLRIWVDSDRNNWYKELATAKAALAKGAIVFATPWNPPSNLCETFYKNGSANAKRLKHDKYAAYAQHLNDFVTYMRNNGVELYGISVCNEPDYGHDWTWWTESEVVTFLKYYAGSINCRIIAPESFSYQKSYYDAIINDSQALAQVDIIGTHLYGTSYNNFSYPLYHQKASSKQLWMTEVYTPNSTSSADKWPEAINVAEHIHKAMVNDFQTYVWWYIRRSYGPMKEDGTLSKRGYCMAQFSKFIRRGYKRVDATENPNNGVYVSAYTGDGKAVIVAVNSGSSDCSQSFTIKGKTLKNVDRYRTSGSENLAKTSNLELSGNGFWAYLPANSVSTFVCTVENSSSNP

SEQ ID NO: 52 - DNA, 1149 bp, Vibrio rhizosphaerae
  CDS (1)..(1146)
  mat_peptide (1)..(1146)
ggctcagtctatattaactttaacacggaatatcaagaaatcgacggctttggcgcaatgaatgcaccgggatgggttaatgatctgacatcagcacaagcaacaaaagcatttggcaatggcgacggccaaatgggcctgtcaattatgagaatgagaattgatccggatagcaatcagtggtatagacaagttccgacagcacaaattgcatattcatatggcgcaaaactgctggcaacaccgtggtcaccgcctgcatatatgaaaacaaataacaacgtcaacaacggtggcaaactgaaaaaagaacattattggggctatacggatcatctgatggactttacaaactacatggcaggcaaaaacgcaccgatttatgcactgtcaattcagaatgaaccggattggcatccgaattatgaatcatgcgattggtcaggcgcagactttgttaattatctgaatgatcaaggctggcgtctggattcatcactgaaaattctggcaccggaatcactgggctttaatccggcactgtcagatccgattctgaaagattcagttgcaagcagccatattgatattattggcggacatctgtatggcgttcaaccgagaaattatccgctggcactgcaaaaaggcaaaaaactgtggatgacggaacattatacagatacagataacgcgaacatctgggacaaagctatgaatgttggcctggaactgcatcaatcaatggtttcaaattatagcgcgtatatctggtggtatcttagaagatcatatggcatgctgacagaagatggcaatatttcaaaacgcggatatatcatgagccagtttagcaagtttattagaccgggtgatgttagaattgcagcaacggaagttccggaaagcaatgtttatgttacggcgtataaaaacagaagcggcaaactggttattgcggtcgtcaataaaacaaacagccataaaaaactggattttacgctgcaaaatggcacagttggcagcatgacaaaatatgttacaagcgcatcacagaatgtcggctatgcaggcaaatatgcagttagcaataatcgctttacagcatatgcagatccgctgtcagttcaaacatttgtttcagaataa

SEQ ID NO: 53 - protein, 382 aa, Vibrio rhizosphaerae
GSVYINFNTEYQEIDGFGAMNAPGWVNDLTSAQATKAFGNGDGQMGLSIMRMRIDPDSNQWYRQVPTAQIAYSYGAKLLATPWSPPAYMKTNNNVNNGGKLKKEHYWGYTDHLMDFTNYMAGKNAPIYALSIQNEPDWHPNYESCDWSGADFVNYLNDQGWRLDSSLKILAPESLGFNPALSDPILKDSVASSHIDIIGGHLYGVQPRNYPLALQKGKKLWMTEHYTDTDNANIWDKAMNVGLELHQSMVSNYSAYIWWYLRRSYGMLTEDGNISKRGYIMSQFSKFIRPGDVRIAATEVPESNVYVTAYKNRSGKLVIAVVNKTNSHKKLDFTLQNGTVGSMTKYVTSASQNVGYAGKYAVSNNRFTAYADPLSVQTFVSE

SEQ ID NO: 54 - protein, 382 aa, Vibrio rhizosphaerae; identical to SEQ ID NO: 53
  mat_peptide (1)..(382)

SEQ ID NO: 55 - DNA, 1254 bp, Artificial Sequence
  Synthetic construct
  CDS (1)..(1251)
  sig_peptide (1)..(81)
  mat_peptide (82)..(1251)
atgaagaaaccgttggggaaaattgtcgcaagcaccgcactactcatttctgttgcttttagttcatcgatagcatcagcacatcatcatcaccatcatcctaggggctcagtctatattaactttaacacggaatatcaagaaatcgacggctttggcgcaatgaatgcaccgggatgggttaatgatctgacatcagcacaagcaacaaaagcatttggcaatggcgacggccaaatgggcctgtcaattatgagaatgagaattgatccggatagcaatcagtggtatagacaagttccgacagcacaaattgcatattcatatggcgcaaaactgctggcaacaccgtggtcaccgcctgcatatatgaaaacaaataacaacgtcaacaacggtggcaaactgaaaaaagaacattattggggctatacggatcatctgatggactttacaaactacatggcaggcaaaaacgcaccgatttatgcactgtcaattcagaatgaaccggattggcatccgaattatgaatcatgcgattggtcaggcgcagactttgttaattatctgaatgatcaaggctggcgtctggattcatcactgaaaattctggcaccggaatcactgggctttaatccggcactgtcagatccgattctgaaagattcagttgcaagcagccatattgatattattggcggacatctgtatggcgttcaaccgagaaattatccgctggcactgcaaaaaggcaaaaaactgtggatgacggaacattatacagatacagataacgcgaacatctgggacaaagctatgaatgttggcctggaactgcatcaatcaatggtttcaaattatagcgcgtatatctggtggtatcttagaagatcatatggcatgctgacagaagatggcaatatttcaaaacgcggatatatcatgagccagtttagcaagtttattagaccgggtgatgttagaattgcagcaacggaagttccggaaagcaatgtttatgttacggcgtataaaaacagaagcggcaaactggttattgcggtcgtcaataaaacaaacagccataaaaaactggattttacgctgcaaaatggcacagttggcagcatgacaaaatatgttacaagcgcatcacagaatgtcggctatgcaggcaaatatgcagttagcaataatcgctttacagcatatgcagatccgctgtcagttcaaacatttgtttcagaataa

SEQ ID NO: 56 - protein, 417 aa, Artificial Sequence
  Synthetic Construct
MKKPLGKIVASTALLISVAFSSSIASAHHHHHHPRGSVYINFNTEYQEIDGFGAMNAPGWVNDLTSAQATKAFGNGDGQMGLSIMRMRIDPDSNQWYRQVPTAQIAYSYGAKLLATPWSPPAYMKTNNNVNNGGKLKKEHYWGYTDHLMDFTNYMAGKNAPIYALSIQNEPDWHPNYESCDWSGADFVNYLNDQGWRLDSSLKILAPESLGFNPALSDPILKDSVASSHIDIIGGHLYGVQPRNYPLALQKGKKLWMTEHYTDTDNANIWDKAMNVGLELHQSMVSNYSAYIWWYLRRSYGMLTEDGNISKRGYIMSQFSKFIRPGDVRIAATEVPESNVYVTAYKNRSGKLVIAVVNKTNSHKKLDFTLQNGTVGSMTKYVTSASQNVGYAGKYAVSNNRFTAYADPLSVQTFVSE

SEQ ID NO: 57 - protein, 390 aa, Artificial Sequence
  Synthetic construct
  mat_peptide (1)..(390)
HHHHHHPRGSVYINFNTEYQEIDGFGAMNAPGWVNDLTSAQATKAFGNGDGQMGLSIMRMRIDPDSNQWYRQVPTAQIAYSYGAKLLATPWSPPAYMKTNNNVNNGGKLKKEHYWGYTDHLMDFTNYMAGKNAPIYALSIQNEPDWHPNYESCDWSGADFVNYLNDQGWRLDSSLKILAPESLGFNPALSDPILKDSVASSHIDIIGGHLYGVQPRNYPLALQKGKKLWMTEHYTDTDNANIWDKAMNVGLELHQSMVSNYSAYIWWYLRRSYGMLTEDGNISKRGYIMSQFSKFIRPGDVRIAATEVPESNVYVTAYKNRSGKLVIAVVNKTNSHKKLDFTLQNGTVGSMTKYVTSASQNVGYAGKYAVSNNRFTAYADPLSVQTFVSE

SEQ ID NO: 58 - DNA, 1767 bp, Clostridium acetobutylicum
  CDS (1)..(1764)
  sig_peptide (1)..(93)
  mat_peptide (94)..(1764)
atgaaaaacaaaataaaaaaaatagttttatctgtagttacattttctatgatttgtttaccatttacagatggatttacatccattaaagcagcttcaaatgttatggttaatttagcctcaaaaaagcaagtaattcgtggttttggaggaatgaattctgtagcttgggctggtgatttgacagcagctcaaagagaaacagcctttggaaatggaaataatcaactaggtctttctgtagtaagaatatttgtagatgataataagaacaattggtataaagagttaccaacagctaaaagtgctattgcacatggatcgatagtctttgctacgccttggaaccctccaagttctatgacagaaacctttaatagaaatggtgaaaaagcaaaacgccttagatatgataaatatggtgactatgctaaatatcttaatgattttgtatcatatatgaagaataatggtgttaatttgtatgctatttctgtacaaaatgagcctgattatgggcgcgattggacttggtggacaccacaggaagtacttcgttttatgagagactatgcaggaagtattaactgcagggtaatgtcacctgagtcattttcttatcaaaaaaacatgtacgatcctatattaaatgatccaaaagctcttgcgaacatggatatccttgggactcacacttatggtactcaagttaaagattttccttatccactttttaaacaaaaagcagcaggaaaagatctttggatgacagaagtgtatgttccaaacagtgatgctaattcagcagatcgctggcctgaagcgttagaggttgcaaatcatataaacaatgctatggtagaaggcgatttccaagcgtatgtatggtggtatataagaagatcctatggattgatcaaagagaatggtgctataagtaaacgtggctacatgatggcccatttctccaagtttgttcgtcctggatatgtaagagttgatgcaacaaaaaatccagtaggtaatgtttatgtgtcagcctacacaggaaataataaagtggttattgttgcaataaacaaaggaacatatccagtaaatcaaagctttaatatacaaaatagtacggtatctaatgtgtcttcatgggtaacttcaggaacattaaatatggctaaaacaaattctaatataaatgcagctaatggaagattcaatgcatcacttccagctcaaagtgtaactacatttgtagcagatttaaattctactaagcctacaactaaccctacaactaatcctactcctggaagtactgtaactttaaataatggatggtactacattaaaaatattaatgctcaaaaataccttcaagttgcaaataatacaggaaaagcaggacaaaatgttgagcttggttcaggttcaggagttgctggacaaaaatggtatttaacaaatacaggagatggttatattactttaaaaaatgctttaggtaactatatgctggatgttagctatggtgaaaataaggatggaagtaatattcaaatttttaatgcatacagcggagattcacaaaaatttgctgtaaaagcttcttctaaaaatggtcaatattctgttgcaactaagagtagtaatggatctaaggtattagatgattataactttggaactgctgatggcactaatgtatgtcagtggacttatggaggaaatgctaatcaattatgggtatttgagcctactaacaactaa

SEQ ID NO: 59 - protein, 588 aa, Clostridium acetobutylicum
MKNKIKKIVLSVVTFSMICLPFTDGFTSIKAASNVMVNLASKKQVIRGFGGMNSVAWAGDLTAAQRETAFGNGNNQLGLSVVRIFVDDNKNNWYKELPTAKSAIAHGSIVFATPWNPPSSMTETFNRNGEKAKRLRYDKYGDYAKYLNDFVSYMKNNGVNLYAISVQNEPDYGRDWTWWTPQEVLRFMRDYAGSINCRVMSPESFSYQKNMYDPILNDPKALANMDILGTHTYGTQVKDFPYPLFKQKAAGKDLWMTEVYVPNSDANSADRWPEALEVANHINNAMVEGDFQAYVWWYIRRSYGLIKENGAISKRGYMMAHFSKFVRPGYVRVDATKNPVGNVYVSAYTGNNKVVIVAINKGTYPVNQSFNIQNSTVSNVSSWVTSGTLNMAKTNSNINAANGRFNASLPAQSVTTFVADLNSTKPTTNPTTNPTPGSTVTLNNGWYYIKNINAQKYLQVANNTGKAGQNVELGSGSGVAGQKWYLTNTGDGYITLKNALGNYMLDVSYGENKDGSNIQIFNAYSGDSQKFAVKASSKNGQYSVATKSSNGSKVLDDYNFGTADGTNVCQWTYGGNANQLWVFEPTNN

SEQ ID NO: 60 - protein, 557 aa, Clostridium acetobutylicum
  mat_peptide (1)..(557)
ASNVMVNLASKKQVIRGFGGMNSVAWAGDLTAAQRETAFGNGNNQLGLSVVRIFVDDNKNNWYKELPTAKSAIAHGSIVFATPWNPPSSMTETFNRNGEKAKRLRYDKYGDYAKYLNDFVSYMKNNGVNLYAISVQNEPDYGRDWTWWTPQEVLRFMRDYAGSINCRVMSPESFSYQKNMYDPILNDPKALANMDILGTHTYGTQVKDFPYPLFKQKAAGKDLWMTEVYVPNSDANSADRWPEALEVANHINNAMVEGDFQAYVWWYIRRSYGLIKENGAISKRGYMMAHFSKFVRPGYVRVDATKNPVGNVYVSAYTGNNKVVIVAINKGTYPVNQSFNIQNSTVSNVSSWVTSGTLNMAKTNSNINAANGRFNASLPAQSVTTFVADLNSTKPTTNPTTNPTPGSTVTLNNGWYYIKNINAQKYLQVANNTGKAGQNVELGSGSGVAGQKWYLTNTGDGYITLKNALGNYMLDVSYGENKDGSNIQIFNAYSGDSQKFAVKASSKNGQYSVATKSSNGSKVLDDYNFGTADGTNVCQWTYGGNANQLWVFEPTNN

SEQ ID NO: 61 - DNA, 1800 bp, Artificial Sequence
  Synthetic construct
  CDS (1)..(1800)
  sig_peptide (1)..(93)
  mat_peptide (94)..(1800)
atgaaaaacaaaataaaaaaaatagttttatctgtagttacattttctatgatttgtttaccatttacagatggatttacatccattaaagcagcttcaaatgttatggttaatttagcctcaaaaaagcaagtaattcgtggttttggaggaatgaattctgtagcttgggctggtgatttgacagcagctcaaagagaaacagcctttggaaatggaaataatcaactaggtctttctgtagtaagaatatttgtagatgataataagaacaattggtataaagagttaccaacagctaaaagtgctattgcacatggatcgatagtctttgctacgccttggaaccctccaagttctatgacagaaacctttaatagaaatggtgaaaaagcaaaacgccttagatatgataaatatggtgactatgctaaatatcttaatgattttgtatcatatatgaagaataatggtgttaatttgtatgctatttctgtacaaaatgagcctgattatgggcgcgattggacttggtggacaccacaggaagtacttcgttttatgagagactatgcaggaagtattaactgcagggtaatgtcacctgagtcattttcttatcaaaaaaacatgtacgatcctatattaaatgatccaaaagctcttgcgaacatggatatccttgggactcacacttatggtactcaagttaaagattttccttatccactttttaaacaaaaagcagcaggaaaagatctttggatgacagaagtgtatgttccaaacagtgatgctaattcagcagatcgctggcctgaagcgttagaggttgcaaatcatataaacaatgctatggtagaaggcgatttccaagcgtatgtatggtggtatataagaagatcctatggattgatcaaagagaatggtgctataagtaaacgtggctacatgatggcccatttctccaagtttgttcgtcctggatatgtaagagttgatgcaacaaaaaatccagtaggtaatgtttatgtgtcagcctacacaggaaataataaagtggttattgttgcaataaacaaaggaacatatccagtaaatcaaagctttaatatacaaaatagtacggtatctaatgtgtcttcatgggtaacttcaggaacattaaatatggctaaaacaaattctaatataaatgcagctaatggaagattcaatgcatcacttccagctcaaagtgtaactacatttgtagcagatttaaattctactaagcctacaactaaccctacaactaatcctactcctggaagtactgtaactttaaataatggatggtactacattaaaaatattaatgctcaaaaataccttcaagttgcaaataatacaggaaaagcaggacaaaatgttgagcttggttcaggttcaggagttgctggacaaaaatggtatttaacaaatacaggagatggttatattactttaaaaaatgctttaggtaactatatgctggatgttagctatggtgaaaataaggatggaagtaatattcaaatttttaatgcatacagcggagattcacaaaaatttgctgtaaaagcttcttctaaaaatggtcaatattctgttgcaactaagagtagtaatggatctaaggtattagatgattataactttggaactgctgatggcactaatgtatgtcagtggacttatggaggaaatgctaatcaattatgggtatttgagcctactaacaacgacccagctttcttgtacaaagtggttcgatcgcgt

SEQ ID NO: 62 - protein, 600 aa, Artificial Sequence
  Synthetic Construct
MKNKIKKIVLSVVTFSMICLPFTDGFTSIKAASNVMVNLASKKQVIRGFGGMNSVAWAGDLTAAQRETAFGNGNNQLGLSVVRIFVDDNKNNWYKELPTAKSAIAHGSIVFATPWNPPSSMTETFNRNGEKAKRLRYDKYGDYAKYLNDFVSYMKNNGVNLYAISVQNEPDYGRDWTWWTPQEVLRFMRDYAGSINCRVMSPESFSYQKNMYDPILNDPKALANMDILGTHTYGTQVKDFPYPLFKQKAAGKDLWMTEVYVPNSDANSADRWPEALEVANHINNAMVEGDFQAYVWWYIRRSYGLIKENGAISKRGYMMAHFSKFVRPGYVRVDATKNPVGNVYVSAYTGNNKVVIVAINKGTYPVNQSFNIQNSTVSNVSSWVTSGTLNMAKTNSNINAANGRFNASLPAQSVTTFVADLNSTKPTTNPTTNPTPGSTVTLNNGWYYIKNINAQKYLQVANNTGKAGQNVELGSGSGVAGQKWYLTNTGDGYITLKNALGNYMLDVSYGENKDGSNIQIFNAYSGDSQKFAVKASSKNGQYSVATKSSNGSKVLDDYNFGTADGTNVCQWTYGGNANQLWVFEPTNNDPAFLYKVVRSR

SEQ ID NO: 63 - protein, 569 aa, Artificial Sequence
  Synthetic construct
  mat_peptide (1)..(569)
ASNVMVNLASKKQVIRGFGGMNSVAWAGDLTAAQRETAFGNGNNQLGLSVVRIFVDDNKNNWYKELPTAKSAIAHGSIVFATPWNPPSSMTETFNRNGEKAKRLRYDKYGDYAKYLNDFVSYMKNNGVNLYAISVQNEPDYGRDWTWWTPQEVLRFMRDYAGSINCRVMSPESFSYQKNMYDPILNDPKALANMDILGTHTYGTQVKDFPYPLFKQKAAGKDLWMTEVYVPNSDANSADRWPEALEVANHINNAMVEGDFQAYVWWYIRRSYGLIKENGAISKRGYMMAHFSKFVRPGYVRVDATKNPVGNVYVSAYTGNNKVVIVAINKGTYPVNQSFNIQNSTVSNVSSWVTSGTLNMAKTNSNINAANGRFNASLPAQSVTTFVADLNSTKPTTNPTTNPTPGSTVTLNNGWYYIKNINAQKYLQVANNTGKAGQNVELGSGSGVAGQKWYLTNTGDGYITLKNALGNYMLDVSYGENKDGSNIQIFNAYSGDSQKFAVKASSKNGQYSVATKSSNGSKVLDDYNFGTADGTNVCQWTYGGNANQLWVFEPTNNDPAFLYKVVRSR

SEQ ID NO: 64 - protein, 461 aa, Scytalidium thermophilum
  mat_peptide (1)..(461)
ATVSPRQSGTITVNLGQTYQRIDGFGFSLAFQRANLITNMSDKTKQKELLDLLFNKTTGAGFTIIRNGIGSTPNSNSDFMNTIAPNNPGGPNATPQYVWDGKDSGQLWVTQQAVSLYGVKQVYANAWSAPGYMKTNGRDTNGGTLCGMPGASCASGDWRQAYANYLVQYIRFYQQSGVNITHLGFLNEPDYSASYASMQANGNQAADFIKILDATLRREGLSEQVGITCCDSMGWGNQANMMNAIRSQGAEGLLKAVTTHTYTGAPNGPVNYRAPLWFSEQCDLNGQWTTAWYAFGGAGEGLTWANNIFNAIVNNNASGYLWWEGVQWPNPNTNEKLIRVDNTTNTYEVSRRLWAFANWSRYVKPGAVRVGTSGAPGGLRVAAFRNEDGTIAVVAISSSGSAVNVSIRLSGGGVGGASAVRAYVSDNTRNCENTPASVGADGSITGSVAARSITTFYILPA

(19) United States
(12) Patent Application Publication
(10) Pub. No.: US 2019/0194635 A1
(43) Pub. Date: Jun. 27, 2019

(54) Polypeptides Having Xylanase Activity and Polynucleotides Encoding Same

(71) Applicant: Novozymes A/S, Bagsvaerd (DK)

(72) Inventors: Soren Nymand-Grarup, Aabyhoej (DK); Ninfa Rangel Pedersen, Soeborg (DK); Lorena Palmen Gonzalez, Malmo (SE); Dan Pettersson, Lynge (DK); Jens Magnus Eklof, FrbC (DK); Charlotte Blom, Lynge (DK); Jesper Salomon, Holte (DK)

(73) Assignee: Novozymes A/S, Bagsvaerd (DE)

(21) Appl. No.: 15/780,299

(22) PCT Filed: Dec. 16, 2016
(86) PCT No.: PCT/EP2016/081511

§ 371 Date: May 31, 2018

(30) Foreign Application Priority Data
Dec. 18, 2015 (EP) EP15201315.7

(51) Int. Cl. C12N 9/24; C12P 19/02; A23K 20/189; A23K 20/174; A23K 50/30; A23K 50/75

(52) U.S. Cl. CPC C12N 9/2482; C12P 19/02; A23K 20/189; C12Y 302/01008; A23K 50/30; A23K 50/75; A23K 20/174

(57) ABSTRACT

The present invention relates to polypeptides having xylanase activity and polynucleotides encoding the polypeptides. The invention also relates to nucleic acid constructs, vectors, and host cells comprising the polynucleotides as well as methods of producing and using the polypeptides. The invention also relates to compositions comprising the polypeptides of the invention and the use of the polypeptides of the invention to solubilise xylan and in animal feed.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to polypeptides having xylanase activity and polynucleotides encoding the polypeptides. The invention also relates to nucleic acid constructs, vectors, and host cells comprising the polynucleotides as well as methods of producing and using the polypeptides. The invention also relates to compositions comprising the polypeptides of the invention and the use of the polypeptides of the invention to solubilise xylan and in animal feed.

### Description of the Related Art

Xylans are hemicelluloses found in all land plants (Popper and Tuohy, Plant Physiology, 2010, 153:373-383). They are especially abundant in secondary cell walls and xylem cells. In grasses, with type II cell walls, glucurono arabinoxylans are the main hemicellulose and are present as soluble or insoluble dietary fiber in many grass based food and feed products.

Plant xylans have a 13-1,4-linked xylopyranose backbone that can be substituted at the O2 or O3 position with arabinose, glucuronic acid and acetic acid in a species and tissue specific manner. The starch-rich seeds of the sub-family Panicoideae with economically important species such as corn, sorghum, rice and millet have special types of highly substituted xylans in their cell walls. Compared to wheat flour, wherein over 60% of the xylosyl units in the arabinoxylan backbone are unsubstituted. In corn kernel xylan, the corresponding percentage of unsubstituted backbone xylosyls is 20-30%, and in sorghum it is 35-40% (Huismann et al. Carbohydrate Polymers, 2000, 42:269-279). Furthermore, in corn and sorghum the xylan side chains can be longer than a single arabinose or glucuronic acid substitution which is common in other xylans. This added side chain complexity is often due to L- and D-galactose and D-xylose sugars bound to the side chain arabinose or glucuronic acid. About every tenth arabinose in corn kernel xylan is also esterified with a ferulic acid and about every fourth xylose carries an acetylation (Agger et al. J. Agric. Food Chem, 2010, 58:6141-6148). All of these factors combined make the highly substituted xylans in corn and sorghum resistant to degradation by traditional xylanases.

The known enzymes responsible for the hydrolysis of the xylan backbone are classified into enzyme families based on sequence similarity (www.cazy.org). The enzymes with mainly endo-xylanase activity have previously been described in Glycoside hydrolase family (GH) 5, 8, 10, 11, 30 and 98. The enzymes within a family share some characteristics such as 3D fold and they usually share the same reaction mechanism. Some GH families have narrow or mono-specific substrate specificities while other families have broad substrate specificities.

Commercially available GH10 and GH11 xylanases are often used to break down the xylose backbone of arabinoxylan. In animal feed this results in a degradation of the cereal cell wall with a subsequent improvement in nutrient release (starch and protein) encapsulated within the cells. Degradation of xylan also results in the formation of xylose oligomers that may be utilised for hind gut fermentation and therefore can help an animal to obtain more digestible energy. However, such xylanases are sensitive to side chain steric hindrance and whilst they are effective at degrading arabinoxylan from wheat, they are not very effective on the xylan found in the seeds of Poaceae species, such as corn or sorghum. Corn is used around the world in animal feed and thus there is a need to discover new polypeptides having xylanase activity that are capable of breaking down the highly branched xylan backbone in the cell wall in order to release more xylose and other nutrients which are trapped inside the cell wall.

WO 2013/067964 relates to GH30 subfamily 7 xylanases for biomass conversion. However, in a typical biomass conversion process, the biomass is first pre-treated under acidic or alkaline conditions in order to partially degrade the biomass (e.g. remove side chain sugars such as arabinose) before the enzymatic process. WO 2011/038019 relates to novel glycosyl hydrolases and the use thereof in the saccharification of cellulosic and hemicellulosic materials into sugars (i.e. biomass conversion). Two GH30 subfamily 7 xylanases are disclosed; however, the corncobs are pre-treated with ammonia before a 6-8 enzymatic combination is added to degrade the biomass.

As is demonstrated herein, GH30 subfamily 7 xylanases are unable to degrade the xylose backbone of sterically hindered arabinoxylan which has not been pre-treated. Thus the objective of this invention is to provide xylanases which are able to solubilise this highly branched xylan backbone found in untreated maize.

### SUMMARY OF THE INVENTION

The present invention relates to a method of solubilising xylan from plant based material, comprising treating plant based material with a GH30 subfamily 8 polypeptide having xylanase activity, wherein the GH30 subfamily 8 polypeptide having xylanase activity is selected from the group consisting of:
(a) a polypeptide having at least 80% sequence identity to the polypeptide of SEQ ID NO: 60;
(b) a polypeptide having at least 80% sequence identity to the polypeptide of SEQ ID NO: 6;
(c) a polypeptide having at least 92% sequence identity to the polypeptide of SEQ ID NO: 12;
(d) a polypeptide having at least 80% sequence identity to the polypeptide of SEQ ID NO: 18;
(e) a polypeptide having at least 80% sequence identity to the polypeptide of SEQ ID NO: 24;
(f) a polypeptide having at least 80% sequence identity to the polypeptide of SEQ ID NO: 30;
(g) a polypeptide having at least 80% sequence identity to the polypeptide of SEQ ID NO: 36;
(h) a polypeptide having at least 85% sequence identity to the polypeptide of SEQ ID NO: 42;
(i) a polypeptide having at least 80% sequence identity to the polypeptide of SEQ ID NO: 48;
(j) a polypeptide having at least 80% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (I) and a N-terminal and/or C-terminal His-tag and/or HQ-tag; and
(n) a fragment of a polypeptide of (a), (b), (c), (d), (e), (f), (g), (h), (i), (j), (k), (I) or (m) having at least 90% of the length of the mature polypeptide.

The present application also relates to granules comprising the xylanase of the invention and isolated polypeptides having xylanase activity as defined in the claims. The present application further relates to compositions comprising polypeptides having xylanase activity, animal feed and animal feed additives comprising the polypeptide(s) of the invention, methods of improving one or more performance parameters of an animal comprising administering to the animal the polypeptide or composition of the invention; methods of preparing an animal feed comprising mixing the polypeptide or composition of the invention with plant based material from the sub-family Panicoideae; methods for improving the nutritional value of an animal feed comprising mixing plant based material from the sub-family Panicoideae with the polypeptide or composition of the invention; use of the polypeptide or composition of the invention in animal feed, in animal feed additives, in the preparation of a composition for use in animal feed, for improving the nutritional value of an animal feed, for increasing digestibility of the animal feed, for improving one or more performance parameters in an animal, for solubilising xylan from plant based material of the sub-family Panicoideae, and/or for releasing starch from plant based material of the sub-family Panicoideae, polynucleotides encoding the polypeptides of the present invention; nucleic acid constructs; expression vectors; recombinant host cells comprising the polynucleotides; and methods of producing the polypeptides.

### Overview of Sequence Listing

SEQ ID NO: 1 is the motif YXWWY[I/L]RRXYG

SEQ ID NO: 2 is the Bacillus clausii secretion signal.

SEQ ID NO: 3 is the sequence of the His-tag.

SEQ ID NO: 4 is the gene sequence of the GH30_8 xylanase as isolated from Pseudoalteromonas tetraodonis.

SEQ ID NO: 5 is the amino acid sequence as deduced from SEQ ID NO: 4.

SEQ ID NO: 6 is the amino acid sequence of the mature GH30_8 xylanase from Pseudoalteromonas tetraodonis.

SEQ ID NO: 7 is the DNA sequence of the recombinant expressed DNA sequence from SEQ ID NO: 4 with His-tag and Savinase signal peptide.

SEQ ID NO: 8 is the amino acid sequence as deduced from SEQ ID NO: 7.

SEQ ID NO: 9 is the amino acid sequence of the mature GH30_8 xylanase obtained from SEQ ID NO: 7.

SEQ ID NO: 10 is the gene sequence of the GH30_8 xylanase as isolated from Paenibacillus sp-19179.

SEQ ID NO: 11 is the amino acid sequence as deduced from SEQ ID NO: 10.

SEQ ID NO: 12 is the amino acid sequence of the mature GH30_8 xylanase from Paenibacillus sp-19179.

SEQ ID NO: 13 is the DNA sequence of the recombinant expressed DNA sequence from SEQ ID NO: 10 with His-tag and Savinase signal peptide.

SEQ ID NO: 14 is the amino acid sequence as deduced from SEQ ID NO: 13.

SEQ ID NO: 15 is the amino acid sequence of the mature GH30_8 xylanase obtained from SEQ ID NO: 13.

SEQ ID NO: 16 is the gene sequence of the GH30_8 xylanase as isolated from Pectobacterium carotovorum subsp. carotovorum.

SEQ ID NO: 17 is the amino acid sequence as deduced from SEQ ID NO: 16.

SEQ ID NO: 18 is the amino acid sequence of the mature GH30_8 xylanase Pectobacterium carotovorum subsp. carotovorum.

SEQ ID NO: 19 is the DNA sequence of the recombinant expressed DNA sequence from SEQ ID NO: 16 with His-tag and Savinase signal peptide.

SEQ ID NO: 20 is the amino acid sequence as deduced from SEQ ID NO: 19.

SEQ ID NO: 21 is the amino acid sequence of the mature GH30_8 xylanase obtained from SEQ ID NO: 19.

SEQ ID NO: 22 is the gene sequence of the truncated GH30_8 xylanase as isolated from Ruminococcus sp. CAG:330.

SEQ ID NO: 23 is the amino acid sequence as deduced from SEQ ID NO: 22.

SEQ ID NO: 24 is the amino acid sequence of the mature GH30_8 xylanase Ruminococcus sp. CAG:330.

SEQ ID NO: 25 is the DNA sequence of the recombinant expressed DNA sequence from SEQ ID NO: 22 with His-tag and Savinase signal peptide.

SEQ ID NO: 26 is the amino acid sequence as deduced from SEQ ID NO: 25.

SEQ ID NO: 27 is the amino acid sequence of the mature GH30_8 xylanase obtained from SEQ ID NO: 25.

SEQ ID NO: 28 is the gene sequence of the GH30_8 xylanase as isolated from Streptomyces sp-62627.

SEQ ID NO: 29 is the amino acid sequence as deduced from SEQ ID NO: 28.

SEQ ID NO: 30 is the amino acid sequence of the mature GH30_8 xylanase Streptomyces sp-62627.

SEQ ID NO: 31 is the DNA sequence of the recombinant expressed DNA sequence from SEQ ID NO: 28 with His-tag and Savinase signal peptide.

SEQ ID NO: 32 is the amino acid sequence as deduced from SEQ ID NO: 31.

SEQ ID NO: 33 is the amino acid sequence of the mature GH30_8 xylanase obtained from SEQ ID NO: 31.

SEQ ID NO: 34 is the gene sequence of the GH30_8 xylanase as isolated from Clostridium saccharobutylicum.

SEQ ID NO: 35 is the amino acid sequence as deduced from SEQ ID NO: 34.

SEQ ID NO: 36 is the amino acid sequence of the mature GH30_8 xylanase Clostridium saccharobutylicum.

SEQ ID NO: 37 is the DNA sequence of the recombinant expressed DNA sequence from SEQ ID NO: 34 with His-tag and Savinase signal peptide.

SEQ ID NO: 38 is the amino acid sequence as deduced from SEQ ID NO: 37.

SEQ ID NO: 39 is the amino acid sequence of the mature GH30_8 xylanase obtained from SEQ ID NO: 37.

SEQ ID NO: 40 is the gene sequence of the GH30_8 xylanase as isolated from Paenibacillus panacisoli.

SEQ ID NO: 41 is the amino acid sequence as deduced from SEQ ID NO: 40.

SEQ ID NO: 42 is the amino acid sequence of the mature GH30_8 xylanase Paenibacillus panacisoli.

SEQ ID NO: 43 is the DNA sequence of the recombinant expressed DNA sequence from SEQ ID NO: 40 with His-tag and Savinase signal peptide.

SEQ ID NO: 44 is the amino acid sequence as deduced from SEQ ID NO: 43.

SEQ ID NO: 45 is the amino acid sequence of the mature GH30_8 xylanase obtained from SEQ ID NO: 43.

SEQ ID NO: 46 is the gene sequence of the truncated GH30_8 xylanase as isolated from Human Stool metagenome.

SEQ ID NO: 47 is the amino acid sequence as deduced from SEQ ID NO: 46.

SEQ ID NO: 48 is the amino acid sequence of the mature GH30_8 xylanase Human Stool metagenome.

SEQ ID NO: 49 is the DNA sequence of the recombinant expressed DNA sequence from SEQ ID NO: 46 with His-tag and Savinase signal peptide.

SEQ ID NO: 50 is the amino acid sequence as deduced from SEQ ID NO: 49.

SEQ ID NO: 51 is the amino acid sequence of the mature GH30_8 xylanase obtained from SEQ ID NO: 49.

SEQ ID NO: 52 is the gene sequence of the GH30_8 xylanase as isolated from Vibrio rhizosphaerae.

SEQ ID NO: 53 is the amino acid sequence as deduced from SEQ ID NO: 52.

SEQ ID NO: 54 is the amino acid sequence of the mature GH30_8 xylanase Vibrio rhizosphaerae.

SEQ ID NO: 55 is the DNA sequence of the recombinant expressed DNA sequence from SEQ ID NO: 52 with His-tag and Savinase signal peptide.

SEQ ID NO: 56 is the amino acid sequence as deduced from SEQ ID NO: 55.

SEQ ID NO: 57 is the amino acid sequence of the mature GH30_8 xylanase obtained from SEQ ID NO: 55.

SEQ ID NO: 58 is the gene sequence of the GH30_8 xylanase as isolated from Clostridium acetobutylicum.

SEQ ID NO: 59 is the amino acid sequence as deduced from SEQ ID NO: 58.

SEQ ID NO: 60 is the amino acid sequence of the mature GH30_8 xylanase from Clostridium acetobutylicum.

SEQ ID NO: 61 is the gene sequence of SEQ ID NO: 58 with C-terminal tail.

SEQ ID NO: 62 is the amino acid sequence as deduced from SEQ ID NO: 61.

SEQ ID NO: 63 is the amino acid sequence of the mature GH30_8 xylanase obtained from SEQ ID NO: 61.

SEQ ID NO: 64 is the amino acid sequence of the mature GH30_7 xylanase from Scytalidium thermophilum as disclosed as SEQ ID NO: 4 of WO 2013/067964.

### Definitions

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Animal: The term “animal” refers to all animals except humans. Examples of animals are non-ruminants, and ruminants. Ruminant animals include, for example, animals such as sheep, goats, cattle, e.g. beef cattle, cows, and young calves, deer, yank, camel, llama and kangaroo. Non-ruminant animals include mono-gastric animals, e.g. pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), young calves; fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

Animal feed: The term “animal feed” refers to any compound, preparation, or mixture suitable for, or intended for intake by an animal. Animal feed for a mono-gastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Arabinoxylan-containing material: The term “Arabinoxylan-containing material” means any material containing arabinoxylan. Arabinoxylan is a hemicellulose found in both the primary and secondary cell walls of plants, including woods and cereal grains, consisting of copolymers of two pentose sugars, arabinose and xylose. The arabinoxylan chain contains a large number of 1,4-linked xylose units. Many xylose units are substituted with 2-, 3- or 2,3-substituted arabinose residues.

Examples of arabinoxylan-containing material are forage, roughage, seeds and grains (either whole or prepared by crushing, milling, etc from e.g. corn, oats, rye, barley, wheat), trees or hard woods (such as poplar, willow, eucalyptus, palm, maple, birch), bamboo, herbaceous and/or woody energy crops, agricultural food and feed crops, animal feed products, cassava peels, cocoa pods, sugar cane, sugar beet, locust bean pulp, vegetable or fruit pomaces, wood waste, bark, shavings, sawdust, wood pulp, pulping liquor, waste paper, cardboard, construction and demolition wood waste, industrial or municipal waste water solids or sludge, manure, by-product from brewing and/or fermentation processes, wet distillers grain, dried distillers grain, spent grain, vinasse and bagasse.

Forage as defined herein also includes roughage. Forage is fresh plant material such as hay and silage from forage plants, grass and other forage plants, grass and other forage plants, seaweed, sprouted grains and legumes, or any combination thereof. Examples of forage plants are Alfalfa (Lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, miscanthus, orchard grass, ryegrass, switchgrass, Timothy-grass), corn (maize), hemp, millet, barley, oats, rye, sorghum, soybeans and wheat and vegetables such as beets. Crops suitable for ensilage are the ordinary grasses, clovers, alfalfa, vetches, oats, rye and maize. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Roughage is generally dry plant material with high levels of fiber, such as fiber, bran, husks from seeds and grains and crop residues (such as stover, copra, straw, chaff, sugar beet waste).

Preferred sources of arabinoxylan-containing materials are forage, roughage, seeds and grains, sugar cane, sugar beet and wood pulp.

Body Weight Gain: The term “body weight gain” means an increase in live weight of an animal during a given period of time e.g. the increase in weight from day 1 to day 21.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Feed Conversion Ratio: The term “feed conversion ratio” the amount of feed fed to an animal to increase the weight of the animal by a specified amount. An improved feed conversion ratio means a lower feed conversion ratio. By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Fragment: The term “fragment” means a polypeptide having one or more (e.g., several) amino acids absent from the amino and/or carboxyl terminus of a mature polypeptide or domain; wherein the fragment has xylanase activity.

In one aspect, a fragment contains at least 90% of the amino acids of the mature polypeptide, such as 502 amino acids (SEQ ID NO: 59 or 60), 512 amino acids (SEQ ID NO: 52 or 63), 344 amino acids (SEQ ID NO: 5 or 6), 351 amino acids (SEQ ID NO: 8 or 9), 352 amino acids (SEQ ID NO: 11 or 12), 360 amino acids (SEQ ID NO: 14 or 15), 345 amino acids (SEQ ID NO: 17 or 18), 352 amino acids (SEQ ID NO: 20 or 21), 509 amino acids (SEQ ID NO: 23 or 24), 516 amino acids (SEQ ID NO: 26 or 27), 357 amino acids (SEQ ID NO: 29 or 30), 364 amino acids (SEQ ID NO: 32 or 33), 353 amino acids (SEQ ID NO: 35 or 36), 360 amino acids (SEQ ID NO: 38 or 39), 372 amino acids (SEQ ID NO: 41 or 42), 379 amino acids (SEQ ID NO: 44 or 45), 359 amino acids (SEQ ID NO: 47 or 48), 366 amino acids (SEQ ID NO: 50 or 51), 344 amino acids (SEQ ID NO: 53 or 54) or 351 amino acids (SEQ ID NO: 56 or 57).

In another aspect, a fragment contains at least 92% of the amino acids of the mature polypeptide, such as 513 amino acids (SEQ ID NO: 59 or 60), 523 amino acids (SEQ ID NO: 52 or 63), 352 amino acids (SEQ ID NO: 5 or 6), 359 amino acids (SEQ ID NO: 8 or 9), 360 amino acids (SEQ ID NO: 11 or 12), 368 amino acids (SEQ ID NO: 14 or 15), 353 amino acids (SEQ ID NO: 17 or 18), 360 amino acids (SEQ ID NO: 20 or 21), 520 amino acids (SEQ ID NO: 23 or 24), 528 amino acids (SEQ ID NO: 26 or 27), 365 amino acids (SEQ ID NO: 29 or 30), 372 amino acids (SEQ ID NO: 32 or 33), 361 amino acids (SEQ ID NO: 35 or 36), 368 amino acids (SEQ ID NO: 38 or 39), 380 amino acids (SEQ ID NO: 41 or 42), 388 amino acids (SEQ ID NO: 44 or 45), 367 amino acids (SEQ ID NO: 47 or 48), 374 amino acids (SEQ ID NO: 50 or 51), 352 amino acids (SEQ ID NO: 53 or 54) or 359 amino acids (SEQ ID NO: 56 or 57).

In another aspect, a fragment contains at least 94% of the amino acids of the mature polypeptide, such as 524 amino acids (SEQ ID NO: 59 or 60), 534 amino acids (SEQ ID NO: 52 or 63), 360 amino acids (SEQ ID NO: 5 or 6), 367 amino acids (SEQ ID NO: 8 or 9), 368 amino acids (SEQ ID NO: 11 or 12), 376 amino acids (SEQ ID NO: 14 or 15), 361 amino acids (SEQ ID NO: 17 or 18), 368 amino acids (SEQ ID NO: 20 or 21), 532 amino acids (SEQ ID NO: 23 or 24), 539 amino acids (SEQ ID NO: 26 or 27), 373 amino acids (SEQ ID NO: 29 or 30), 380 amino acids (SEQ ID NO: 32 or 33), 369 amino acids (SEQ ID NO: 35 or 36), 376 amino acids (SEQ ID NO: 38 or 39), 389 amino acids (SEQ ID NO: 41 or 42), 396 amino acids (SEQ ID NO: 44 or 45), 375 amino acids (SEQ ID NO: 47 or 48), 382 amino acids (SEQ ID NO: 50 or 51), 360 amino acids (SEQ ID NO: 53 or 54) or 367 amino acids (SEQ ID NO: 56 or 57).

In another aspect, a fragment contains at least 96% of the amino acids of the mature polypeptide, such as 535 amino acids (SEQ ID NO: 59 or 60), 546 amino acids (SEQ ID NO: 52 or 63), 367 amino acids (SEQ ID NO: 5 or 6), 375 amino acids (SEQ ID NO: 8 or 9), 376 amino acids (SEQ ID NO: 11 or 12), 384 amino acids (SEQ ID NO: 14 or 15), 368 amino acids (SEQ ID NO: 17 or 18), 376 amino acids (SEQ ID NO: 20 or 21), 543 amino acids (SEQ ID NO: 23 or 24), 551 amino acids (SEQ ID NO: 26 or 27), 381 amino acids (SEQ ID NO: 29 or 30), 388 amino acids (SEQ ID NO: 32 or 33), 377 amino acids (SEQ ID NO: 35 or 36), 384 amino acids (SEQ ID NO: 38 or 39), 397 amino acids (SEQ ID NO: 41 or 42), 405 amino acids (SEQ ID NO: 44 or 45), 383 amino acids (SEQ ID NO: 47 or 48), 390 amino acids (SEQ ID NO: 50 or 51), 367 amino acids (SEQ ID NO: 53 or 54) or 375 amino acids (SEQ ID NO: 56 or 57).

In another aspect, a fragment contains at least 98% of the amino acids of the mature polypeptide, such as 546 amino acids (SEQ ID NO: 59 or 60), 557 amino acids (SEQ ID NO: 52 or 63), 375 amino acids (SEQ ID NO: 5 or 6), 383 amino acids (SEQ ID NO: 8 or 9), 384 amino acids (SEQ ID NO: 11 or 12), 392 amino acids (SEQ ID NO: 14 or 15), 376 amino acids (SEQ ID NO: 17 or 18), 384 amino acids (SEQ ID NO: 20 or 21), 554 amino acids (SEQ ID NO: 23 or 24), 562 amino acids (SEQ ID NO: 26 or 27), 389 amino acids (SEQ ID NO: 29 or 30), 396 amino acids (SEQ ID NO: 32 or 33), 385 amino acids (SEQ ID NO: 35 or 36), 392 amino acids (SEQ ID NO: 38 or 39), 405 amino acids (SEQ ID NO: 41 or 42), 413 amino acids (SEQ ID NO: 44 or 45), 391 amino acids (SEQ ID NO: 47 or 48), 398 amino acids (SEQ ID NO: 50 or 51), 375 amino acids (SEQ ID NO: 53 or 54) or 383 amino acids (SEQ ID NO: 56 or 57).

In another aspect, a fragment contains at least 99% of the amino acids of the mature polypeptide, such as 552 amino acids (SEQ ID NO: 59 or 60), 563 amino acids (SEQ ID NO: 52 or 63), 379 amino acids (SEQ ID NO: 5 or 6), 387 amino acids (SEQ ID NO: 8 or 9), 388 amino acids (SEQ ID NO: 11 or 12), 396 amino acids (SEQ ID NO: 14 or 15), 380 amino acids (SEQ ID NO: 17 or 18), 388 amino acids (SEQ ID NO: 20 or 21), 560 amino acids (SEQ ID NO: 23 or 24), 568 amino acids (SEQ ID NO: 26 or 27), 393 amino acids (SEQ ID NO: 29 or 30), 400 amino acids (SEQ ID NO: 32 or 33), 389 amino acids (SEQ ID NO: 35 or 36), 396 amino acids (SEQ ID NO: 38 or 39), 409 amino acids (SEQ ID NO: 41 or 42), 417 amino acids (SEQ ID NO: 44 or 45), 395 amino acids (SEQ ID NO: 47 or 48), 402 amino acids (SEQ ID NO: 50 or 51), 379 amino acids (SEQ ID NO: 53 or 54) or 387 amino acids (SEQ ID NO: 56 or 57).

Highly branched xylan: The term “highly branched xylan” means that more than 50% of xylosyl units in the arabinoxylan backbone are substituted. This is preferably calculated from linkage analysis as performed in Huismann et al. Carbohydrate Polymers, 2000, 42:269-279.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc.

In one aspect, the mature polypeptide is amino acids 1 to 557 of SEQ ID NO: 59 based on the prediction program SignalP (Nielsen et al., 1997, Protein Engineering 10: 1-6)] that predicts amino acids −31 to −1 of SEQ ID NO: 59 are a signal peptide. In another aspect, the mature polypeptide is amino acids 1 to 557 of SEQ ID NO: 60. In one aspect, the mature polypeptide is amino acids 1 to 569 of SEQ ID NO: 62 and amino acids -31 to -1 of SEQ ID NO: 62 are a signal peptide. In another aspect, the mature polypeptide is amino acids 1 to 569 of SEQ ID NO: 63.

In one aspect, the mature polypeptide is amino acids 1 to 382 of SEQ ID NO: 5 based on the prediction program SignalP (Nielsen et al., supra)] that predicts amino acids −24 to −1 of SEQ ID NO: 5 are a signal peptide. In another aspect, the mature polypeptide is amino acids 1 to 382 of SEQ ID NO: 6. In one aspect, the mature polypeptide is amino acids 1 to 390 of SEQ ID NO: 8 and amino acids −27 to −1 of SEQ ID NO: 8 are a signal peptide. In another aspect, the mature polypeptide is amino acids 1 to 390 of SEQ ID NO: 9.

In one aspect, the mature polypeptide is amino acids 1 to 391 of SEQ ID NO: 11 based on the prediction program SignalP (Nielsen et al., supra)] that predicts amino acids −30 to −1 of SEQ ID NO: 11 are a signal peptide. In another aspect, the mature polypeptide is amino acids 1 to 391 of SEQ ID NO: 12. In one aspect, the mature polypeptide is amino acids 1 to 399 of SEQ ID NO: 14 and amino acids −27 to −1 of SEQ ID NO: 14 are a signal peptide. In another aspect, the mature polypeptide is amino acids 1 to 399 of SEQ ID NO: 15.

In one aspect, the mature polypeptide is amino acids 1 to 383 of SEQ ID NO: 17 based on the prediction program SignalP (Nielsen et al., supra)] that predicts amino acids −30 to −1 of SEQ ID NO: 17 are a signal peptide. In another aspect, the mature polypeptide is amino acids 1 to 383 of SEQ ID NO: 18. In one aspect, the mature polypeptide is amino acids 1 to 391 of SEQ ID NO: 20 and amino acids −27 to −1 of SEQ ID NO: 20 are a signal peptide. In another aspect, the mature polypeptide is amino acids 1 to 391 of SEQ ID NO: 21.

In one aspect, the mature polypeptide is amino acids 1 to 565 of SEQ ID NO: 23 based on the prediction program SignalP (Nielsen et al., supra)] that predicts amino acids −30 to −1 of SEQ ID NO: 23 are a signal peptide. In another aspect, the mature polypeptide is amino acids 1 to 565 of SEQ ID NO: 24. In one aspect, the mature polypeptide is amino acids 1 to 573 of SEQ ID NO: 26 and amino acids −27 to −1 of SEQ ID NO: 26 are a signal peptide. In another aspect, the mature polypeptide is amino acids 1 to 573 of SEQ ID NO: 27.

In one aspect, the mature polypeptide is amino acids 1 to 396 of SEQ ID NO: 29 based on the prediction program SignalP (Nielsen et al., supra)] that predicts amino acids −40 to −1 of SEQ ID NO: 29 are a signal peptide. In another aspect, the mature polypeptide is amino acids 1 to 396 of SEQ ID NO: 30. In one aspect, the mature polypeptide is amino acids 1 to 404 of SEQ ID NO: 32 and amino acids −27 to −1 of SEQ ID NO: 32 are a signal peptide. In another aspect, the mature polypeptide is amino acids 1 to 404 of SEQ ID NO: 33.

In one aspect, the mature polypeptide is amino acids 1 to 392 of SEQ ID NO: 35 based on the prediction program SignalP (Nielsen et al., supra)] that predicts amino acids −31 to −1 of SEQ ID NO: 35 are a signal peptide. In another aspect, the mature polypeptide is amino acids 1 to 392 of SEQ ID NO: 36. In one aspect, the mature polypeptide is amino acids 1 to 400 of SEQ ID NO: 38 and amino acids −27 to −1 of SEQ ID NO: 38 are a signal peptide. In another aspect, the mature polypeptide is amino acids 1 to 400 of SEQ ID NO: 39.

In one aspect, the mature polypeptide is amino acids 1 to 413 of SEQ ID NO: 41 based on the prediction program SignalP (Nielsen et al., supra)] that predicts amino acids −33 to −1 of SEQ ID NO: 41 are a signal peptide. In another aspect, the mature polypeptide is amino acids 1 to 413 of SEQ ID NO: 42. In one aspect, the mature polypeptide is amino acids 1 to 421 of SEQ

ID NO: 44 and amino acids −27 to −1 of SEQ ID NO: 44 are a signal peptide. In another aspect, the mature polypeptide is amino acids 1 to 421 of SEQ ID NO: 45.

In one aspect, the mature polypeptide is amino acids 1 to 398 of SEQ ID NO: 47 based on the prediction program SignalP (Nielsen et al., supra)] that predicts amino acids 0 to −1 of SEQ ID NO: 47 are a signal peptide. In another aspect, the mature polypeptide is amino acids 1 to 398 of SEQ ID NO: 48. In one aspect, the mature polypeptide is amino acids 1 to 406 of SEQ ID NO: 50 and amino acids −27 to −1 of SEQ ID NO: 50 are a signal peptide. In another aspect, the mature polypeptide is amino acids 1 to 406 of SEQ ID NO: 51.

In one aspect, the mature polypeptide is amino acids 1 to 382 of SEQ ID NO: 53 based on the prediction program SignalP (Nielsen et al., supra)] that predicts amino acids 0 to −1 of SEQ ID NO: 53 are a signal peptide. In another aspect, the mature polypeptide is amino acids 1 to 382 of SEQ ID NO: 54. In one aspect, the mature polypeptide is amino acids 1 to 390 of SEQ ID NO: 56 and amino acids −27 to −1 of SEQ ID NO: 56 are a signal peptide. In another aspect, the mature polypeptide is amino acids 1 to 390 of SEQ ID NO: 57.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having xylanase activity.

In one aspect, the mature polypeptide coding sequence is nucleotides 94 to 1764 of SEQ ID NO: 58 based on the prediction program SignalP (Nielsen et al., 1997, supra) that predicts nucleotides 1 to 93 of SEQ ID NO: 58 encode a signal peptide.

In one aspect, the mature polypeptide coding sequence is nucleotides 73 to 1218 of SEQ ID NO: 4 based on the prediction program SignalP (Nielsen et al., 1997, supra) that predicts nucleotides 1 to 72 of SEQ ID NO: 4 encode a signal peptide.

In one aspect, the mature polypeptide coding sequence is nucleotides 91 to 1263 of SEQ ID NO: 10 based on the prediction program SignalP (Nielsen et al., 1997, supra) that predicts nucleotides 1 to 90 of SEQ ID NO: 10 encode a signal peptide.

In one aspect, the mature polypeptide coding sequence is nucleotides 91 to 1239 of SEQ ID NO: 16 based on the prediction program SignalP (Nielsen et al., 1997, supra) that predicts nucleotides 1 to 90 of SEQ ID NO: 16 encode a signal peptide.

In one aspect, the mature polypeptide coding sequence is nucleotides 91 to 1785 of SEQ ID NO: 22 based on the prediction program SignalP (Nielsen et al., 1997, supra) that predicts nucleotides 1 to 90 of SEQ ID NO: 22 encode a signal peptide.

In one aspect, the mature polypeptide coding sequence is nucleotides 121 to 1308 of SEQ ID NO: 28 based on the prediction program SignalP (Nielsen et al., 1997, supra) that predicts nucleotides 1 to 120 of SEQ ID NO: 28 encode a signal peptide.

In one aspect, the mature polypeptide coding sequence is nucleotides 94 to 1269 of SEQ ID NO: 34 based on the prediction program SignalP (Nielsen et al., 1997, supra) that predicts nucleotides 1 to 93 of SEQ ID NO: 34 encode a signal peptide.

In one aspect, the mature polypeptide coding sequence is nucleotides 100 to 1338 of SEQ ID NO: 40 based on the prediction program SignalP (Nielsen et al., 1997, supra) that predicts nucleotides 1 to 99 of SEQ ID NO: 40 encode a signal peptide.

In one aspect, the mature polypeptide coding sequence is nucleotides 1 to 1194 of SEQ ID NO: 46.

In one aspect, the mature polypeptide coding sequence is nucleotides 1 to 1146 of SEQ ID NO: 52.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Nutrient Digestibility: The term “nutrient digestibility” means the fraction of a nutrient that disappears from the gastro-intestinal tract or a specified segment of the gastro-intestinal tract, e.g. the small intestine. Nutrient digestibility may be measured as the difference between what is administered to the subject and what. comes out in the faeces of the subject, or between what is administered to the subject and what remains in the digesta on a specified segment of the gastro intestinal tract, e.g. the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed. Nutrient digestibility as used herein encompasses starch digestibility, fat digestibility, protein digestibility, and amino acid digestibility.

Energy digestibility as used herein means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

Operably linked: The term “operably linked” means a configuration in which a control sequence is placed at an appropriate position relative to the coding sequence of a polynucleotide such that the control sequence directs expression of the coding sequence.

Percentage solubilised xylan: The term “percentage solubilised xylan” means the amount of xylose measured in the supernatant after incubation with an enzyme compared to the total amount of xylose present in the substrate before the incubation with the enzyme. For the purpose of the present invention, the percentage solubilised xylan may be calculated using defatted destarched maize (DFDSM) as substrate. DFDSM is prepared according to ‘Preparation of Defatted Destarched Maize (DFDSM)’ in the experimental section.

The percentage solubilised xylan from defatted destarched maize (DFDSM) may be determined using the reaction conditions 20 μg enzyme/g DFDSM and incubation at 40° C., pH 5 for 2.5 hours as described in the ‘Xylose solubilization assay’ herein. Thus the term ‘is performed under the reaction conditions 20 μg GH30 polypeptide per gram defatted destarched maize (DFDSM) and incubation at 40° C., pH 5 for 2.5 hours’ is to be understood that the percentage solubilised xylan is calculated as described in the ‘Xylose solubilization assay’ herein.

In a more detailed embodiment, 2% (w/w) DFDSM suspension was prepared in 100 mM sodium acetate, 5mM CaCl₂, pH 5 and allowed to hydrate for 30 min at room temperature under gently stirring. After hydration, 200 μl substrate suspension was pipetted into a 96 well plate and mixed with 20 μl enzyme solution to obtain a final enzyme concentration of 20 PPM relative to substrate (20 μg enzyme/g substrate). The enzyme/substrate mixtures were left for hydrolysis in 2.5 h at 40 ° C. under gently agitation (500 RPM) in a plate incubator. After enzymatic hydrolysis, the enzyme/substrate plates were centrifuged for 10 min at 3000 RPM and 50 μl supernatant was mixed with 100 μl 1.6 M HCl and transferred to 300 μl PCR tubes and left for acid hydrolysis for 40 min at 90 ° C. in a PCR machine. Samples were neutralized with 125 μl 1.4 M NaOH after acid hydrolysis and loaded on the HPAE-PAD for mono-saccharide analysis.

Sequence Identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Version 6.1.0 was used. The optional parameters used are gap open penalty of 10, gap extension penalty of 0.5, and the EBLOSUM62 (EMBOSS version of BLOSUM62) substitution matrix. The output of Needle labelled “longest identity” (obtained using the—nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. Version 6.1.0 was used. The optional parameters used are gap open penalty of 10, gap extension penalty of 0.5, and the EDNAFULL (EMBOSS version of NCBl NUC4.4) substitution matrix. The output of Needle labelled “longest identity” (obtained using the—nobrief option) is used as the percent identity and is calculated as follows:

(Identical Deoxyribonucleotides×100)/(Length of Alignment−Total Number of Gaps in Alignment)

Stringency conditions: The different stringency conditions are defined as follows.

The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2.0×SSC, 0.2% SDS at 60° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 1.0×SSC, 0.2% SDS at 60° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 1.0×SSC, 0.2% SDS at 65° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 1.0×SSC, 0.2% SDS at 70° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.5×SSC, 0.2% SDS at 70° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.5×SSC, 0.2% SDS at 75° C.

Subsequence: The term “subsequence” means a polynucleotide having one or more (e.g., several) nucleotides absent from the 5′ and/or 3′ end of a mature polypeptide coding sequence; wherein the subsequence encodes a fragment having xylanase activity.

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well-known recombinant methods or by classical purification methods.

Variant: The term “variant” means a polypeptide having xylanase activity comprising an alteration, i.e., a substitution, insertion, and/or deletion of one or more (several) amino acid residues at one or more (several) positions. A substitution means a replacement of an amino acid occupying a position with a different amino acid; a deletion means removal of an amino acid occupying a position; and an insertion means adding 1-3 amino acids adjacent to an amino acid occupying a position.

Xylanase: The term “xylanase” means a 1,4-beta-D-xylan-xylohydrolase (E.C. 3.2.1.8) that catalyses the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

### Nomenclature

For purposes of the present invention, the nomenclature [Y/F] means that the amino acid at this position may be a tyrosine (Try, Y) or a phenylalanine (Phe, F). Likewise the nomenclature [V/G/A/I] means that the amino acid at this position may be a valine (Val, V), glycine (Gly, G), alanine (Ala, A) or isoleucine (Ile, I), and so forth for other combinations as described herein. The amino acid X is defined such that it may be any of the 20 natural amino acids, unless otherwise stated.

### DETAILED DESCRIPTION OF THE INVENTION

The inventors have found that certain xylanases from glycoside hydrolase family 30 subfamily 8 (herein referred to as GH30_8) are surprisingly good at degrading the xylose backbone of sterically hindered arabinoxylan found in plant based material from the sub-family Panicoideae, thereby solubilising increased amounts of arabinoxylan which is measured as arabinose and xylose. Increased degradation, and thereby increased arabinose and xylose release, can result in advantages for many industries which use plant based material from the sub-family Panicoideae. This is surprising since xylanases from other glycoside hydrolase families (such as GH10 or GH11 either alone or in combination with GH43 or GH51 arabinofuranosidases) that are known to solubilise wheat are unable to solubilise the backbone of sterically hindered arabinoxylan found in plant based material from the sub-family Panicoideae.

The amount of starch present in untreated plant material makes it difficult to detect significant solubilisation of arabinoxylan. Thus model substrates, wherein the starch and fat present in the plant material is removed without effecting the degree of substitution, can be used to aid the determination of improved enzyme combinations over known prior art combinations. One model substrate is defatted destarched maize (DFDSM) and can be prepared as described in the experimental section herein. It is important that the model substrate is not prepared using strongly acidic or basic conditions or high temperatures, since such conditions can remove the side chain carbohydrate molecules and/or ester groups present on the xylan backbone. If these side chain groups are removed, then the complexity and degree of substitution will be reduced resulting in an arabinoxylan material which is easy to degrade by known solutions. It is for this reason that heat, acid and/or base pre-treatment is used in biomass conversion.

In order to measure the solubilisation of the arabinoxylan, the soluble arabinoxylan is hydrolysed with acid resulting in xylose and arabinose being released into the supernatant. This xylose and arabinose is then detected using e.g. the HPLC method as described herein. Thus the higher the degree of solubilisation of the arabinoxylan, the higher the amount of xylose and arabinose will be released upon acid hydrolysis. Without wishing to be bound by theory, it is believed that increasing the solubilisation of the arabinoxylan opens up the cell walls that can result in the nutrients, such as starch and protein, which are trapped inside being released. The release of starch and other nutrients can result in improved animal performance and/or improve the nutritional value of an animal feed.

The xylanases that have this surprising property all comprise the motif YXWWY[I/L]RRXYG (SEQ ID NO: 1). As far as the inventors are aware, this motif is only found in xylanases from GH30 subfamily 8. Such motifs are present in the polypeptides of the invention.

### Methods of Solubilising Xylan

In a first aspect, the invention relates to method of solubilising xylan from plant based material, comprising treating plant based material with a GH30 subfamily 8 polypeptide having xylanase activity, wherein the GH30 subfamily 8 polypeptide having xylanase activity is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In one embodiment, the polypeptide comprises the motif YXWWY[I/L]RRXYG (SEQ ID NO: 1).

In an embodiment, the polypeptide comprises or consists of amino acids 1 to 557 of SEQ

ID NO: 60. In an embodiment, the polypeptide comprises or consists of amino acids 1 to 569 of SEQ ID NO: 63. In an embodiment, the polypeptide comprises or consists of amino acids 1 to 382 of SEQ ID NO: 6. In an embodiment, the polypeptide comprises or consists of amino acids 1 to 390 of SEQ ID NO: 9. In an embodiment, the polypeptide comprises or consists of amino acids 1 to 391 of SEQ ID NO: 12. In an embodiment, the polypeptide comprises or consists of amino acids 1 to 399 of SEQ ID NO: 15. In an embodiment, the polypeptide comprises or consists of amino acids 1 to 383 of SEQ ID NO: 18. In an embodiment, the polypeptide comprises or consists of amino acids 1 to 391 of SEQ ID NO: 21. In an embodiment, the polypeptide comprises or consists of amino acids 1 to 565 of SEQ ID NO: 24. In an embodiment, the polypeptide comprises or consists of amino acids 1 to 573 of SEQ ID NO: 27. In an embodiment, the polypeptide comprises or consists of amino acids 1 to 396 of SEQ ID NO: 30. In an embodiment, the polypeptide comprises or consists of amino acids 1 to 404 of SEQ ID NO: 33. In an embodiment, the polypeptide comprises or consists of amino acids 1 to 392 of SEQ ID NO: 36. In an embodiment, the polypeptide comprises or consists of amino acids 1 to 400 of SEQ ID NO: 39. In an embodiment, the polypeptide comprises or consists of amino acids 1 to 413 of SEQ ID NO: 42. In an embodiment, the polypeptide comprises or consists of amino acids 1 to 421 of SEQ ID NO: 45. In an embodiment, the polypeptide comprises or consists of amino acids 1 to 398 of SEQ ID NO: 48. In an embodiment, the polypeptide comprises or consists of amino acids 1 to 406 of SEQ ID NO: 51. In an embodiment, the polypeptide comprises or consists of amino acids 1 to 382 of SEQ ID NO: 54. In an embodiment, the polypeptide comprises or consists of amino acids 1 to 390 of SEQ ID NO: 57.

In one embodiment, the percentage solubilised xylan is at least 4% when the method is performed under the reaction conditions 20 μg GH30 polypeptide per gram defatted destarched maize (DFDSM) and incubation at 40° C., pH 5 for 2.5 hours. In one embodiment, the percentage solubilised xylan is at least 4%, such as at least 4.5%, at least 5%, at least 5.5%, at least 6%, at least 6.5%, at least 7%, at least 7.5%, at least 8%, at least 8.5% or at least 9.0%.

In a preferred embodiment, the plant based material is from the sub-family Panicoideae. In a further embodiment, the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof. In a further embodiment, the plant based material from the sub-family Panicoideae is from the seed fraction (such as endosperm and/or husk) of the plant.

In one embodiment, the invention relates to method of solubilising xylan from plant based material, comprising treating plant based material with a GH30 subfamily 8 polypeptide having xylanase activity, wherein the GH30 subfamily 8 polypeptide having xylanase activity is selected from the group consisting of:
(a) a polypeptide having at least 85% sequence identity to the polypeptide of SEQ ID NO: 60;
(b) a polypeptide having at least 85% sequence identity to the polypeptide of SEQ ID NO: 6;
(c) a polypeptide having at least 92% sequence identity to the polypeptide of SEQ ID NO: 12;
(d) a polypeptide having at least 85% sequence identity to the polypeptide of SEQ ID NO: 18;
(e) a polypeptide having at least 85% sequence identity to the polypeptide of SEQ ID NO: 24;
(f) a polypeptide having at least 85% sequence identity to the polypeptide of SEQ ID NO: 30;
(g) a polypeptide having at least 85% sequence identity to the polypeptide of SEQ ID NO: 36;
(h) a polypeptide having at least 85% sequence identity to the polypeptide of SEQ ID NO: 42;
(i) a polypeptide having at least 80% sequence identity to the polypeptide of SEQ ID NO: 48;
(j) a polypeptide having at least 80% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide;

and wherein percentage solubilised xylan is at least 4% when the method is performed under the reaction conditions 20 μg GH30 polypeptide per gram defatted destarched maize (DFDSM) and incubation at 40° C., pH 5 for 2.5 hours. In one embodiment, the percentage solubilised xylan is at least 4.5%, such as at least 5%, at least 5.5%, at least 6%, at least 6.5%, at least 7%, at least 7.5%, at least 8%, at least 8.5% or at least 9.0%. In one embodiment, the polypeptide comprises the motif YXWWY[I/L]RRXYG (SEQ ID NO: 1).

In one embodiment, the invention relates to method of solubilising xylan from plant based material, comprising treating plant based material with a GH30 subfamily 8 polypeptide having xylanase activity, wherein the GH30 subfamily 8 polypeptide having xylanase activity is selected from the group consisting of:
(a) a polypeptide having at least 90% sequence identity to the polypeptide of SEQ ID NO: 60;
(b) a polypeptide having at least 90% sequence identity to the polypeptide of SEQ ID NO: 6;
(c) a polypeptide having at least 92% sequence identity to the polypeptide of SEQ ID NO: 12;
(d) a polypeptide having at least 90% sequence identity to the polypeptide of SEQ ID NO: 18;
(e) a polypeptide having at least 90% sequence identity to the polypeptide of SEQ ID NO: 24;
(f) a polypeptide having at least 90% sequence identity to the polypeptide of SEQ ID NO: 30;
(g) a polypeptide having at least 90% sequence identity to the polypeptide of SEQ ID NO: 36;
(h) a polypeptide having at least 90% sequence identity to the polypeptide of SEQ ID NO: 42;
(i) a polypeptide having at least 90% sequence identity to the polypeptide of SEQ ID NO: 48;
(j) a polypeptide having at least 90% sequence identity to the polypeptide of SEQ ID NO: 54;
(k) a variant of the polypeptide of SEQ ID NO: 60, SEQ ID NO: 6, SEQ ID NO: 12, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 positions;
(l) a polypeptide comprising the polypeptide of (a), (b), (c), (d), (e), (f), (g), (h), (i), (j) or (k) and a N-terminal and/or C-terminal His-tag and/or HQ-tag; and
(m) a fragment of a polypeptide of (a), (b), (c), (d), (e), (f), (g), (h), (i), (j), (k) or (l) having at least 90% of the length of the mature polypeptide;

and wherein percentage solubilised xylan is at least 5% when the method is performed under the reaction conditions 20 μg GH30 polypeptide per gram defatted destarched maize (DFDSM) and incubation at 40° C., pH 5 for 2.5 hours. In one embodiment, the percentage solubilised xylan is at least 5.5%, such as at least 6%, at least 6.5%, at least 7%, at least 7.5%, at least 8%, at least 8.5% or at least 9.0%. In one embodiment, the polypeptide comprises the motif YXWWY[I/L]RRXYG (SEQ ID NO: 1).

In one embodiment, the invention relates to method of solubilising xylan from plant based material, comprising treating plant based material with a GH30 subfamily 8 polypeptide having xylanase activity, wherein the GH30 subfamily 8 polypeptide having xylanase activity is selected from the group consisting of:
(a) a polypeptide having at least 90% sequence identity to the polypeptide of SEQ ID NO: 60;
(b) a polypeptide having at least 90% sequence identity to the polypeptide of SEQ ID NO: 6;
(c) a polypeptide having at least 92% sequence identity to the polypeptide of SEQ ID NO: 12;
(d) a polypeptide having at least 90% sequence identity to the polypeptide of SEQ ID NO: 24;
(e) a polypeptide having at least 90% sequence identity to the polypeptide of SEQ ID NO: 36;
(f) a polypeptide having at least 90% sequence identity to the polypeptide of SEQ ID NO: 42;
(g) a polypeptide having at least 90% sequence identity to the polypeptide of SEQ ID NO: 48;
(h) a polypeptide having at least 90% sequence identity to the polypeptide of SEQ ID NO: 54;
(i) a variant of the polypeptide of SEQ ID NO: 60, SEQ ID NO: 6, SEQ ID NO: 12, SEQ ID NO: 24, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 positions;
(j) a polypeptide comprising the polypeptide of (a), (b), (c), (d), (e), (f), (g), (h) or (i) and a N-terminal and/or C-terminal His-tag and/or HQ-tag; and
(k) a fragment of a polypeptide of (a), (b), (c), (d), (e), (f), (g), (h), (i) or (j) having at least 90% of the length of the mature polypeptide;

and wherein percentage solubilised xylan is at least 8% when the method is performed under the reaction conditions 20 μg GH30 polypeptide per gram defatted destarched maize (DFDSM) and incubation at 40° C., pH 5 for 2.5 hours. In one embodiment, the polypeptide comprises the motif YXWWY[I/L]RRXYG (SEQ ID NO: 1).

In a further embodiment, the invention relates to method of solubilising xylan from plant based material, comprising treating plant based material with a GH30 polypeptide having xylanase activity, wherein the GH30 polypeptide having xylanase activity has a sequence identity to the mature polypeptide of SEQ ID NO: 60 of at least 80%. In an embodiment, the polypeptide has a sequence identity to SEQ ID NO: 60 of at least 85%. In an embodiment, the polypeptide has a sequence identity to SEQ ID NO: 60 of at least 86%. In an embodiment, the polypeptide has a sequence identity to SEQ ID NO: 60 of at least 87%. In an embodiment, the polypeptide has a sequence identity to SEQ ID NO: 60 of at least 88%. In an embodiment, the polypeptide has a sequence identity to SEQ ID NO: 60 of at least 89%. In an embodiment, the polypeptide has a sequence identity to SEQ ID NO: 60 of at least 90%. In an embodiment, the polypeptide has a sequence identity to SEQ ID NO: 60 of at least 91%. In an embodiment, the polypeptide has a sequence identity to SEQ ID NO: 60 of at least 92%. In an embodiment, the polypeptide has a sequence identity to SEQ ID NO: 60 of at least 93%. In an embodiment, the polypeptide has a sequence identity to SEQ ID NO: 60 of at least 94%. In an embodiment, the polypeptide has a sequence identity to SEQ ID NO: 60 of at least 95%. In an embodiment, the polypeptide has a sequence identity to SEQ ID NO: 60 of at least 96%. In an embodiment, the polypeptide has a sequence identity to SEQ ID NO: 60 of at least 97%. In an embodiment, the polypeptide has a sequence identity to SEQ ID NO: 60 of at least 98%. In an embodiment, the polypeptide has a sequence identity to SEQ ID NO: 60 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 60. In an embodiment, the polypeptide has at least 60%, at least 70%, at least 75%, at least 80%, at least 85%, at least 90%, at least 95%, or at least 100% of the activity of the polypeptide of SEQ ID NO: 60.

In one embodiment, the polypeptide preferably comprises or consists of the amino acid sequence of SEQ ID NO: 59 and/or SEQ ID NO: 60 or an allelic variant thereof; comprises the amino acid sequence of SEQ ID NO: 59 and/or SEQ ID NO: 60 and a N-terminal and/or C-terminal His-tag and/or HQ-tag; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 59. In another embodiment, the polypeptide comprises or consists of the mature polypeptide of SEQ ID NO: 62. In another embodiment, the polypeptide comprises or consists of amino acids 1 to 557 of SEQ ID NO: 59. In another embodiment, the polypeptide comprises or consists of amino acids 1 to 557 of SEQ ID NO: 60. In another embodiment, the polypeptide comprises or consists of amino acids 1 to 569 of SEQ ID NO: 62. In another embodiment, the polypeptide comprises or consists of amino acids 1 to 569 of SEQ ID NO: 63. In an embodiment, the polypeptide has been isolated.

In an embodiment, the invention relates to met4%hod of solubilising xylan from plant based material, comprising treating plant based material with a GH30 polypeptide having xylanase activity, wherein the GH30 polypeptide having xylanase activity is a variant of SEQ ID NO: 60 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 60 is not more than 10, e.g., 1, 2, 3, 4, 5, 6, 7, 8, 9 or 10. In another embodiment, the number of substitutions, deletions, and/or insertions in SEQ ID NO: 60 is not more than 10, e.g., 1, 2, 3, 4, 5, 6, 7, 8, 9 or 10. In a further embodiment, the number of substitutions, preferably conservative substitutions, in SEQ ID NO: 60 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 60.

In one embodiment, the polypeptide comprises the motif YXWWY[I/L]RRXYG (SEQ ID NO: 1).

In a further embodiment, the invention relates to method of solubilising xylan from plant based material, comprising treating plant based material with a GH30 polypeptide having xylanase activity, wherein the GH30 polypeptide having xylanase activity has a sequence identity to the mature polypeptide of SEQ ID NO: 18 of at least 80%. In an embodiment, the polypeptide has a sequence identity to SEQ ID NO: 18 of at least 85%. In an embodiment, the polypeptide has a sequence identity to SEQ ID NO: 18 of at least 86%. In an embodiment, the polypeptide has a sequence identity to SEQ ID NO: 18 of at least 87%. In an embodiment, the polypeptide has a sequence identity to SEQ ID NO: 18 of at least 88%. In an embodiment, the polypeptide has a sequence identity to SEQ ID NO: 18 of at least 89%. In an embodiment, the polypeptide has a sequence identity to SEQ ID NO: 18 of at least 90%. In an embodiment, the polypeptide has a sequence identity to SEQ ID NO: 18 of at least 91%. In an embodiment, the polypeptide has a sequence identity to SEQ ID NO: 18 of at least 92%. In an embodiment, the polypeptide has a sequence identity to SEQ ID NO: 18 of at least 93%. In an embodiment, the polypeptide has a sequence identity to SEQ ID NO: 18 of at least 94%. In an embodiment, the polypeptide has a sequence identity to SEQ ID NO: 18 of at least 95%. In an embodiment, the polypeptide has a sequence identity to SEQ ID NO: 18 of at least 96%. In an embodiment, the polypeptide has a sequence identity to SEQ ID NO: 18 of at least 97%. In an embodiment, the polypeptide has a sequence identity to SEQ ID NO: 18 of at least 98%. In an embodiment, the polypeptide has a sequence identity to SEQ ID NO: 18 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 18. In an embodiment, the polypeptide has at least 60%, at least 70%, at least 75%, at least 80%, at least 85%, at least 90%, at least 95%, or at least 100% of the activity of the polypeptide of SEQ ID NO: 18.

In one embodiment, the polypeptide preferably comprises or consists of the amino acid sequence of SEQ ID NO: 17 and/or SEQ ID NO: 18 or an allelic variant thereof; comprises the amino acid sequence of SEQ ID NO: 17 and/or SEQ ID NO: 18 and a N-terminal and/or C-terminal His-tag and/or HQ-tag such as SEQ ID NO: 21; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 17. In another embodiment, the polypeptide comprises or consists of the mature polypeptide of SEQ ID NO: 20. In another embodiment, the polypeptide comprises or consists of amino acids 1 to 383 of SEQ ID NO: 17. In another embodiment, the polypeptide comprises or consists of amino acids 1 to 383 of SEQ ID NO: 18. In another embodiment, the polypeptide comprises or consists of amino acids 1 to 391 of SEQ ID NO: 20. In another embodiment, the polypeptide comprises or consists of amino acids 1 to 391 of SEQ ID NO: 21. In an embodiment, the polypeptide has been isolated.

In an embodiment, the invention relates to method of solubilising xylan from plant based material, comprising treating plant based material with a GH30 polypeptide having xylanase activity, wherein the GH30 polypeptide having xylanase activity is a variant of SEQ ID NO: 18 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 18 is not more than 10, e.g., 1, 2, 3, 4, 5, 6, 7, 8, 9 or 10. In another embodiment, the number of substitutions, deletions, and/or insertions in SEQ ID NO: 18 is not more than 10, e.g., 1, 2, 3, 4, 5, 6, 7, 8, 9 or 10. In a further embodiment, the number of substitutions, preferably conservative substitutions, in SEQ ID NO: 18 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 18.

In one embodiment, the polypeptide comprises the motif YXWWY[I/L]RRXYG (SEQ ID NO: 1).

In a further embodiment, the invention relates to method of solubilising xylan from plant based material, comprising treating plant based material with a GH30 polypeptide having xylanase activity, wherein the GH30 polypeptide having xylanase activity has a sequence identity to the mature polypeptide of SEQ ID NO: 24 of at least 80%. In an embodiment, the polypeptide has a sequence identity to SEQ ID NO: 24 of at least 85%. In an embodiment, the polypeptide has a sequence identity to SEQ ID NO: 24 of at least 86%. In an embodiment, the polypeptide has a sequence identity to SEQ ID NO: 24 of at least 87%. In an embodiment, the polypeptide has a sequence identity to SEQ ID NO: 24 of at least 88%. In an embodiment, the polypeptide has a sequence identity to SEQ ID NO: 24 of at least 89%. In an embodiment, the polypeptide has a sequence identity to SEQ ID NO: 24 of at least 90%. In an embodiment, the polypeptide has a sequence identity to SEQ ID NO: 24 of at least 91%. In an embodiment, the polypeptide has a sequence identity to SEQ ID NO: 24 of at least 92%. In an embodiment, the polypeptide has a sequence identity to SEQ ID NO: 24 of at least 93%. In an embodiment, the polypeptide has a sequence identity to SEQ ID NO: 24 of at least 94%. In an embodiment, the polypeptide has a sequence identity to SEQ ID NO: 24 of at least 95%. In an embodiment, the polypeptide has a sequence identity to SEQ ID NO: 24 of at least 96%. In an embodiment, the polypeptide has a sequence identity to SEQ ID NO: 24 of at least 97%. In an embodiment, the polypeptide has a sequence identity to SEQ ID NO: 24 of at least 98%. In an embodiment, the polypeptide has a sequence identity to SEQ ID NO: 24 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 24. In an embodiment, the polypeptide has at least 60%, at least 70%, at least 75%, at least 80%, at least 85%, at least 90%, at least 95%, or at least 100% of the activity of the polypeptide of SEQ ID NO: 24.

In one embodiment, the polypeptide preferably comprises or consists of the amino acid sequence of SEQ ID NO: 23 and/or SEQ ID NO: 24 or an allelic variant thereof; comprises the amino acid sequence of SEQ ID NO: 23 and/or SEQ ID NO: 24 and a N-terminal and/or C-terminal His-tag and/or HQ-tag such as SEQ ID NO: 27; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 23. In another embodiment, the polypeptide comprises or consists of the mature polypeptide of SEQ ID NO: 26. In another embodiment, the polypeptide comprises or consists of amino acids 1 to 565 of SEQ ID NO: 23. In another embodiment, the polypeptide comprises or consists of amino acids 1 to 565 of SEQ ID NO: 24. In another embodiment, the polypeptide comprises or consists of amino acids 1 to 573 of SEQ ID NO: 26. In another embodiment, the polypeptide comprises or consists of amino acids 1 to 573 of SEQ ID NO: 27. In an embodiment, the polypeptide has been isolated.

In an embodiment, the invention relates to method of solubilising xylan from plant based material, comprising treating plant based material with a GH30 polypeptide having xylanase activity, wherein the GH30 polypeptide having xylanase activity is a variant of SEQ ID NO: 24 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 24 is not more than 10, e.g., 1, 2, 3, 4, 5, 6, 7, 8, 9 or 10. In another embodiment, the number of substitutions, deletions, and/or insertions in SEQ ID NO: 24 is not more than 10, e.g., 1, 2, 3, 4, 5, 6, 7, 8, 9 or 10. In a further embodiment, the number of substitutions, preferably conservative substitutions, in SEQ ID NO: 24 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 24.

In one embodiment, the polypeptide comprises the motif YXWWY[I/L]RRXYG (SEQ ID NO: 1).

In a further embodiment, the invention relates to method of solubilising xylan from plant based material, comprising treating plant based material with a GH30 polypeptide having xylanase activity, wherein the GH30 polypeptide having xylanase activity has a sequence identity to the mature polypeptide of SEQ ID NO: 36 of at least 80%. In an embodiment, the polypeptide has a sequence identity to SEQ ID NO: 36 of at least 85%. In an embodiment, the polypeptide has a sequence identity to SEQ ID NO: 36 of at least 86%. In an embodiment, the polypeptide has a sequence identity to SEQ ID NO: 36 of at least 87%. In an embodiment, the polypeptide has a sequence identity to SEQ ID NO: 36 of at least 88%. In an embodiment, the polypeptide has a sequence identity to SEQ ID NO: 36 of at least 89%. In an embodiment, the polypeptide has a sequence identity to SEQ ID NO: 36 of at least 90%. In an embodiment, the polypeptide has a sequence identity to SEQ ID NO: 36 of at least 91%. In an embodiment, the polypeptide has a sequence identity to SEQ ID NO: 36 of at least 92%. In an embodiment, the polypeptide has a sequence identity to SEQ ID NO: 36 of at least 93%. In an embodiment, the polypeptide has a sequence identity to SEQ ID NO: 36 of at least 94%. In an embodiment, the polypeptide has a sequence identity to SEQ ID NO: 36 of at least 95%. In an embodiment, the polypeptide has a sequence identity to SEQ ID NO: 36 of at least 96%. In an embodiment, the polypeptide has a sequence identity to SEQ ID NO: 36 of at least 97%. In an embodiment, the polypeptide has a sequence identity to SEQ ID NO: 36 of at least 98%. In an embodiment, the polypeptide has a sequence identity to SEQ ID NO: 36 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 36. In an embodiment, the polypeptide has at least 60%, at least 70%, at least 75%, at least 80%, at least 85%, at least 90%, at least 95%, or at least 100% of the activity of the polypeptide of SEQ ID NO: 36.

In one embodiment, the polypeptide preferably comprises or consists of the amino acid sequence of SEQ ID NO: 35 and/or SEQ ID NO: 36 or an allelic variant thereof; comprises the amino acid sequence of SEQ ID NO: 35 and/or SEQ ID NO: 36 and a N-terminal and/or C-terminal His-tag and/or HQ-tag such as SEQ ID NO: 39; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 35. In another embodiment, the polypeptide comprises or consists of the mature polypeptide of SEQ ID NO: 38. In another embodiment, the polypeptide comprises or consists of amino acids 1 to 392 of SEQ ID NO: 35. In another embodiment, the polypeptide comprises or consists of amino acids 1 to 392 of SEQ ID NO: 36. In another embodiment, the polypeptide comprises or consists of amino acids 1 to 400 of SEQ ID NO: 38. In another embodiment, the polypeptide comprises or consists of amino acids 1 to 400 of SEQ ID NO: 39. In an embodiment, the polypeptide has been isolated.

In an embodiment, the invention relates to method of solubilising xylan from plant based material, comprising treating plant based material with a GH30 polypeptide having xylanase activity, wherein the GH30 polypeptide having xylanase activity is a variant of SEQ ID NO: 36 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 36 is not more than 10, e.g., 1, 2, 3, 4, 5, 6, 7, 8, 9 or 10. In another embodiment, the number of substitutions, deletions, and/or insertions in SEQ ID NO: 36 is not more than 10, e.g., 1, 2, 3, 4, 5, 6, 7, 8, 9 or 10. In a further embodiment, the number of substitutions, preferably conservative substitutions, in SEQ ID NO: 36 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 36.

In one embodiment, the polypeptide comprises the motif YXWWY[I/L]RRXYG (SEQ ID NO: 1).

### Polypeptides Having Xylanase Activity

In a second aspect, the invention relates to polypeptides having xylanase activity and having a sequence identity to the mature polypeptide of SEQ ID NO: 5 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 5.

In a continuation of the second aspect, the invention further relates to polypeptides having a sequence identity to SEQ ID NO: 6 of at least 80% which have xylanase activity. In an embodiment, the polypeptide has a sequence identity to SEQ ID NO: 6 of at least 85%. In an embodiment, the polypeptide has a sequence identity to SEQ ID NO: 6 of at least 86%. In an embodiment, the polypeptide has a sequence identity to SEQ ID NO: 6 of at least 87%. In an embodiment, the polypeptide has a sequence identity to SEQ ID NO: 6 of at least 88%. In an embodiment, the polypeptide has a sequence identity to SEQ ID NO: 6 of at least 89%. In an embodiment, the polypeptide has a sequence identity to SEQ ID NO: 6 of at least 90%. In an embodiment, the polypeptide has a sequence identity to SEQ ID NO: 6 of at least 91%. In an embodiment, the polypeptide has a sequence identity to SEQ ID NO: 6 of at least 92%. In an embodiment, the polypeptide has a sequence identity to SEQ ID NO: 6 of at least 93%. In an embodiment, the polypeptide has a sequence identity to SEQ ID NO: 6 of at least 94%. In an embodiment, the polypeptide has a sequence identity to SEQ ID NO: 6 of at least 95%. In an embodiment, the polypeptide has a sequence identity to SEQ ID NO: 6 of at least 96%. In an embodiment, the polypeptide has a sequence identity to SEQ ID NO: 6 of at least 97%. In an embodiment, the polypeptide has a sequence identity to SEQ ID NO: 6 of at least 98%. In an embodiment, the polypeptide has a sequence identity to SEQ ID NO: 6 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 6. In an embodiment, the polypeptide has at least 60%, at least 70%, at least 75%, at least 80%, at least 85%, at least 90%, at least 95%, or at least 100% of the activity of the polypeptide of SEQ ID NO: 6.

In a continuation of the second aspect, the invention relates to polypeptides having xylanase activity and having a sequence identity to the mature polypeptide of SEQ ID NO: 8 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. The invention further relates to polypeptides having xylanase activity and having a sequence identity to SEQ ID NO: 9 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 9.

In one embodiment, the polypeptide preferably comprises or consists of the amino acid sequence of SEQ ID NO: 5 and/or SEQ ID NO: 6 or an allelic variant thereof; comprises the amino acid sequence of SEQ ID NO: 5 and/or SEQ ID NO: 6 and a N-terminal and/or C-terminal His-tag and/or HQ-tag such as SEQ ID NO: 9; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 5.

In another embodiment, the polypeptide comprises or consists of the mature polypeptide of SEQ ID NO: 8. In another embodiment, the polypeptide comprises or consists of amino acids 1 to 382 of SEQ ID NO: 5. In another embodiment, the polypeptide comprises or consists of amino acids 1 to 382 of SEQ ID NO: 6. In another embodiment, the polypeptide comprises or consists of amino acids 1 to 390 of SEQ ID NO: 8. In another embodiment, the polypeptide comprises or consists of amino acids 1 to 390 of SEQ ID NO: 9. In an embodiment, the polypeptide has been isolated.

In a continuation of the second aspect, the invention relates to a polypeptide having xylanase activity encoded by a polynucleotide that hybridizes under high stringency conditions or very high stringency conditions with (i) the mature polypeptide coding sequence of SEQ ID NO: 4 or (ii) the full-length complement of (i) (Sambrook et al., 1989, Molecular Cloning, A Laboratory Manual, 2d edition, Cold Spring Harbor, N.Y.).

In a continuation of the second aspect, the invention relates to a polypeptide having xylanase activity encoded by a polynucleotide having a sequence identity to the mature polypeptide coding sequence of SEQ ID NO: 4 or the cDNA sequence thereof of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In a continuation of the second aspect, the invention relates to variants of SEQ ID NO: 6 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 6 is not more than 10, e.g., 1, 2, 3, 4, 5, 6, 7, 8, 9 or 10. In another embodiment, the number of substitutions, deletions, and/or insertions in SEQ ID NO: 6 is not more than 10, e.g., 1, 2, 3, 4, 5, 6, 7, 8, 9 or 10. In a further embodiment, the number of substitutions, preferably conservative substitutions, in SEQ ID NO: 6 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 6.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R.L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, LeuNal, Ala/Glu, and Asp/Gly. Other examples of conservative substitutions are G to A; A to G, S; V to I, L, A, T, S; I to V, L, M; L to I, M, V; M to L, I, V; P to A, S, N; F to Y, W, H; Y to F, W, H; W to Y, F, H; R to K, E, D; K to R, E, D; H to Q, N, S; D to N, E, K, R, Q; E to Q, D, K, R, N; S to T, A; T to S, V, A; C to S, T, A; N to D, Q, H, S; Q to E, N, H, K, R.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for arabinofuranosidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The catalytic amino acids of the GH30 xylanases were determined by alignment with a GH30 from Bacillus subtilis sp. 168. The first catalytic residue corresponding to E139 in the mature sequence of the GH30 xylanase from Bacillus subtilis (Uniprot Q45070) is E135, E139, E135, E136, E146, E139, E139, E142, E135 and E138 of SEQ ID NO: 6, 12, 18, 24, 30, 36, 42, 48, 54 and 60 respectfully. The second catalytic residue corresponding to E229 in the mature sequence of the GH30 xylanase from Bacillus subtilis (Uniprot Q45070) is E224, E228, E223, E235, E235, E228, E228, E231, E224, E227 and W229 of SEQ ID NO: 6, 12, 18, 24, 30, 36, 42, 48, 54 and 60 respectfully. Substitution or mutation in one of these positions is expected to deactivate the enzymatic activity.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Carbohydrate molecules are often attached to a polypeptide from a fungal source during post-translational modification. In order to aid mass spectrometry analysis, the polypeptide can be incubated with an endoglycosidase to deglycosylate each N-linked position. For every deglycosylated N-linked site, one N-acetyl hexosamine remains on the protein backbone.

In a third aspect, the invention relates to polypeptides having xylanase activity and having a sequence identity to the mature polypeptide of SEQ ID NO: 11 of at least 92%, e.g. at least 93%, at least 94%, at least 95%, at least 96%, at least 97%, at least 98%, at least 99%, which have xylanase activity. In one embodiment, the polypeptides differ by up to 39 amino acids, e.g., between 1 and 39 amino acids, such as 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 amino acids from the mature polypeptide of SEQ ID NO: 11.

In a continuation of the third aspect, the invention further relates to polypeptides having a sequence identity to SEQ ID NO: 12 of at least 92% which have xylanase activity. In an embodiment, the polypeptide has a sequence identity to SEQ ID NO: 12 of at least 93%. In an embodiment, the polypeptide has a sequence identity to SEQ ID NO: 12 of at least 94%. In an embodiment, the polypeptide has a sequence identity to SEQ ID NO: 12 of at least 95%. In an embodiment, the polypeptide has a sequence identity to SEQ ID NO: 12 of at least 96%. In an embodiment, the polypeptide has a sequence identity to SEQ ID NO: 12 of at least 97%. In an embodiment, the polypeptide has a sequence identity to SEQ ID NO: 12 of at least 98%. In an embodiment, the polypeptide has a sequence identity to SEQ ID NO: 12 of at least 99%.

In one embodiment, the polypeptides differ by up to 39 amino acids, e.g., between 1 and 39 amino acids, such as 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 amino acids from SEQ ID NO: 12. In an embodiment, the polypeptide has at least 60%, at least 70%, at least 75%, at least 80%, at least 85%, at least 90%, at least 95%, or at least 100% of the activity of the polypeptide of SEQ ID NO: 12.

In a continuation of the third aspect, the invention relates to polypeptides having xylanase activity and having a sequence identity to the mature polypeptide of SEQ ID NO: 14 of at least 92%, e.g., at least 93%, at least 94%, at least 95%, at least 96%, at least 97%, at least 98%, at least 99%, which have xylanase activity. The invention further relates to polypeptides having xylanase activity and having a sequence identity to SEQ ID NO: 15 of at least 92%, e.g., at least 93%, at least 94%, at least 95%, at least 96%, at least 97%, at least 98%, at least 99%, which have xylanase activity. In one embodiment, the polypeptides differ by up to 39 amino acids, e.g., between 1 and 39 amino acids, such as 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 amino acids from SEQ ID NO: 15.

In one embodiment, the polypeptide preferably comprises or consists of the amino acid sequence of SEQ ID NO: 11 and/or SEQ ID NO: 12 or an allelic variant thereof; comprises the amino acid sequence of SEQ ID NO: 11 and/or SEQ ID NO: 12 and a N-terminal and/or C-terminal

His-tag and/or HQ-tag such as SEQ ID NO: 15; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 11. In another embodiment, the polypeptide comprises or consists of the mature polypeptide of SEQ ID NO: 14. In another embodiment, the polypeptide comprises or consists of amino acids 1 to 391 of SEQ ID NO: 11. In another embodiment, the polypeptide comprises or consists of amino acids 1 to 391 of SEQ ID NO: 12. In another embodiment, the polypeptide comprises or consists of amino acids 1 to 399 of SEQ ID NO: 14. In another embodiment, the polypeptide comprises or consists of amino acids 1 to 399 of SEQ ID NO: 15. In an embodiment, the polypeptide has been isolated.

In a continuation of the third aspect, the invention relates to a polypeptide having xylanase activity encoded by a polynucleotide that hybridizes under very high stringency conditions with (i) the mature polypeptide coding sequence of SEQ ID NO: 10 or (ii) the full-length complement of (i).

In a continuation of the third aspect, the invention relates to a polypeptide having xylanase activity encoded by a polynucleotide having a sequence identity to the mature polypeptide coding sequence of SEQ ID NO: 10 or the cDNA sequence thereof of at least 92%, e.g., at least 93%, at least 94%, at least 95%, at least 96%, at least 97%, at least 98%, at least 99%, or 100%.

In a continuation of the third aspect, the invention relates to variants of SEQ ID NO: 12 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions. In an embodiment, the number of positions comprising one or more amino acid substitutions, and/or one or more amino acid deletions, and/or one or more amino acid insertions or any combination thereof in SEQ ID NO: 12 is not more than 10, e.g., 1, 2, 3, 4, 5, 6, 7, 8, 9 or 10. In another embodiment, the number of substitutions, deletions, and/or insertions in SEQ ID NO: 12 is not more than 10, e.g., 1, 2, 3, 4, 5, 6, 7, 8, 9 or 10. In a further embodiment, the number of substitutions, preferably conservative substitutions, in SEQ ID NO: 12 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 12. Examples of amino acid changes and conservative substitutions are described in the second aspect of the invention.

In a fourth aspect, the invention relates to polypeptides having xylanase activity and having a sequence identity to the mature polypeptide of SEQ ID NO: 29 of at least 86%, e.g., at at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 29.

In a continuation of the fourth aspect, the invention further relates to polypeptides having a sequence identity to SEQ ID NO: 30 of at least 86% which have xylanase activity. In an embodiment, the polypeptide has a sequence identity to SEQ ID NO: 30 of at least 87%. In an embodiment, the polypeptide has a sequence identity to SEQ ID NO: 30 of at least 88%. In an embodiment, the polypeptide has a sequence identity to SEQ ID NO: 30 of at least 89%. In an embodiment, the polypeptide has a sequence identity to SEQ ID NO: 30 of at least 90%. In an embodiment, the polypeptide has a sequence identity to SEQ ID NO: 30 of at least 91%. In an embodiment, the polypeptide has a sequence identity to SEQ ID NO: 30 of at least 92%. In an embodiment, the polypeptide has a sequence identity to SEQ ID NO: 30 of at least 93%. In an embodiment, the polypeptide has a sequence identity to SEQ ID NO: 30 of at least 94%. In an embodiment, the polypeptide has a sequence identity to SEQ ID NO: 30 of at least 95%. In an embodiment, the polypeptide has a sequence identity to SEQ ID NO: 30 of at least 96%. In an embodiment, the polypeptide has a sequence identity to SEQ ID NO: 30 of at least 97%. In an embodiment, the polypeptide has a sequence identity to SEQ ID NO: 30 of at least 98%. In an embodiment, the polypeptide has a sequence identity to SEQ ID NO: 30 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 30. In an embodiment, the polypeptide has at least 60%, at least 70%, at least 75%, at least 80%, at least 85%, at least 90%, at least 95%, or at least 100% of the activity of the polypeptide of SEQ ID NO: 30.

In a continuation of the fourth aspect, the invention relates to polypeptides having xylanase activity and having a sequence identity to the mature polypeptide of SEQ ID NO: 32 of at least 86%, e.g., at least 87%, at least 88%, at least 89%, at least 90%, at least 91%, at least 92%, at least 93%, at least 94%, at least 95%, at least 96%, at least 97%, at least 98%, at least 99%, which have xylanase activity. The invention further relates to polypeptides having xylanase activity and having a sequence identity to SEQ ID NO: 33 of at least 86%, e.g., at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 33.

In one embodiment, the polypeptide preferably comprises or consists of the amino acid sequence of SEQ ID NO: 29 and/or SEQ ID NO: 30 or an allelic variant thereof; comprises the amino acid sequence of SEQ ID NO: 29 and/or SEQ ID NO: 30 and a N-terminal and/or C-terminal His-tag and/or HQ-tag such as SEQ ID NO: 33; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 29. In another embodiment, the polypeptide comprises or consists of the mature polypeptide of SEQ ID NO: 32. In another embodiment, the polypeptide comprises or consists of amino acids 1 to 396 of SEQ ID NO: 29. In another embodiment, the polypeptide comprises or consists of amino acids 1 to 396 of SEQ ID NO: 30. In another embodiment, the polypeptide comprises or consists of amino acids 1 to 404 of SEQ ID NO: 32. In another embodiment, the polypeptide comprises or consists of amino acids 1 to 404 of SEQ ID NO: 33. In an embodiment, the polypeptide has been isolated.

In a continuation of the fourth aspect, the invention relates to a polypeptide having xylanase activity encoded by a polynucleotide that hybridizes under high stringency conditions or very high stringency conditions with (i) the mature polypeptide coding sequence of SEQ ID NO: 28 or (ii) the full-length complement of (i).

In a continuation of the fourth aspect, the invention relates to a polypeptide having xylanase activity encoded by a polynucleotide having a sequence identity to the mature polypeptide coding sequence of SEQ ID NO: 28 or the cDNA sequence thereof of at least 86%, e.g., at least 87%, at least 88%, at least 89%, at least 90%, at least 91%, at least 92%, at least 93%, at least 94%, at least 95%, at least 96%, at least 97%, at least 98%, at least 99%, or 100%.

In a continuation of the fourth aspect, the invention relates to variants of SEQ ID NO: 30 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 30 is not more than 10, e.g., 1, 2, 3, 4, 5, 6, 7, 8, 9 or 10. In another embodiment, the number of substitutions, deletions, and/or insertions in SEQ ID NO: 30 is not more than 10, e.g., 1, 2, 3, 4, 5, 6, 7, 8, 9 or 10. In a further embodiment, the number of substitutions, preferably conservative substitutions, in SEQ ID NO: 30 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 30. Examples of amino acid changes and conservative substitutions are described in the second aspect of the invention.

In a fifth aspect, the invention relates to polypeptides having xylanase activity and having a sequence identity to the mature polypeptide of SEQ ID NO: 41 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 41.

In a continuation of the fifth aspect, the invention further relates to polypeptides having a sequence identity to SEQ ID NO: 42 of at least 85% which have xylanase activity. In an embodiment, the polypeptide has a sequence identity to SEQ ID NO: 42 of at least 86%. In an embodiment, the polypeptide has a sequence identity to SEQ ID NO: 42 of at least 87%. In an embodiment, the polypeptide has a sequence identity to SEQ ID NO: 42 of at least 88%. In an embodiment, the polypeptide has a sequence identity to SEQ ID NO: 42 of at least 89%. In an embodiment, the polypeptide has a sequence identity to SEQ ID NO: 42 of at least 90%. In an embodiment, the polypeptide has a sequence identity to SEQ ID NO: 42 of at least 91%. In an embodiment, the polypeptide has a sequence identity to SEQ ID NO: 42 of at least 92%. In an embodiment, the polypeptide has a sequence identity to SEQ ID NO: 42 of at least 93%. In an embodiment, the polypeptide has a sequence identity to SEQ ID NO: 42 of at least 94%. In an embodiment, the polypeptide has a sequence identity to SEQ ID NO: 42 of at least 95%. In an embodiment, the polypeptide has a sequence identity to SEQ ID NO: 42 of at least 96%. In an embodiment, the polypeptide has a sequence identity to SEQ ID NO: 42 of at least 97%. In an embodiment, the polypeptide has a sequence identity to SEQ ID NO: 42 of at least 98%. In an embodiment, the polypeptide has a sequence identity to SEQ ID NO: 42 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 42. In an embodiment, the polypeptide has at least 60%, at least 70%, at least 75%, at least 80%, at least 85%, at least 90%, at least 95%, or at least 100% of the activity of the polypeptide of SEQ ID NO: 42.

In a continuation of the fifth aspect, the invention relates to polypeptides having xylanase activity and having a sequence identity to the mature polypeptide of SEQ ID NO: 44 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. The invention further relates to polypeptides having xylanase activity and having a sequence identity to SEQ ID NO: 45 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 45.

In one embodiment, the polypeptide preferably comprises or consists of the amino acid sequence of SEQ ID NO: 41 and/or SEQ ID NO: 42 or an allelic variant thereof; comprises the amino acid sequence of SEQ ID NO: 41 and/or SEQ ID NO: 42 and a N-terminal and/or C-terminal His-tag and/or HQ-tag such as SEQ ID NO: 45; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 41. In another embodiment, the polypeptide comprises or consists of the mature polypeptide of SEQ ID NO: 44. In another embodiment, the polypeptide comprises or consists of amino acids 1 to 413 of SEQ ID NO: 41. In another embodiment, the polypeptide comprises or consists of amino acids 1 to 413 of SEQ ID NO: 42. In another embodiment, the polypeptide comprises or consists of amino acids 1 to 421 of SEQ ID NO: 44. In another embodiment, the polypeptide comprises or consists of amino acids 1 to 421 of SEQ ID NO: 45. In an embodiment, the polypeptide has been isolated.

In a continuation of the fifth aspect, the invention relates to a polypeptide having xylanase activity encoded by a polynucleotide that hybridizes under high stringency conditions or very high stringency conditions with (i) the mature polypeptide coding sequence of SEQ ID NO: 40 or (ii) the full-length complement of (i).

In a continuation of the fifth aspect, the invention relates to a polypeptide having xylanase activity encoded by a polynucleotide having a sequence identity to the mature polypeptide coding sequence of SEQ ID NO: 40 or the cDNA sequence thereof of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In a continuation of the fifth aspect, the invention relates to variants of SEQ ID NO: 42 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 42 is not more than 10, e.g., 1, 2, 3, 4, 5, 6, 7, 8, 9 or 10. In another embodiment, the number of substitutions, deletions, and/or insertions in SEQ ID NO: 42 is not more than 10, e.g., 1, 2, 3, 4, 5, 6, 7, 8, 9 or 10. In a further embodiment, the number of substitutions, preferably conservative substitutions, in SEQ ID NO: 42 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 42. Examples of amino acid changes and conservative substitutions are described in the second aspect of the invention.

In a sixth aspect, the invention relates to polypeptides having xylanase activity and having a sequence identity to the mature polypeptide of SEQ ID NO: 47 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 47.

In a continuation of the sixth aspect, the invention further relates to polypeptides having a sequence identity to SEQ ID NO: 48 of at least 80% which have xylanase activity. In an embodiment, the polypeptide has a sequence identity to SEQ ID NO: 48 of at least 85%. In an embodiment, the polypeptide has a sequence identity to SEQ ID NO: 48 of at least 86%. In an embodiment, the polypeptide has a sequence identity to SEQ ID NO: 48 of at least 87%. In an embodiment, the polypeptide has a sequence identity to SEQ ID NO: 48 of at least 88%. In an embodiment, the polypeptide has a sequence identity to SEQ ID NO: 48 of at least 89%. In an embodiment, the polypeptide has a sequence identity to SEQ ID NO: 48 of at least 90%. In an embodiment, the polypeptide has a sequence identity to SEQ ID NO: 48 of at least 91%. In an embodiment, the polypeptide has a sequence identity to SEQ ID NO: 48 of at least 92%. In an embodiment, the polypeptide has a sequence identity to SEQ ID NO: 48 of at least 93%. In an embodiment, the polypeptide has a sequence identity to SEQ ID NO: 48 of at least 94%. In an embodiment, the polypeptide has a sequence identity to SEQ ID NO: 48 of at least 95%. In an embodiment, the polypeptide has a sequence identity to SEQ ID NO: 48 of at least 96%. In an embodiment, the polypeptide has a sequence identity to SEQ ID NO: 48 of at least 97%. In an embodiment, the polypeptide has a sequence identity to SEQ ID NO: 48 of at least 98%. In an embodiment, the polypeptide has a sequence identity to SEQ ID NO: 48 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 48. In an embodiment, the polypeptide has at least 60%, at least 70%, at least 75%, at least 80%, at least 85%, at least 90%, at least 95%, or at least 100% of the activity of the polypeptide of SEQ ID NO: 48.

In a continuation of the sixth aspect, the invention relates to polypeptides having xylanase activity and having a sequence identity to the mature polypeptide of SEQ ID NO: 50 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. The invention further relates to polypeptides having xylanase activity and having a sequence identity to SEQ ID NO: 51 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 51.

In one embodiment, the polypeptide preferably comprises or consists of the amino acid sequence of SEQ ID NO: 47 and/or SEQ ID NO: 48 or an allelic variant thereof; comprises the amino acid sequence of SEQ ID NO: 47 and/or SEQ ID NO: 48 and a N-terminal and/or C-terminal His-tag and/or HQ-tag such as SEQ ID NO: 51; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 47. In another embodiment, the polypeptide comprises or consists of the mature polypeptide of SEQ ID NO: 50. In another embodiment, the polypeptide comprises or consists of amino acids 1 to 398 of SEQ ID NO: 47. In another embodiment, the polypeptide comprises or consists of amino acids 1 to 398 of SEQ ID NO: 48. In another embodiment, the polypeptide comprises or consists of amino acids 1 to 406 of SEQ ID NO: 50. In another embodiment, the polypeptide comprises or consists of amino acids 1 to 406 of SEQ ID NO: 51. In an embodiment, the polypeptide has been isolated.

In a continuation of the sixth aspect, the invention relates to a polypeptide having xylanase activity encoded by a polynucleotide that hybridizes under high stringency conditions or very high stringency conditions with (i) the mature polypeptide coding sequence of SEQ ID NO: 52 or (ii) the full-length complement of (i).

In a continuation of the sixth aspect, the invention relates to a polypeptide having xylanase activity encoded by a polynucleotide having a sequence identity to the mature polypeptide coding sequence of SEQ ID NO: 52 or the cDNA sequence thereof of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In a continuation of the sixth aspect, the invention relates to variants of SEQ ID NO: 48 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 48 is not more than 10, e.g., 1, 2, 3, 4, 5, 6, 7, 8, 9 or 10. In another embodiment, the number of substitutions, deletions, and/or insertions in SEQ ID NO: 48 is not more than 10, e.g., 1, 2, 3, 4, 5, 6, 7, 8, 9 or 10. In a further embodiment, the number of substitutions, preferably conservative substitutions, in SEQ ID NO: 48 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 48. Examples of amino acid changes and conservative substitutions are described in the second aspect of the invention.

In a seventh aspect, the invention relates to polypeptides having xylanase activity and having a sequence identity to the mature polypeptide of SEQ ID NO: 53 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from the mature polypeptide of SEQ ID NO: 53.

In a continuation of the seventh aspect, the invention further relates to polypeptides having a sequence identity to SEQ ID NO: 54 of at least 80% which have xylanase activity. In an embodiment, the polypeptide has a sequence identity to SEQ ID NO: 54 of at least 85%. In an embodiment, the polypeptide has a sequence identity to SEQ ID NO: 54 of at least 86%. In an embodiment, the polypeptide has a sequence identity to SEQ ID NO: 54 of at least 87%. In an embodiment, the polypeptide has a sequence identity to SEQ ID NO: 54 of at least 88%. In an embodiment, the polypeptide has a sequence identity to SEQ ID NO: 54 of at least 89%. In an embodiment, the polypeptide has a sequence identity to SEQ ID NO: 54 of at least 90%. In an embodiment, the polypeptide has a sequence identity to SEQ ID NO: 54 of at least 91%. In an embodiment, the polypeptide has a sequence identity to SEQ ID NO: 54 of at least 92%. In an embodiment, the polypeptide has a sequence identity to SEQ ID NO: 54 of at least 93%. In an embodiment, the polypeptide has a sequence identity to SEQ ID NO: 54 of at least 94%. In an embodiment, the polypeptide has a sequence identity to SEQ ID NO: 54 of at least 95%. In an embodiment, the polypeptide has a sequence identity to SEQ ID NO: 54 of at least 96%. In an embodiment, the polypeptide has a sequence identity to SEQ ID NO: 54 of at least 97%. In an embodiment, the polypeptide has a sequence identity to SEQ ID NO: 54 of at least 98%. In an embodiment, the polypeptide has a sequence identity to SEQ ID NO: 54 of at least 99%.

In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 54. In an embodiment, the polypeptide has at least 60%, at least 70%, at least 75%, at least 80%, at least 85%, at least 90%, at least 95%, or at least 100% of the activity of the polypeptide of SEQ ID NO: 54.

In a continuation of the seventh aspect, the invention relates to polypeptides having xylanase activity and having a sequence identity to the mature polypeptide of SEQ ID NO: 56 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. The invention further relates to polypeptides having xylanase activity and having a sequence identity to SEQ ID NO: 57 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, which have xylanase activity. In one embodiment, the polypeptides differ by up to 50 amino acids, e.g., between 1 and 50 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids from SEQ ID NO: 57.

In one embodiment, the polypeptide preferably comprises or consists of the amino acid sequence of SEQ ID NO: 53 and/or SEQ ID NO: 54 or an allelic variant thereof; comprises the amino acid sequence of SEQ ID NO: 53 and/or SEQ ID NO: 54 and a N-terminal and/or C-terminal His-tag and/or HQ-tag such as SEQ ID NO: 57; or is a fragment thereof having xylanase activity and having at least 90% such as at least 91%, at least 92%, at least 93%, at least 94%, at least 95%, at least 96%, at least 97%, at least 98% or at least 99% of the length of the mature polypeptide. In another embodiment, the polypeptide comprises or consists of the mature polypeptide of SEQ ID NO: 53. In another embodiment, the polypeptide comprises or consists of the mature polypeptide of SEQ ID NO: 56. In another embodiment, the polypeptide comprises or consists of amino acids 1 to 382 of SEQ ID NO: 53. In another embodiment, the polypeptide comprises or consists of amino acids 1 to 382 of SEQ ID NO: 54. In another embodiment, the polypeptide comprises or consists of amino acids 1 to 390 of SEQ ID NO: 56. In another embodiment, the polypeptide comprises or consists of amino acids 1 to 390 of SEQ ID NO: 57. In an embodiment, the polypeptide has been isolated.

In a continuation of the seventh aspect, the invention relates to a polypeptide having xylanase activity encoded by a polynucleotide that hybridizes under high stringency conditions or very high stringency conditions with (i) the mature polypeptide coding sequence of SEQ ID NO: 52 or (ii) the full-length complement of (i).

In a continuation of the seventh aspect, the invention relates to a polypeptide having xylanase activity encoded by a polynucleotide having a sequence identity to the mature polypeptide coding sequence of SEQ ID NO: 52 or the cDNA sequence thereof of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In a continuation of the seventh aspect, the invention relates to variants of SEQ ID NO: 54 having xylanase activity comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In an embodiment, the number of positions comprising one or more amino acid substitutions, and/or one or more amino acid deletions, and/or one or more amino acid insertions or any combination thereof in SEQ ID NO: 54 is not more than 10, e.g., 1, 2, 3, 4, 5, 6, 7, 8, 9 or 10. In another embodiment, the number of substitutions, deletions, and/or insertions in SEQ ID NO: 54 is not more than 10, e.g., 1, 2, 3, 4, 5, 6, 7, 8, 9 or 10. In a further embodiment, the number of substitutions, preferably conservative substitutions, in SEQ ID NO: 54 is not more than 10, e.g., 1, 2, 3, 4, 5, 6, 7, 8, 9 or 10. In an embodiment, the variant has at least 60%, at least 70%, at least 75%, at least 80%, at least 85%, at least 90%, at least 95%, or at least 100% of the activity of the polypeptide of SEQ ID NO: 54. Examples of amino acid changes and conservative substitutions are described in the second aspect of the invention.

Polypeptides Solubilising Xylan from Plant Based Material from the Sub-Family Panicoideae

In an embodiment, the GH30 subfamily 8 polypeptide of the invention solubilises at least 4% solubilized xylan from plant based material from the sub-family Panicoideae when the method is performed under the reaction conditions 20 μg GH30 polypeptide per gram defatted destarched maize (DFDSM) and incubation at 40° C., pH 5 for 2.5 hours. In an embodiment, the GH30 polypeptide is the polypeptide of one or more, such as all, of aspects two, three, four, five, six and/or seven of the invention. In an embodiment, the percentage solubilised xylan is at least 4.5%, such as at least 5%, at least 5.5%, at least 6% or at least 6.5%.

In a preferred embodiment, the GH30 subfamily 8 polypeptide solubilises at least 7% solubilized xylan from plant based material from the sub-family Panicoideae when the method is performed under the reaction conditions 20 μg GH30 polypeptide per gram defatted destarched maize (DFDSM) and incubation at 40° C., pH 5 for 2.5 hours. In an embodiment, the GH30 polypeptide is the polypeptide of one or more, such as all, of aspects two, three, five, six and/or seven of the invention. In an embodiment, the percentage solubilised xylan is at least 7.5%, such as at least 8%, at least 8.5% or at least 9.0%.

In a more preferred embodiment, the GH30 subfamily 8 polypeptide solubilises at least 9.5% solubilized xylan from plant based material from the sub-family Panicoideae when the method is performed under the reaction conditions 20 μg GH30 polypeptide per gram defatted destarched maize (DFDSM) and incubation at 40° C., pH 5 for 2.5 hours. In an embodiment, the GH30 polypeptide is the polypeptide of one or more, such as all, of aspects two, three, five and/or seven of the invention.

In an embodiment, the GH30 subfamily 8 polypeptide of any of aspects two, three, four, five, six and/or seven comprises one or more motifs YXWWY[I/L]RRXYG (SEQ ID NO: 1). In a preferred embodiment, the GH30 subfamily 8 polypeptide of any of aspects two, three, five, six and/or seven comprises one or more motifs YXWWY[I/L]RRXYG (SEQ ID NO: 1) and solubilises at least 8% solubilized xylan from plant based material from the sub-family Panicoideae when the method is performed under the reaction conditions 20 μg GH30 polypeptide per gram defatted destarched maize (DFDSM) and incubation at 40° C., pH 5 for 2.5 hours.

Granules Comprising Polypeptides having Xylanase Activity

The invention further relates to a granule comprising one or more GH30 subfamily 8 polypeptide having xylanase activity, wherein the polypeptide is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In one embodiment, the polypeptide comprises the motif YXWWY[I/L]RRXYG (SEQ ID NO: 1).

In an embodiment, the polypeptide comprises or consists of amino acids 1 to 557 of SEQ ID NO: 60. In an embodiment, the polypeptide comprises or consists of amino acids 1 to 569 of SEQ ID NO: 63. In an embodiment, the polypeptide comprises or consists of amino acids 1 to 382 of SEQ ID NO: 6. In an embodiment, the polypeptide comprises or consists of amino acids 1 to 390 of SEQ ID NO: 9. In an embodiment, the polypeptide comprises or consists of amino acids 1 to 391 of SEQ ID NO: 12. In an embodiment, the polypeptide comprises or consists of amino acids 1 to 399 of SEQ ID NO: 15. In an embodiment, the polypeptide comprises or consists of amino acids 1 to 383 of SEQ ID NO: 18. In an embodiment, the polypeptide comprises or consists of amino acids 1 to 391 of SEQ ID NO: 21. In an embodiment, the polypeptide comprises or consists of amino acids 1 to 565 of SEQ ID NO: 24. In an embodiment, the polypeptide comprises or consists of amino acids 1 to 573 of SEQ ID NO: 27. In an embodiment, the polypeptide comprises or consists of amino acids 1 to 396 of SEQ ID NO: 30. In an embodiment, the polypeptide comprises or consists of amino acids 1 to 404 of SEQ ID NO: 33. In an embodiment, the polypeptide comprises or consists of amino acids 1 to 392 of SEQ ID NO: 36. In an embodiment, the polypeptide comprises or consists of amino acids 1 to 400 of SEQ ID NO: 39. In an embodiment, the polypeptide comprises or consists of amino acids 1 to 413 of SEQ ID NO: 42. In an embodiment, the polypeptide comprises or consists of amino acids 1 to 421 of SEQ ID NO: 45. In an embodiment, the polypeptide comprises or consists of amino acids 1 to 398 of SEQ ID NO: 48. In an embodiment, the polypeptide comprises or consists of amino acids 1 to 406 of SEQ ID NO: 51. In an embodiment, the polypeptide comprises or consists of amino acids 1 to 382 of SEQ ID NO: 54. In an embodiment, the polypeptide comprises or consists of amino acids 1 to 390 of SEQ ID NO: 57.

In one embodiment, the granule comprises one or more formulating agents (such as those described herein), preferably a formulating agent selected from the list consisting of glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose, preferably selected from the list consisting of 1,2-propylene glycol, 1,3-propylene glycol, sodium sulfate, dextrin, cellulose, sodium thiosulfate, kaolin and calcium carbonate.

In one embodiment, the granule comprises a core particle and one or more coatings. In an embodiment, the coating comprises salt and/or wax and/or flour.

In one embodiment, the granule comprises one or more additional enzymes. The one or more additional enzymes is preferably selected from the group consisting of phytase, xylanase, galactanase, alpha-galactosidase, beta-galactosidase, protease, phospholipase Al, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.

In one embodiment, the granule comprises one or more probiotics. The one or more probiotics is preferably selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof.

In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from defatted destarched maize (DFDSM). In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from DFDSM under the reaction conditions 20 μg GH30 polypeptide per gram DFDSM and incubation at 40° C., pH 5 for 2.5 hours.

### Methods of Improving Animal Performance

The invention further relates to a method of improving one or more performance parameters of an animal, comprising administering to one or more animals a GH30 subfamily 8 polypeptide having xylanase activity and plant based material from the sub-family Panicoideae, wherein the GH30 subfamily 8 polypeptide is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In one embodiment, the polypeptide comprises the motif YXWWY[I/L]RRXYG (SEQ ID NO: 1).

The plant based material from the sub-family Panicoideae may be administered together or separately with the GH30 subfamily 8 polypeptide. The GH30 subfamily 8 polypeptide may be administered e.g. in a composition or in an animal feed additive. In an embodiment, the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof. In a further embodiment, the the plant based material from the sub-family Panicoideae is from the seed fraction (such as endosperm and/or husk) of the plant.

In an embodiment, the polypeptide comprises or consists of amino acids 1 to 557 of SEQ ID NO: 60. In an embodiment, the polypeptide comprises or consists of amino acids 1 to 569 of SEQ ID NO: 63. In an embodiment, the polypeptide comprises or consists of amino acids 1 to 382 of SEQ ID NO: 6. In an embodiment, the polypeptide comprises or consists of amino acids 1 to 390 of SEQ ID NO: 9. In an embodiment, the polypeptide comprises or consists of amino acids 1 to 391 of SEQ ID NO: 12. In an embodiment, the polypeptide comprises or consists of amino acids 1 to 399 of SEQ ID NO: 15. In an embodiment, the polypeptide comprises or consists of amino acids 1 to 383 of SEQ ID NO: 18. In an embodiment, the polypeptide comprises or consists of amino acids 1 to 391 of SEQ ID NO: 21. In an embodiment, the polypeptide comprises or consists of amino acids 1 to 565 of SEQ ID NO: 24. In an embodiment, the polypeptide comprises or consists of amino acids 1 to 573 of SEQ ID NO: 27. In an embodiment, the polypeptide comprises or consists of amino acids 1 to 396 of SEQ ID NO: 30. In an embodiment, the polypeptide comprises or consists of amino acids 1 to 404 of SEQ ID NO: 33. In an embodiment, the polypeptide comprises or consists of amino acids 1 to 392 of SEQ ID NO: 36. In an embodiment, the polypeptide comprises or consists of amino acids 1 to 400 of SEQ ID NO: 39. In an embodiment, the polypeptide comprises or consists of amino acids 1 to 413 of SEQ ID NO: 42. In an embodiment, the polypeptide comprises or consists of amino acids 1 to 421 of SEQ ID NO: 45. In an embodiment, the polypeptide comprises or consists of amino acids 1 to 398 of SEQ ID NO: 48. In an embodiment, the polypeptide comprises or consists of amino acids 1 to 406 of SEQ ID NO: 51. In an embodiment, the polypeptide comprises or consists of amino acids 1 to 382 of SEQ ID NO: 54. In an embodiment, the polypeptide comprises or consists of amino acids 1 to 390 of SEQ ID NO: 57.

In one embodiment, ‘improving the performance of an animal’ means that there is an increase in body weight gain. In another embodiment, ‘improving the performance of an animal’ means that there is an improved feed conversion ratio. In a further embodiment, ‘improving the performance of an animal’ means that there is an increased feed efficiency. In a further embodiment, ‘improving the performance of an animal’ means that there is an increase in body weight gain and/or an improved feed conversion ratio and/or an increased feed efficiency.

In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from defatted destarched maize (DFDSM). In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from DFDSM under the reaction conditions 20 μg GH30 polypeptide per gram DFDSM and incubation at 40° C., pH 5 for 2.5 hours.

### Methods of Preparing an Animal Feed

The invention further relates to a method of preparing an animal feed, comprising mixing a GH30 subfamily 8 polypeptide having xylanase activity with plant based material from the sub-family Panicoideae, wherein the GH30 subfamily 8 polypeptide is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In one embodiment, the polypeptide comprises the motif YXWWY[I/L]RRXYG (SEQ ID NO. 1).

The GH30 subfamily 8 polypeptide may be e.g. in the form of a composition or an animal feed additive. In an embodiment, the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof. In a further embodiment, the the plant based material from the sub-family Panicoideae is from the seed fraction (such as endosperm and/or husk) of the plant.

In an embodiment, the polypeptide comprises or consists of amino acids 1 to 557 of SEQ ID NO: 60. In an embodiment, the polypeptide comprises or consists of amino acids 1 to 569 of SEQ ID NO: 63. In an embodiment, the polypeptide comprises or consists of amino acids 1 to 382 of SEQ ID NO: 6. In an embodiment, the polypeptide comprises or consists of amino acids 1 to 390 of SEQ ID NO: 9. In an embodiment, the polypeptide comprises or consists of amino acids 1 to 391 of SEQ ID NO: 12. In an embodiment, the polypeptide comprises or consists of amino acids 1 to 399 of SEQ ID NO: 15. In an embodiment, the polypeptide comprises or consists of amino acids 1 to 383 of SEQ ID NO: 18. In an embodiment, the polypeptide comprises or consists of amino acids 1 to 391 of SEQ ID NO: 21. In an embodiment, the polypeptide comprises or consists of amino acids 1 to 565 of SEQ ID NO: 24. In an embodiment, the polypeptide comprises or consists of amino acids 1 to 573 of SEQ ID NO: 27. In an embodiment, the polypeptide comprises or consists of amino acids 1 to 396 of SEQ ID NO: 30. In an embodiment, the polypeptide comprises or consists of amino acids 1 to 404 of SEQ ID NO: 33. In an embodiment, the polypeptide comprises or consists of amino acids 1 to 392 of SEQ ID NO: 36. In an embodiment, the polypeptide comprises or consists of amino acids 1 to 400 of SEQ ID NO: 39. In an embodiment, the polypeptide comprises or consists of amino acids 1 to 413 of SEQ ID NO: 42. In an embodiment, the polypeptide comprises or consists of amino acids 1 to 421 of SEQ ID NO: 45. In an embodiment, the polypeptide comprises or consists of amino acids 1 to 398 of SEQ ID NO: 48. In an embodiment, the polypeptide comprises or consists of amino acids 1 to 406 of SEQ ID NO: 51. In an embodiment, the polypeptide comprises or consists of amino acids 1 to 382 of SEQ ID NO: 54. In an embodiment, the polypeptide comprises or consists of amino acids 1 to 390 of SEQ ID NO: 57.

In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from defatted destarched maize (DFDSM). In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from DFDSM under the reaction conditions 20 μg GH30 polypeptide per gram DFDSM and incubation at 40° C., pH 5 for 2.5 hours.

### Method for Improving the Nutritional Value of Animal Feed

The term improving the nutritional value of an animal feed means improving the availability of nutrients in the feed. The nutritional values refers in particular to improving the solubilisation and degradation of the arabinoxylan-containing fraction (e.g. such as hemicellulose) of the feed, thereby leading to increased release of nutrients from cells in the endosperm that have cell walls composed of highly recalcitrant hemicellulose. Consequently, an increased release of arabinoxylan oligomers indicates a disruption of the cell walls and as a result the nutritional value of the feed is improved resulting in increased growth rate and/or weight gain and/or feed conversion (i.e. the weight of ingested feed relative to weight gain). In addition the arabinoxylan oligomer release may result in improved utilization of these components per se either directly or by bacterial fermentation in the hind gut thereby resulting in a production of short chain fatty acids that may be readily absorbed in the hind and utilised in the energy metabolism.

Thus the invention further relates to a method for improving the nutritional value of an animal feed comprising plant based material from the sub-family Panicoideae, comprising adding to the feed a GH30 subfamily 8 polypeptide having xylanase activity, wherein the GH30 subfamily 8 polypeptide is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In one embodiment, the polypeptide comprises the motif YXWWY[I/L]RRXYG (SEQ ID NO. 1).

The GH30 subfamily 8 polypeptide may be e.g. in the form of a composition or an animal feed additive. In an embodiment, the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof. In a further embodiment, the the plant based material from the sub-family Panicoideae is from the seed fraction (such as endosperm and/or husk) of the plant.

In an embodiment, the polypeptide comprises or consists of amino acids 1 to 557 of SEQ ID NO: 60. In an embodiment, the polypeptide comprises or consists of amino acids 1 to 569 of SEQ ID NO: 63. In an embodiment, the polypeptide comprises or consists of amino acids 1 to 382 of SEQ ID NO: 6. In an embodiment, the polypeptide comprises or consists of amino acids 1 to 390 of SEQ ID NO: 9. In an embodiment, the polypeptide comprises or consists of amino acids 1 to 391 of SEQ ID NO: 12. In an embodiment, the polypeptide comprises or consists of amino acids 1 to 399 of SEQ ID NO: 15. In an embodiment, the polypeptide comprises or consists of amino acids 1 to 383 of SEQ ID NO: 18. In an embodiment, the polypeptide comprises or consists of amino acids 1 to 391 of SEQ ID NO: 21. In an embodiment, the polypeptide comprises or consists of amino acids 1 to 565 of SEQ ID NO: 24. In an embodiment, the polypeptide comprises or consists of amino acids 1 to 573 of SEQ ID NO: 27. In an embodiment, the polypeptide comprises or consists of amino acids 1 to 396 of SEQ ID NO: 30. In an embodiment, the polypeptide comprises or consists of amino acids 1 to 404 of SEQ ID NO: 33. In an embodiment, the polypeptide comprises or consists of amino acids 1 to 392 of SEQ ID NO: 36. In an embodiment, the polypeptide comprises or consists of amino acids 1 to 400 of SEQ ID NO: 39. In an embodiment, the polypeptide comprises or consists of amino acids 1 to 413 of SEQ ID NO: 42. In an embodiment, the polypeptide comprises or consists of amino acids 1 to 421 of SEQ ID NO: 45. In an embodiment, the polypeptide comprises or consists of amino acids 1 to 398 of SEQ ID NO: 48. In an embodiment, the polypeptide comprises or consists of amino acids 1 to 406 of SEQ ID NO: 51. In an embodiment, the polypeptide comprises or consists of amino acids 1 to 382 of SEQ ID NO: 54. In an embodiment, the polypeptide comprises or consists of amino acids 1 to 390 of SEQ ID NO: 57.

In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from defatted destarched maize (DFDSM). In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from DFDSM under the reaction conditions 20 μg GH30 polypeptide per gram DFDSM and incubation at 40° C., pH 5 for 2.5 hours.

### Methods of Releasing Starch

The invention further relates to a method of releasing starch from plant based material, comprising treating plant based material from the sub-family Panicoideae with a GH30 subfamily 8 polypeptide having xylanase activity, wherein the GH30 subfamily 8 polypeptide is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In one embodiment, the polypeptide comprises the motif YXWWY[I/L]RRXYG (SEQ ID NO: 1).

The GH30 subfamily 8 polypeptide may be e.g. in the form of a composition or an animal feed additive. In an embodiment, the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof. In a further embodiment, the the plant based material from the sub-family Panicoideae is from the seed fraction (such as endosperm and/or husk) of the plant.

In an embodiment, the polypeptide comprises or consists of amino acids 1 to 557 of SEQ ID NO: 60. In an embodiment, the polypeptide comprises or consists of amino acids 1 to 569 of SEQ ID NO: 63. In an embodiment, the polypeptide comprises or consists of amino acids 1 to 382 of SEQ ID NO: 6. In an embodiment, the polypeptide comprises or consists of amino acids 1 to 390 of SEQ ID NO: 9. In an embodiment, the polypeptide comprises or consists of amino acids 1 to 391 of SEQ ID NO: 12. In an embodiment, the polypeptide comprises or consists of amino acids 1 to 399 of SEQ ID NO: 15. In an embodiment, the polypeptide comprises or consists of amino acids 1 to 383 of SEQ ID NO: 18. In an embodiment, the polypeptide comprises or consists of amino acids 1 to 391 of SEQ ID NO: 21. In an embodiment, the polypeptide comprises or consists of amino acids 1 to 565 of SEQ ID NO: 24. In an embodiment, the polypeptide comprises or consists of amino acids 1 to 573 of SEQ ID NO: 27. In an embodiment, the polypeptide comprises or consists of amino acids 1 to 396 of SEQ ID NO: 30. In an embodiment, the polypeptide comprises or consists of amino acids 1 to 404 of SEQ ID NO: 33. In an embodiment, the polypeptide comprises or consists of amino acids 1 to 392 of SEQ ID NO: 36. In an embodiment, the polypeptide comprises or consists of amino acids 1 to 400 of SEQ ID NO: 39. In an embodiment, the polypeptide comprises or consists of amino acids 1 to 413 of SEQ ID NO: 42. In an embodiment, the polypeptide comprises or consists of amino acids 1 to 421 of SEQ ID NO: 45. In an embodiment, the polypeptide comprises or consists of amino acids 1 to 398 of SEQ ID NO: 48. In an embodiment, the polypeptide comprises or consists of amino acids 1 to 406 of SEQ ID NO: 51. In an embodiment, the polypeptide comprises or consists of amino acids 1 to 382 of SEQ ID NO: 54. In an embodiment, the polypeptide comprises or consists of amino acids 1 to 390 of SEQ ID NO: 57.

In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from defatted destarched maize (DFDSM). In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from DFDSM under the reaction conditions 20 μg GH30 polypeptide per gram DFDSM and incubation at 40° C., pH 5 for 2.5 hours.

### Sources of Polypeptides Having Xylanase Activity

A polypeptide having xylanase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. For example, the polypeptide may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces polypeptide having xylanase activity. In one embodiment, the polypeptide is from a bacterium of the class Bacilli, such as from the order Bacillales, or from the family Paenibacillaceae, or from the genus Paenibacillus or from the species Paenibacillus sp-19179 or Paenibacillus panacisoli.

In another embodiment, the polypeptide is from a bacterium of the class Clostridia, such as from the order Clostridiales, or from the family Clostridiaceae, or from the genus Clostridium or from the species Clostridium saccharobutylicum.

In another embodiment, the polypeptide is from a bacterium of the class Clostridia, such as from the order Clostridiales, or from the family Ruminococcaceae, or from the genus Ruminococcus, or from the species Ruminococcus sp. CAG:330.

In another embodiment, the polypeptide is from a bacterium of the class Gammaproteobacteria, such as from the order Alteromonadales, or from the family Pseudoalteromonadaceae, or from the genus Pseudoalteromonas or from the species Pseudoalteromonas tetraodonis.

In another embodiment, the polypeptide is from a bacterium of the class Gammaproteobacteria, such as from the order Enterobacteriales, or from the family Enterobacteriaceae, or from the genus Pectobacterium or from the species Pectobacterium carotovorum.

In another embodiment, the polypeptide is from a bacterium of the class Actinobacteria, such as from the order Streptomycetales, or from the family Streptomycetaceae, or from the genus Streptomyces or from the species Streptomyces sp-62627.

In another embodiment, the polypeptide is from a bacterium of the class Gammaproteobacteria, such as from the order Vibrionales, or from the family Vibrionaceae, or from the genus Vibrio or from the species Vibrio rhizosphaerae.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding a polypeptide of the present invention.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Paenibacillus or Chryseobacterium, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis ctyllIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis ctyllIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the

Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980). The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa,

Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Pseudoalteromonas tetraodonis cell. In another aspect, the cell is a Paenibacillus sp-19179 cell. In another aspect, the cell is a Streptomyces sp-62627 cell. In another aspect, the cell is a Clostridium saccharobutylicum cell. In another aspect, the cell is a Paenibacillus panacisoli cell. In another aspect, the cell is a Vibrio rhizosphaerae cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Plants

The present invention also relates to isolated plants, e.g., a transgenic plant, plant part, or plant cell, comprising a polynucleotide of the present invention so as to express and produce a polypeptide or domain in recoverable quantities. The polypeptide or domain may be recovered from the plant or plant part. Alternatively, the plant or plant part containing the polypeptide or domain may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot).

Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems.

Plant cells and specific plant cell compartments, such as chloroplasts, apoplasts, mitochondria, vacuoles, peroxisomes and cytoplasm are also considered to be a plant part.

Also included within the scope of the present invention are the progeny of such plants, plant parts, and plant cells.

The transgenic plant or plant cell expressing the polypeptide or domain may be constructed in accordance with methods known in the art.

The present invention also relates to methods of producing a polypeptide or domain of the present invention comprising (a) cultivating a transgenic plant or a plant cell comprising a polynucleotide encoding the polypeptide or domain under conditions conducive for production of the polypeptide or domain; and (b) recovering the polypeptide or domain.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a polypeptide of the present invention. Preferably, the compositions are enriched in the polypeptide of the invention. The term “enriched” indicates that the xylanase activity of the composition has been increased, e.g., with an enrichment factor of at least 1.1, such as at least 1.2, at least 1.3, at least 1.4, at least 1.5, at least 2.0, at least 3.0, at least 4.0, at least 5.0, at least 10.

In an embodiment, the composition comprises the polypeptide of the invention and one or more formulating agents, as described below.

The compositions may further comprise multiple enzymatic activities, such as one or more (e.g., several) enzymes selected from the group consisting of phytase, xylanase, galactanase, alpha-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.

The compositions may further comprise one or more microbes. In an embodiment, the microbe is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof.

### Formulation

The enzyme of the invention may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) ora sugar or sugar derivative (such as e.g. dextrin, glucose, sucrose, and sorbitol). Thus in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

For a solid formulation, the formulation may be for example as a granule, spray dried powder or agglomerate (e.g. as disclosed in WO2000/70034). The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In one embodiment, the composition is a solid composition, such as a spray dried composition, comprising the lysozyme of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate, magnesium sulfate and calcium carbonate.

The present invention also relates to enzyme granules/particles comprising the lysozyme of the invention optionally combined with one or more additional enzymes. The granule is composed of a core, and optionally one or more coatings (outer layers) surrounding the core.

Typically the granule/particle size, measured as equivalent spherical diameter (volume based average particle size), of the granule is 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier. Preparation methods include known feed and granule formulation technologies, e.g.:

a) spray dried products, wherein a liquid enzyme-containing solution is atomized in a spray drying tower to form small droplets which during their way down the drying tower dry to form an enzyme-containing particulate material;

b) layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606;

c) absorbed core particles, wherein rather than coating the enzyme as a layer around the core, the enzyme is absorbed onto and/or into the surface of the core. Such a process is described in WO 97/39116.

d) extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme;

e) prilled products, wherein an enzyme-containing powder is suspended in molten wax and the suspension is sprayed, e.g., through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. No. 4,016,040 and U.S. Pat. No. 4,713,245 are documents relating to this technique;

f) mixer granulation products, wherein a liquid is added to a dry powder composition of, e.g., conventional granulating components, the enzyme being introduced either via the liquid or the powder or both. The liquid and the powder are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granulates consisting of enzyme as enzyme, fillers and binders etc. are mixed with cellulose fibres to reinforce the particles to give the so-called T-granulate. Reinforced particles, being more robust, release less enzymatic dust.

g) size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons);

h) fluid bed granulation, which involves suspending particulates in an air stream and spraying a liquid onto the fluidized particles via nozzles. Particles hit by spray droplets get wetted and become tacky. The tacky particles collide with other particles and adhere to them and form a granule;

i) the cores may be subjected to drying, such as in a fluid bed drier. Other known methods for drying granules in the feed or detergent industry can be used by the skilled person. The drying preferably takes place at a product temperature of from 25 to 90° C. For some enzymes it is important the cores comprising the enzyme contain a low amount of water before coating. If water sensitive enzymes are coated before excessive water is removed, it will be trapped within the core and it may affect the activity of the enzyme negatively. After drying, the cores preferably contain 0.1-10% w/w water.

The core may include additional materials such as fillers, fibre materials (cellulose or synthetic fibres), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

In one embodiment, the core comprises a material selected from the group consisting of salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). In one embodiment, the core comprises a clay mineral such as kaolinite or kaolin.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt and/or wax and/or flour coating, or other suitable coating materials.

The coating may be applied in an amount of at least 0.1% by weight of the core, e.g., at least 0.5%, 1% or 5%. The amount may be at most 100%, 70%, 50%, 40% or 30%.

The coating is preferably at least 0.1 μm thick, particularly at least 0.5 μm, at least 1 μm or at least 5 μm. In some embodiments the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit is encapsulated or enclosed with few or no uncoated areas. The layer or coating should in particular be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, sorbate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO1997/05245, WO1998/54980, WO1998/55599, WO2000/70034, WO2006/034710, WO2008/017661, WO2008/017659, WO2000/020569, WO2001/004279, WO1997/05245, WO2000/01793, WO2003/059086, WO2003/059087, WO2007/031483, WO2007/031485, WO2007/044968, WO2013/192043, WO2014/014647 and WO2015/197719 or polymer coating such as described in WO 2001/00042.

Specific examples of suitable salts are NaCl (CH20° C.=76%), Na2CO3 (CH20° C.=92%), NaNO3 (CH20° C.=73%), Na2HPO4 (CH20° C.=95%), Na3PO4 (CH25° C.=92%), NH4Cl (CH20° C.=79.5%), (NH4)2HPO4 (CH20° C.=93,0%), NH4H2PO4 (CH20° C.=93.1%), (NH4)2SO4 (CH20° C.=81.1%), KCl (CH20° C.=85%), K2HPO4 (CH20° C.=92%), KH2PO4 (CH20° C.=96.5%), KNO3 (CH20° C.=93.5%), Na2SO4 (CH20° C.=93%), K2SO4 (CH20° C.=98%), KHSO4 (CH20° C.=86%), MgSO4 (CH20° C.=90%), ZnSO4 (CH20° C.=90%) and sodium citrate (CH25° C.=86%). Other examples include NaH2PO4, (NH4)H2PO4, CuSO4, Mg(NO3)2, magnesium acetate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, sodium acetate, sodium benzoate, sodium citrate, sodium sulfate, zinc acetate, zinc benzoate, zinc carbonate, zinc chloride, zinc citrate and zinc sorbate.

The salt may be in anhydrous form, or it may be a hydrated salt, i.e. a crystalline salt hydrate with bound water(s) of crystallization, such as described in WO 99/32595. Specific examples include anhydrous sodium sulfate (Na2SO4), anhydrous magnesium sulfate (MgSO4), magnesium sulfate heptahydrate (MgSO4.7H2O), zinc sulfate heptahydrate (ZnSO4.7H2O), sodium phosphate dibasic heptahydrate (Na2HPO4.7H2O), magnesium nitrate hexahydrate (Mg(NO3)2(6H2O)), sodium citrate dihydrate and magnesium acetate tetrahydrate.

Preferably the salt is applied as a solution of the salt, e.g., using a fluid bed.

A wax coating may comprise at least 60% by weight of a wax, e.g., at least 65%, at least 70%, at least 75%, at least 80%, at least 85%, at least 90%, at least 95% or at least 99% by weight.

Specific examples of waxes are polyethylene glycols; polypropylenes; Carnauba wax; Candelilla wax; bees wax; hydrogenated plant oil or animal tallow such as polyethylene glycol (PEG), methyl hydroxy-propyl cellulose (MHPC), polyvinyl alcohol (PVA), hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil.

The granule may comprise a core comprising the lysozyme of the invention, one or more salt coatings and one or more wax coatings. Examples of enzyme granules with multiple coatings are shown in WO1993/07263, WO1997/23606 and WO2016/149636.

Non-dusting granulates may be produced, e.g., as disclosed in U.S. Pat. Nos. 4,106,991 and 4,661,452 and may optionally be coated by methods known in the art. The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238,216.

Thus, in a further aspect, the present invention provides a granule, which comprises:

(a) a core comprising a lysozyme according to the invention, and

(b) a coating consisting of one or more layer(s) surrounding the core.

In one embodiment, the coating comprises a salt coating as described herein. In one embodiment, the coating comprises a wax coating as described herein. In one embodiment, the coating comprises a salt coating followed by a wax coating as described herein.

### Formulating Agent

The enzyme of the invention may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

For a solid formulation, the formulation may be for example as a granule, spray dried powder or agglomerate. The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In an embodiment, the solid composition is in granulated form. The granule may have a matrix structure where the components are mixed homogeneously. However, the granule typically comprises a core particle and one or more coatings, which typically are salt and/or wax coatings. The core particle can either be a homogeneous blend of xylanase of the invention optionally combined with one or more additional enzymes and optionally together with one or more salts or an inert particle with the xylanase of the invention optionally combined with one or more additional enzymes applied onto it.

In an embodiment, the material of the core particles are selected from the group consisting of inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals.

The salt coating is typically at least 1 μm thick and can either be one particular salt or a mixture of salts, such as Na₂SO₄, K₂SO₄, MgSO₄ and/or sodium citrate. Other examples are those described in e.g. WO 2008/017659, WO 2006/034710, WO 1997/05245, WO 1998/54980, WO 1998/55599, WO 2000/70034 or polymer coating such as described in WO 2001/00042.

In another embodiment, the composition is a solid composition comprising the xylanase of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate and calcium carbonate. In a preferred embodiment, the solid composition is in granulated form. In an embodiment, the solid composition is in granulated form and comprises a core particle, an enzyme layer comprising the xylanase of the invention and a salt coating.

In a further embodiment, the formulating agent is selected from one or more of the following compounds: glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: 1,2-propylene glycol, 1,3-propylene glycol, sodium sulfate, dextrin, cellulose, sodium thiosulfate and calcium carbonate.

Plant Based Material from the Sub-Family Panicoideae

In one embodiment, the plant based material from the sub-family Panicoideae is from the tribe Andropogoneae such as the rank Andropogon or Andropterum or Apluda or Apocopis or Arthraxon or Bothriochloa or Capillipedium or Chionachne or Chrysopogon or Coelorachis or Coix or Cymbopogon or Dichanthium or Diheteropogon or Dimeria or Elionurus or Eremochloa or Euclasta or Eulalia or Germainia or Hemarthria or Heteropholis or Heteropogon or Hyparrhenia or Hyperthelia or Imperata or Ischaemum or Iseilema or Kerriochloa or Microstegium or Miscanthidium or Miscanthus or Mnesithea or Ophiuros or Oxyrhachis or Phacelurus or Pholiurus or Pogonatherum or Polytoca or Polytrias or Pseudopogonatherum or Pseudosorghum or Rhytachne or Rottboellia or Saccharum or Sarga or Schizachyrium or Sehima or Sorghastrum or Sorghum or Spodiopogon or Thaumastochloa or Thelepogon or Themeda or Trachypogon or Triarrhena or Tripsacum or Urelytrum or Vetiveria or Vossia or Xerochloa or Zea.

In a preferred embodiment, the plant based material from the sub-family Panicoideae is from the rank Zea, such as the species Zea diploperennis, Zea luxurians, Zea mays, Zea nicaraguensis or Zea perennis.

In a preferred embodiment, the plant based material from the sub-family Panicoideae is from the rank Sorghum, such as the species Sorghum amplum, Sorghum angustum, Sorghum arundinaceum, Sorghum australiense, Sorghum bicolor, Sorghum brachypodum, Sorghum bulbosum, Sorghum ecarinatum, Sorghum exstans, Sorghum grande, Sorghum halepense, Sorghum hybrid cultivar, Sorghum interjectum, Sorghum intrans, Sorghum laxiflorum, Sorghum leiocladum, Sorghum macrospermum, Sorghum matarankense, Sorghum nitidum, Sorghum plumosum, Sorghum propinquum, Sorghum purpureosericeum, Sorghum stipoideum, Sorghum sudanense, Sorghum timorense, Sorghum versicolor, Sorghum sp. ‘Silk’ or Sorghum sp. as defined in WO2007/002267.

In another embodiment, the plant based material from the sub-family Panicoideae is from the tribe Paniceae such as the rank Acritochaete, Acroceras, Alexfloydia, Alloteropsis, Amphicarpum, Ancistrachne, Anthephora, Brachiaria, Calyptochloa, Cenchrus, Chaetium, Chaetopoa, Chamaeraphis, Chlorocalymma, Cleistochloa, Cyphochlaena, Cyrtococcum, Dichanthelium, Digitaria, Dissochondrus, Echinochloa, Entolasia, Eriochloa, Homopholis, Hygrochloa, Hylebates, Ixophorus, Lasiacis, Leucophrys, Louisiana, Megaloprotachne, Megathyrsus, Melinis, Microcalamus, Moorochloa, Neurachne, Odontelytrum, Oplismenus, Ottochloa, Panicum, Paractaenum, Paraneurachne, Paratheria, Parodiophyllochloa, Paspalidium, Pennisetum, Plagiosetum, Poecilostachys, Pseudechinolaena, Pseudochaetochloa, Pseudoraphis, Rupichloa, Sacciolepis, Scutachne, Setaria, Setariopsis, Snowdenia, Spinifex, Stenotaphrum, Stereochlaena, Thrasya, Thuarea, Thyridolepis, Tricholaena, unclassified Paniceae, Uranthoecium, Urochloa, Walwhalleya, Whiteochloa, Yakirra, Yvesia, Zuloagaea or Zygochloa.

In a preferred embodiment, the plant based material from the sub-family Panicoideae is from the rank Panicum, such as the species Panicum adenophorum, Panicum aff. aquaticum JKT-2012, Panicum amarum, Panicum antidotale, Panicum aquaticum, Panicum arctum, Panicum arundinariae, Panicum atrosanguineum, Panicum auricomum, Panicum auritum, Panicum bartlettii, Panicum bergii, Panicum bisulcatum, Panicum boliviense, Panicum brazzavillense, Panicum brevifolium, Panicum caaguazuense, Panicum campestre, Panicum capillare, Panicum cayennense, Panicum cayoense, Panicum cervicatum, Panicum chloroleucum, Panicum claytonii, Panicum coloratum, Panicum cyanescens, Panicum decompositum, Panicum deustum, Panicum dichotomiflorum, Panicum dinklagei, Panicum distichophyllum, Panicum dregeanum, Panicum elephantipes, Panicum fauriei, Panicum flexile, Panicum fluviicola, Panicum gouinii, Panicum gracilicaule, Panicum granuliferum, Panicum guatemalense, Panicum hallii, Panicum heterostachyum, Panicum hirticaule, Panicum hirtum, Panicum hylaeicum, Panicum incumbens, Panicum infestum, Panicum italicum, Panicum laetum, Panicum laevinode, Panicum lanipes, Panicum larcomianum, Panicum longipedicellatum, Panicum machrisianum, Panicum malacotrichum, Panicum margaritiferum, Panicum micranthum, Panicum miliaceum, Panicum milioides, Panicum millegrana, Panicum mystasipum, Panicum natalense, Panicum nephelophilum, Panicum nervosum, Panicum notatum, Panicum olyroides, Panicum paludosum, Panicum pansum, Panicum pantrichum, Panicum parvifolium, Panicum parviglume, Panicum pedersenii, Panicum penicillatum, Panicum petersonii, Panicum phragmitoides, Panicum piauiense, Panicum pilosum, Panicum pleianthurn, Panicum polycomum, Panicum polygonaturn, Panicum pseudisachne, Panicum pygmaeum, Panicum pyrularium, Panicum queenslandicum, Panicum racemosum, Panicum repens, Panicum rhizogonum, Panicum rigidulum, Panicum rivale, Panicum rude, Panicum rudgei, Panicum schinzii, Panicum schwackeanum, Panicum sellowii, Panicum seminudum, Panicum stapfianum, Panicum stenodes, Panicum stramineum, Panicum subalbidum, Panicum subtiramulosum, Panicum sumatrense, Panicum tenellum, Panicum tenuifolium, Panicum trichanthurn, Panicum trichidiachne, Panicum trichoides, Panicum tricholaenoides, Panicum tuerckheimii, Panicum turgidum, Panicum urvilleanurn, Panicum validum, Panicum venezuelae, Panicum verrucosum, Panicum virgatum, Panicum wettsteinii, Panicum sp., Panicum sp. Christin 16-200, Panicum sp. ELS-2011, Panicum sp. EM389 or Panicum sp. Forest 761.

In a further embodiment, the plant based material from the sub-family Panicoideae is maize (Zea), corn (Zea), sorghum (Sorghum), switchgrass (Panicum virgatum), millet (Panicum miliaceum), pearl millet (Cenchrus violaceus also called Pennisetum glaucum), foxtail millet (Setaria italica also called Panicum italicum) or in a processed form such as milled corn, milled maize, defatted maize, defatted destarched maize, milled sorghum, milled switchgrass, milled millet, milled foxtail millet, milled pearl millet, or any combination thereof.

In an embodiment, the plant based material from the sub-family Panicoideae is from the seed of the plant. In a preferred embodiment, the plant based material from the sub-family Panicoideae is from the seed of maize (Zea), corn (Zea), sorghum (Sorghum), switchgrass (Panicum virgatum), millet (Panicum miliaceum), pearl millet (Cenchrus violaceus also called Pennisetum glaucum), foxtail millet (Setaria italica also called Panicum italicum) or wherein the seed has been processed such as milled corn, milled maize, defatted maize, defatted destarched maize, milled sorghum, milled switchgrass, milled millet, milled foxtail millet, milled pearl millet, or any combination thereof.

### Animal Feed and Animal Feed Additives

The present invention also relates to animal feed compositions and animal feed additives comprising one or more xylanases of the invention. In an embodiment, the animal feed or animal feed additive comprises a formulating agent and one or more xylanases of the invention. In a further embodiment, the formulating agent comprises one or more of the following compounds:

glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose.

Thus the invention further relates to an animal feed additive comprising one or more polypeptides having xylanase activity and one or more vitamins, wherein the polypeptide having xylanase activity is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In an embodiment, the animal feed additive further comprises one or more enzymes, one or more microbes, one or more vitamins, one or more amino acids and/or one or more other feed ingredients.

The invention further relates to an animal feed additive comprising one or more polypeptides having xylanase activity and one or more minerals, wherein the polypeptide having xylanase activity is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In an embodiment, the animal feed additive further comprises one or more enzymes, one or more microbes, one or more vitamins, one or more amino acids and/or one or more other feed ingredients.

The invention further relates to an animal feed additive comprising one or more polypeptides having xylanase activity and one or more amino acids, wherein the polypeptide having xylanase activity is selected from the group consisting of:
(a) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 60;
(b) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 92%, at least 93%, at least 94%, at least 95%, at least 96%, at least 97%, at least 98%, at least 99% or 100% sequence identity to the polypeptide of SEQ ID NO: 12;
(d) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(e) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(f) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(g) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(h) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 42;
(i) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 48;
(j) a polypeptide having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

In an embodiment, the animal feed additive further comprises one or more enzymes, one or more microbes, one or more vitamins, one or more minerals and/or one or more other feed ingredients.

In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from defatted destarched maize (DFDSM). In one embodiment, the GH30 subfamily 8 polypeptide solubilises at least 4%, such as at least 4.5%, at least 5%, at least 5.5%, at least 6%, at least 6.5%, at least 7%, at least 7.5%, at least 8%, at least 8.5% or at least 9.0% xylan from DFDSM under the reaction conditions 20 μg GH30 polypeptide per gram DFDSM and incubation at 40° C., pH 5 for 2.5 hours.

In an embodiment, the polypeptide comprises or consists of amino acids 1 to 557 of SEQ ID NO: 60. In an embodiment, the polypeptide comprises or consists of amino acids 1 to 569 of SEQ ID NO: 63. In an embodiment, the polypeptide comprises or consists of amino acids 1 to 382 of SEQ ID NO: 6. In an embodiment, the polypeptide comprises or consists of amino acids 1 to 390 of SEQ ID NO: 9. In an embodiment, the polypeptide comprises or consists of amino acids 1 to 391 of SEQ ID NO: 12. In an embodiment, the polypeptide comprises or consists of amino acids 1 to 399 of SEQ ID NO: 15. In an embodiment, the polypeptide comprises or consists of amino acids 1 to 383 of SEQ ID NO: 18. In an embodiment, the polypeptide comprises or consists of amino acids 1 to 391 of SEQ ID NO: 21. In an embodiment, the polypeptide comprises or consists of amino acids 1 to 565 of SEQ ID NO: 24. In an embodiment, the polypeptide comprises or consists of amino acids 1 to 573 of SEQ ID NO: 27. In an embodiment, the polypeptide comprises or consists of amino acids 1 to 396 of SEQ ID NO: 30. In an embodiment, the polypeptide comprises or consists of amino acids 1 to 404 of SEQ ID NO: 33. In an embodiment, the polypeptide comprises or consists of amino acids 1 to 392 of SEQ ID NO: 36. In an embodiment, the polypeptide comprises or consists of amino acids 1 to 400 of SEQ ID NO: 39. In an embodiment, the polypeptide comprises or consists of amino acids 1 to 413 of SEQ ID NO: 42. In an embodiment, the polypeptide comprises or consists of amino acids 1 to 421 of SEQ ID NO: 45. In an embodiment, the polypeptide comprises or consists of amino acids 1 to 398 of SEQ ID NO: 48. In an embodiment, the polypeptide comprises or consists of amino acids 1 to 406 of SEQ ID NO: 51. In an embodiment, the polypeptide comprises or consists of amino acids 1 to 382 of SEQ ID NO: 54. In an embodiment, the polypeptide comprises or consists of amino acids 1 to 390 of SEQ ID NO: 57.

In a further aspect, the invention relates to an animal feed comprising the animal feed additive as described above and plant based material from the sub-family Panicoideae. In an embodiment, the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof. In a further embodiment, the the plant based material from the sub-family Panicoideae is from the seed fraction (such as endosperm and/or husk) of the plant.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

An animal feed composition according to the invention has a crude protein content of 50-800 g/kg, and furthermore comprises at least one xylanase as claimed herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein as defined above.

The animal feed composition of the invention may also contain animal protein, such as Meat and Bone Meal, Feather meal, and/or Fish Meal, typically in an amount of 0-25%. The animal feed composition of the invention may also comprise Dried Distillers Grains with Solubles (DDGS), typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) xylanase/enzyme preparation may also be added before or during the feed ingredient step. Typically a liquid xylanase/enzyme preparation comprises the xylanase of the invention optionally with a polyol, such as glycerol, ethylene glycol or propylene glycol, and is added after the pelleting step, such as by spraying the liquid formulation onto the pellets. The enzyme may also be incorporated in a feed additive or premix.

Alternatively, the xylanase can be prepared by freezing a mixture of liquid enzyme solution with a bulking agent such as ground soybean meal, and then lyophilizing the mixture.

The final enzyme concentration in the diet is within the range of 0.01-200 mg enzyme protein per kg diet, preferably between 0.05-100 mg/kg diet, more preferably 0.1-50 mg, even more preferably 0.2-20 mg enzyme protein per kg animal diet.

It is at present contemplated that the enzyme is administered in one or more of the following amounts (dosage ranges): 0.01-200; 0.05-100; 0.1-50; 0.2-20; 0.1-1; 0.2-2; 0.5-5; or 1-10;—all these ranges being in mg xylanase protein per kg feed (ppm).

For determining mg xylanase protein per kg feed, the xylanase is purified from the feed composition, and the specific activity of the purified xylanase is determined using a relevant assay (see under xylanase activity). The xylanase activity of the feed composition as such is also determined using the same assay, and on the basis of these two determinations, the dosage in mg xylanase protein per kg feed is calculated.

In a particular embodiment, the animal feed additive of the invention is intended for being included (or prescribed as having to be included) in animal diets or feed at levels of 0.01 to 10.0%; more particularly 0.05 to 5.0%; or 0.2 to 1.0% (')/0 meaning g additive per 100 g feed). This is so in particular for premixes.

The same principles apply for determining mg xylanase protein in feed additives. Of course, if a sample is available of the xylanase used for preparing the feed additive or the feed, the specific activity is determined from this sample (no need to purify the xylanase from the feed composition or the additive).

### Additional Enzymes

In another embodiment, the compositions described herein optionally include one or more enzymes. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase, lysozyme and galactosidase is described in Henrissat et al, “The carbohydrate-active enzymes database (CAZy) in 2013”, Nucl. Acids Res. (1 Jan. 2014) 42 (D1): D490-D495; see also www.cazy.org. Thus the composition of the invention may also comprise at least one other enzyme selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); arabinofuranosidase (EC 3.2.1.55); beta-xylosidase (EC 3.2.1.37); acetyl xylan esterase (EC 3.1.1.72); feruloyl esterase (EC 3.1.1.73); cellulase (EC 3.2.1.4); cellobiohydrolases (EC 3.2.1.91); beta-glucosidase (EC 3.2.1.21); pullulanase (EC 3.2.1.41), alpha-mannosidase (EC 3.2.1.24), mannanase (EC 3.2.1.25) and beta-glucanase (EC 3.2.1.4 or EC 3.2.1.6), or any mixture thereof.

In a particular embodiment, the composition of the invention comprises a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Finase® and Quantum® Blue (AB Enzymes), OptiPhos® (Huvepharma) Phyzyme® XP (Verenium/DuPont) and Axtra® PHY (DuPont). Other preferred phytases include those described in e.g. WO 98/28408, WO 00/43503, and WO 03/066847.

In a particular embodiment, the composition of the invention comprises a xylanase (EC 3.2.1.8). Examples of commercially available xylanases include Ronozyme® WX and Ronozyme® G2 (DSM Nutritional Products), Econase® XT and Barley (AB Vista), Xylathin® (Verenium), Hostazym® X (Huvepharma) and Axtra® XB (Xylanase/beta-glucanase, DuPont).

In a particular embodiment, the composition of the invention comprises a protease (EC 3.4). Examples of commercially available proteases include Ronozyme® ProAct (DSM Nutritional Products).

### Eubiotics

Eubiotics are compounds which are designed to give a healthy balance of the micro-flora in the gastrointestinal tract. Eubiotics cover a number of different feed additives, such as probiotics, prebiotics, phytogenics (essential oils) and organic acids which are described in more detail below.

### Probiotics

In an embodiment, the animal feed composition further comprises one or more additional probiotic. In a particular embodiment, the animal feed composition further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a preferred embodiment, animal feed composition further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

In a more preferred embodiment, composition, animal feed additive or animal feed further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, NRRL B-50136, NRRL B-50605, NRRL B-50606, NRRL B-50622 and PTA-7547.

In a more preferred embodiment, composition, animal feed additive or animal feed further comprises a bacterium from one or more of the following strains of Bacillus pumilus: NRRL B-50016, ATCC 700385, NRRL B-50885 or NRRL B-50886.

In a more preferred embodiment, composition, animal feed additive or animal feed further comprises a bacterium from one or more of the following strains of Bacillus lichenformis: NRRL B 50015, NRRL B-50621 or NRRL B-50623.

In a more preferred embodiment, composition, animal feed additive or animal feed further comprises a bacterium from one or more of the following strains of Bacillus amyloliquefaciens: DSM 29869, DSM 29869, NRRL B 50607, PTA-7543, PTA-7549, NRRL B-50349, NRRL B-50606, NRRL B-50013, NRRL B-50151, NRRL B-50141, NRRL B-50147 or NRRL B-50888.

The bacterial count of each of the bacterial strains in the animal feed composition is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. In a more preferred embodiment the bacterial count of each of the bacterial strains in the animal feed composition is between 1×10° and 1×10¹⁰CFU/kg of dry matter.

The bacterial count of each of the bacterial strains in the animal feed composition is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. In a more preferred embodiment the bacterial count of each of the bacterial strains in the animal feed composition is between 1×10⁹ and 1×10¹¹ CFU/animal/day.

In another embodiment, the one or more bacterial strains are present in the form of a stable spore.

Examples of commercial products are Cylactin® (DSM Nutritional Products), Alterion (Adisseo), Enviva PRO (DuPont Animal Nutrition), Syncra® (mix enzyme+probiotic, DuPont Animal Nutrition).

### Prebiotics

Prebiotics are substances that induce the growth or activity of microorganisms (e.g., bacteria and fungi) that contribute to the well-being of their host. Prebiotics are typically non-digestible fiber compounds that pass undigested through the upper part of the gastrointestinal tract and stimulate the growth or activity of advantageous bacteria that colonize the large bowel by acting as substrate for them. Normally, prebiotics increase the number or activity of bifidobacteria and lactic acid bacteria in the GI tract.

Yeast derivatives (inactivated whole yeasts or yeast cell walls) can also be considered as prebiotics. They often comprise mannan-oligosaccharids, yeast beta-glucans or protein contents and are normally derived from the cell wall of the yeast, Saccharomyces cerevisiae.

Examples of yeast products are Yang® and Agrimos (Lallemand Animal Nutrition).

### Phytogenics

Phytogenics are a group of natural growth promoters or non-antibiotic growth promoters used as feed additives, derived from herbs, spices or other plants. Phytogenics can be single substances prepared from essential oils/extracts, essential oils/extracts, single plants and mixture of plants (herbal products) or mixture of essential oils/extracts/plants (specialized products).

Examples of phytogenics are rosemary, sage, oregano, thyme, clove, and lemongrass. Examples of essential oils are thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract.

Examples of commercial products are Crina® (DSM Nutritional Products); Cinergy™ Biacid™, ProHacid™ Classic and ProHacid™ Advance™ (all Promivi/Cargill) and Envivo EO (DuPont Animal Nutrition).

### Organic Acids

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are propionic acid, formic acid, citric acid, lactic acid, sorbic acid, malic acid, acetic acid, fumaric acid, benzoic acid, butyric acid and tartaric acid or their salt (typically sodium salt). Examples of commercial products are VevoVitall® (DSM Nutritional Products), Amasil®, Luprisil®, Lupro-Grain®, Lupro-Cid®, Lupro-Mix® (BASF) and n-Butyric Acid AF (OXEA).

### Premix

In an embodiment, the animal feed may include a premix, comprising e.g. vitamins, minerals, enzymes, amino acids, preservatives, antibiotics, other feed ingredients or any combination thereof which are mixed into the animal feed.

### Amino Acids

The composition of the invention may further comprise one or more amino acids. Examples of amino acids which are used in animal feed are lysine, alanine, beta-alanine, threonine, methionine and tryptophan.

Vitamins and Minerals In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin C, vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

In a still further embodiment, the animal feed additive of the invention comprises at least one of the below vitamins, preferably to provide an in-feed-concentration within the ranges specified in the below Table 1 (for piglet diets, and broiler diets, respectively).

TABLE 1
Typical vitamin recommendations

| Vitamin | Piglet diet | | Broiler diet | |
|---|---|---|---|---|
| Vitamin A | 10,000-15,000 | IU/kg feed | 8-12,500 | IU/kg feed |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic | 20-40 | mg/kg feed | 10-18 | mg/kg feed |
| acid | | | | |
| Folic acid | 1-2 | mg/kg feed | 1-2 | mg/kg feed |
| Biotin | 0.15-0.4 | mg/kg feed | 0.15-0.3 | mg/kg feed |
| Choline | 200-400 | mg/kg feed | 300-600 | mg/kg feed |
| chloride | | | | |

### Other Feed Ingredients

The composition of the invention may further comprise colouring agents, stabilisers, growth improving additives and aroma compounds/flavourings, polyunsaturated fatty acids (PUFAs); reactive oxygen generating species, anti-microbial peptides and anti-fungal polypeptides.

Examples of colouring agents are carotenoids such as beta-carotene, astaxanthin, and lutein.

Examples of aroma compounds/flavourings are creosol, anethol, deca-, undeca-and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are 018, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

The composition of the invention may further comprise at least one amino acid. Examples of amino acids which are used in animal feed are lysine, alanine, beta-alanine, threonine, methionine and tryptophan.

### Uses

The present invention is also directed to methods for using the polypeptides having xylanase activity, or compositions thereof, for e.g. animal feed. The present invention is also directed to processes for using the polypeptides having xylanase activity, or compositions thereof, such as e.g. those described below.

### Use in Animal Feed

The present invention is also directed to methods for using the xylanases of the invention in animal feed.

The term animal includes all animals. In one embodiment, the term animal excludes humans. Examples of animals are non-ruminants, and ruminants. Ruminant animals include, for example, animals such as sheep, goats, and cattle, e.g. beef cattle, cows, and young calves. In a particular embodiment, the animal is a non-ruminant animal. Non-ruminant animals include mono-gastric animals, e.g. pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), young calves; and fish (including but not limited to salmon, trout, tilapia, catfish and carps; and crustaceans (including but not limited to shrimps and prawns).

In the use according to the invention the xylanases can be fed to the animal before, after, or simultaneously with the diet. The latter is preferred.

In a particular embodiment, the xylanase, in the form in which it is added to the feed, or when being included in a feed additive, is well-defined. Well-defined means that the xylanase preparation is at least 50% pure as determined by Size-exclusion chromatography (see Example 12 of WO 01/58275). In other particular embodiments the xylanase preparation is at least 60, 70, 80, 85, 88, 90, 92, 94, or at least 95% pure as determined by this method.

A well-defined xylanase preparation is advantageous. For instance, it is much easier to dose correctly to the feed a xylanase that is essentially free from interfering or contaminating other xylanases. The term dose correctly refers in particular to the objective of obtaining consistent and constant results, and the capability of optimizing dosage based upon the desired effect.

For the use in animal feed, however, the xylanase need not be that pure; it may e.g. include other enzymes, in which case it could be termed a xylanase preparation.

The xylanase preparation can be (a) added directly to the feed, or (b) it can be used in the production of one or more intermediate compositions such as feed additives or premixes that is subsequently added to the feed (or used in a treatment process). The degree of purity described above refers to the purity of the original xylanase preparation, whether used according to (a) or (b) above.

### Preferred Embodiments of the Invention

Preferred embodiments of the invention are described in the set of items below.
1. A method of solubilising xylan from plant based material, comprising treating plant based material with a GH30 subfamily 8 polypeptide having xylanase activity, wherein the GH30 subfamily 8 polypeptide having xylanase activity is selected from the group consisting of:
   (a) a polypeptide having at least 80% sequence identity to the polypeptide of SEQ ID NO: 60;
   (b) a polypeptide having at least 80% sequence identity to the polypeptide of SEQ ID NO: 6;
   (c) a polypeptide having at least 92% sequence identity to the polypeptide of SEQ ID NO: 12;
   (d) a polypeptide having at least 80% sequence identity to the polypeptide of SEQ ID NO: 18;
   (e) a polypeptide having at least 80% sequence identity to the polypeptide of SEQ ID NO: 24;
   (f) a polypeptide having at least 80% sequence identity to the polypeptide of SEQ ID NO: 30;
   (g) a polypeptide having at least 80% sequence identity to the polypeptide of SEQ ID NO: 36;
   (h) a polypeptide having at least 85% sequence identity to the polypeptide of SEQ ID NO: 42;
   (i) a polypeptide having at least 80% sequence identity to the polypeptide of SEQ ID NO: 48;
   (j) a polypeptide having at least 80% sequence identity to the polypeptide of SEQ ID NO: 54;
   (k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
   (l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions; (m) a polypeptide comprising the polypeptide of (a), (b), (c), (d), (e), (f), (g), (h), (i), (j), (k) or (l) and a N-terminal and/or C-terminal His-tag and/or HQ-tag; and
   (n) a fragment of a polypeptide of (a), (b), (c), (d), (e), (f), (g), (h), (i), (j), (k), (l) or (m) having at least 90% of the length of the mature polypeptide.
2. The method of item 1, wherein the polypeptide comprises the motif YXWWY[I/L]RRXYG (SEQ ID NO: 1).
3. The method of any of items 1 to 2, wherein the polypeptide is selected from the group consisting of:
   (a) a polypeptide having at least 85% sequence identity to the polypeptide of SEQ ID NO: 60;
   (b) a polypeptide having at least 85% sequence identity to the polypeptide of SEQ ID NO: 6;
   (c) a polypeptide having at least 92% sequence identity to the polypeptide of SEQ ID NO: 12;
   (d) a polypeptide having at least 85% sequence identity to the polypeptide of SEQ ID NO: 18;
   (e) a polypeptide having at least 85% sequence identity to the polypeptide of SEQ ID NO: 24;
   (f) a polypeptide having at least 85% sequence identity to the polypeptide of SEQ ID NO: 30;
   (g) a polypeptide having at least 85% sequence identity to the polypeptide of SEQ ID NO: 36;
   (h) a polypeptide having at least 85% sequence identity to the polypeptide of SEQ ID NO: 42;
   (i) a polypeptide having at least 85% sequence identity to the polypeptide of SEQ ID NO: 48; and
   (j) a polypeptide having at least 85% sequence identity to the polypeptide of SEQ ID NO: 54.
4. The method of any of items 1 to 2, wherein the polypeptide is selected from the group consisting of:
   (a) a polypeptide having at least 90% sequence identity to the polypeptide of SEQ ID NO: 60;
   (b) a polypeptide having at least 90% sequence identity to the polypeptide of SEQ ID NO: 6;
   (c) a polypeptide having at least 92% sequence identity to the polypeptide of SEQ ID NO: 12;
   (d) a polypeptide having at least 90% sequence identity to the polypeptide of SEQ ID NO: 18;
   (e) a polypeptide having at least 90% sequence identity to the polypeptide of SEQ ID NO: 24;
   (f) a polypeptide having at least 90% sequence identity to the polypeptide of SEQ ID NO: 30;
   (g) a polypeptide having at least 90% sequence identity to the polypeptide of SEQ ID NO: 36;
   (h) a polypeptide having at least 90% sequence identity to the polypeptide of SEQ ID NO: 42;
   (i) a polypeptide having at least 90% sequence identity to the polypeptide of SEQ ID NO: 48;
   (j) a polypeptide having at least 90% sequence identity to the polypeptide of SEQ ID NO: 54;
   (k) a variant of the polypeptide of SEQ ID NO: 6, SEQ ID NO: 18, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 positions; and
   (l) a variant of the polypeptide of SEQ ID NO: 60 or SEQ ID NO: 24 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions.
5. The method of any of items 1 to 2, wherein the polypeptide is selected from the group consisting of:
   (a) a polypeptide having at least 95% sequence identity to the polypeptide of SEQ ID NO: 60;
   (b) a polypeptide having at least 95% sequence identity to the polypeptide of SEQ ID NO: 6;
   (c) a polypeptide having at least 95% sequence identity to the polypeptide of SEQ ID NO: 12;
   (d) a polypeptide having at least 95% sequence identity to the polypeptide of SEQ ID NO: 18;
   (e) a polypeptide having at least 95% sequence identity to the polypeptide of SEQ ID NO: 24;
   (f) a polypeptide having at least 95% sequence identity to the polypeptide of SEQ ID NO: 30;
   (g) a polypeptide having at least 95% sequence identity to the polypeptide of SEQ ID NO: 36;
   (h) a polypeptide having at least 95% sequence identity to the polypeptide of SEQ ID NO: 42;
   (i) a polypeptide having at least 95% sequence identity to the polypeptide of SEQ ID NO: 48;
   (j) a polypeptide having at least 95% sequence identity to the polypeptide of SEQ ID NO: 54;
   (k) a variant of the polypeptide of SEQ ID NO: 6, SEQ ID NO: 18, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18 or 19 positions; and
   (l) a variant of the polypeptide of SEQ ID NO: 60 or SEQ ID NO: 24 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26 or 27 positions.
6. The method of any of items 1 to 2, wherein the polypeptide comprises or consists of amino acids 1 to 557 of SEQ ID NO: 60, amino acids 1 to 569 of SEQ ID NO: 63, amino acids 1 to 382 of SEQ ID NO: 6, amino acids 1 to 390 of SEQ ID NO: 9, amino acids 1 to 391 of SEQ ID NO: 12, amino acids 1 to 399 of SEQ ID NO: 15, amino acids 1 to 383 of SEQ ID NO: 18, amino acids 1 to 391 of SEQ ID NO: 21, amino acids 1 to 565 of SEQ ID NO: 24, amino acids 1 to 573 of SEQ ID NO: 27, amino acids 1 to 396 of SEQ ID NO: 30, amino acids 1 to 404 of SEQ ID NO: 33, amino acids 1 to 392 of SEQ ID NO: 36, amino acids 1 to 400 of SEQ ID NO: 39, amino acids 1 to 413 of SEQ ID NO: 42, amino acids 1 to 421 of SEQ ID NO: 45, amino acids 1 to 398 of SEQ ID NO: 48, amino acids 1 to 406 of SEQ ID NO: 51, amino acids 1 to 382 of SEQ ID NO: 54, amino acids 1 to 390 of SEQ ID NO: 57.
7. The method of any of items 1 to 6 wherein the percentage solubilised xylan is at least 4% when the method is performed under the reaction conditions 20 μg GH30 polypeptide per gram defatted destarched maize (DFDSM) and incubation at 40° C., pH 5 for 2.5 hours.
8 The method of any of items 1 to 8, wherein the plant based material is from the sub-family Panicoideae, preferably maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof.
9. The method of any of items 1 to 8, wherein the plant based material is from the seed fraction (such as endosperm and/or husk) of the plant.
10. A granule comprising one or more GH30 subfamily 8 polypeptides having xylanase activity, wherein the GH30 subfamily 8 polypeptide having xylanase activity is selected from the group consisting of:
   (a) a polypeptide having at least 80% sequence identity to the polypeptide of SEQ ID NO: 60;
   (b) a polypeptide having at least 80% sequence identity to the polypeptide of SEQ ID NO: 6;
   (c) a polypeptide having at least 92% sequence identity to the polypeptide of SEQ ID NO: 12;
   (d) a polypeptide having at least 80% sequence identity to the polypeptide of SEQ ID NO: 18;
   (e) a polypeptide having at least 80% sequence identity to the polypeptide of SEQ ID NO: 24;
   (f) a polypeptide having at least 80% sequence identity to the polypeptide of SEQ ID NO: 30;
   (g) a polypeptide having at least 80% sequence identity to the polypeptide of SEQ ID NO: 36;
   (h) a polypeptide having at least 85% sequence identity to the polypeptide of SEQ ID NO: 42;
   (i) a polypeptide having at least 80% sequence identity to the polypeptide of SEQ ID NO: 48;
   (j) a polypeptide having at least 80% sequence identity to the polypeptide of SEQ ID NO: 54;
   (k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
   (l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions; (m) a polypeptide comprising the polypeptide of (a), (b), (c), (d), (e), (f), (g), (h), (i), (j), (k) or (l) and a N-terminal and/or C-terminal His-tag and/or HQ-tag; and
   (n) a fragment of a polypeptide of (a), (b), (c), (d), (e), (f), (g), (h), (i), (j), (k), (l) or (m) having at least 90% of the length of the mature polypeptide.
11. The granule of item 10, wherein the polypeptide comprises the motif YXWWY[I/L]RRXYG (SEQ ID NO: 1).
12. The granule of any of items 10 to 11, wherein the polypeptide is selected from the group consisting of:
   (a) a polypeptide having at least 85% sequence identity to the polypeptide of SEQ ID NO: 60;
   (b) a polypeptide having at least 85% sequence identity to the polypeptide of SEQ ID NO: 6;
   (c) a polypeptide having at least 92% sequence identity to the polypeptide of SEQ ID NO: 12;
   (d) a polypeptide having at least 85% sequence identity to the polypeptide of SEQ ID NO: 18;
   (e) a polypeptide having at least 85% sequence identity to the polypeptide of SEQ ID NO: 24;
   (f) a polypeptide having at least 85% sequence identity to the polypeptide of SEQ ID NO: 30;
   (g) a polypeptide having at least 85% sequence identity to the polypeptide of SEQ ID NO: 36;
   (h) a polypeptide having at least 85% sequence identity to the polypeptide of SEQ ID NO: 42;
   (i) a polypeptide having at least 85% sequence identity to the polypeptide of SEQ ID NO: 48; and
   (j) a polypeptide having at least 85% sequence identity to the polypeptide of SEQ ID NO: 54.
13. The granule of any of items 10 to 11, wherein the polypeptide is selected from the group consisting of:
   (a) a polypeptide having at least 90% sequence identity to the polypeptide of SEQ ID NO: 60;
   (b) a polypeptide having at least 90% sequence identity to the polypeptide of SEQ ID NO: 6;
   (c) a polypeptide having at least 92% sequence identity to the polypeptide of SEQ ID NO: 12;
   (d) a polypeptide having at least 90% sequence identity to the polypeptide of SEQ ID NO: 18;
   (e) a polypeptide having at least 90% sequence identity to the polypeptide of SEQ ID NO: 24;
   (f) a polypeptide having at least 90% sequence identity to the polypeptide of SEQ ID NO: 30;
   (g) a polypeptide having at least 90% sequence identity to the polypeptide of SEQ ID NO: 36;
   (h) a polypeptide having at least 90% sequence identity to the polypeptide of SEQ ID NO: 42;
   (i) a polypeptide having at least 90% sequence identity to the polypeptide of SEQ ID NO: 48;
   (j) a polypeptide having at least 90% sequence identity to the polypeptide of SEQ ID NO: 54;
   (k) a variant of the polypeptide of SEQ ID NO: 6, SEQ ID NO: 18, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 positions; and
   (l) a variant of the polypeptide of SEQ ID NO: 60 or SEQ ID NO: 24 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions.
14. The granule of any of items 10 to 11, wherein the polypeptide is selected from the group consisting of:
   (a) a polypeptide having at least 95% sequence identity to the polypeptide of SEQ ID NO: 60;
   (b) a polypeptide having at least 95% sequence identity to the polypeptide of SEQ ID NO: 6;
   (c) a polypeptide having at least 95% sequence identity to the polypeptide of SEQ ID NO: 12;
   (d) a polypeptide having at least 95% sequence identity to the polypeptide of SEQ ID NO: 18;
   (e) a polypeptide having at least 95% sequence identity to the polypeptide of SEQ ID NO: 24;
   (f) a polypeptide having at least 95% sequence identity to the polypeptide of SEQ ID NO: 30;
   (g) a polypeptide having at least 95% sequence identity to the polypeptide of SEQ ID NO: 36;
   (h) a polypeptide having at least 95% sequence identity to the polypeptide of SEQ ID NO: 42;
   (i) a polypeptide having at least 95% sequence identity to the polypeptide of SEQ ID NO: 48;
   (j) a polypeptide having at least 95% sequence identity to the polypeptide of SEQ ID NO: 54;
   (k) a variant of the polypeptide of SEQ ID NO: 6, SEQ ID NO: 18, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18 or 19 positions; and
   (l) a variant of the polypeptide of SEQ ID NO: 60 or SEQ ID NO: 24 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26 or 27 positions.
15. The granule of any of items 10 to 11, wherein the polypeptide comprises or consists of amino acids 1 to 557 of SEQ ID NO: 60, amino acids 1 to 569 of SEQ ID NO: 63, amino acids 1 to 382 of SEQ ID NO: 6, amino acids 1 to 390 of SEQ ID NO: 9, amino acids 1 to 391 of SEQ ID NO: 12, amino acids 1 to 399 of SEQ ID NO: 15, amino acids 1 to 383 of SEQ ID NO: 18, amino acids 1 to 391 of SEQ ID NO: 21, amino acids 1 to 565 of SEQ ID NO: 24, amino acids 1 to 573 of SEQ ID NO: 27, amino acids 1 to 396 of SEQ ID NO: 30, amino acids 1 to 404 of SEQ ID NO: 33, amino acids 1 to 392 of SEQ ID NO: 36, amino acids 1 to 400 of SEQ ID NO: 39, amino acids 1 to 413 of SEQ ID NO: 42, amino acids 1 to 421 of SEQ ID NO: 45, amino acids 1 to 398 of SEQ ID NO: 48, amino acids 1 to 406 of SEQ ID NO: 51, amino acids 1 to 382 of SEQ ID NO: 54, amino acids 1 to 390 of SEQ ID NO: 57.
16. The granule of any of items 10 to 15, wherein the polypeptide solubilises at least 4% xylan from defatted destarched maize (DFDSM).
17 The granule of any of items 10 to 16, wherein the polypeptide solubilises at least 4% xylan from defatted destarched maize (DFDSM) under the reaction conditions 20 μg GH30 polypeptide per gram DFDSM and incubation at 40° C., pH 5 for 2.5 hours.
18. The granule of any of items 16 to 17, wherein the polypeptide solubilises at least 4.5%, such as at least 5%, at least 5.5%, at least 6%, at least 6.5%, at least 7%, at least 7.5%, at least 8%, at least 8.5% or at least 9.0% xylan from DFDSM.
19. The granule of any of items 10 to 18, wherein the granule comprises one or more formulating agents.
20. The granule of item 19, wherein the formulating agent is selected from the list consisting of glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose, preferably selected from the list consisting of 1,2-propylene glycol, 1,3-propylene glycol, sodium sulfate, dextrin, cellulose, sodium thiosulfate, kaolin and calcium carbonate.
21. The granule of any of items 10 to 20, wherein the granule comprises a core particle and one or more coatings.
22. The granule of item 21, wherein the coating comprises salt and/or wax and/or flour.
23. The granule of any of items 10 to 22 further comprising one or more additional enzymes.
24. The granule of item 23, wherein the one or more additional enzymes is selected from the group consisting of phytase, xylanase, galactanase, alpha-galactosidase, beta-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.
23. The granule of any of items 10 to 24 further comprising one or more probiotics.
24. The granule of item 25, wherein the one or more probiotics is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof.
25. An isolated polypeptide having xylanase activity, selected from the group consisting of:
   (a) a polypeptide having at least 80% sequence identity to the polypeptide of SEQ ID NO: 6;
   (b) a polypeptide having at least 92% sequence identity to the polypeptide of SEQ ID NO: 12;
   (c) a polypeptide having at least 86% sequence identity to the polypeptide of SEQ ID NO: 30;
   (d) a polypeptide having at least 90% sequence identity to the polypeptide of SEQ ID NO: 36;
   (e) a polypeptide having at least 85% sequence identity to the polypeptide of SEQ ID NO: 42;
   (f) a polypeptide having at least 80% sequence identity to the polypeptide of SEQ ID NO: 48;
   (g) a polypeptide having at least 80% sequence identity to the polypeptide of SEQ ID NO: 54;
   (h) a polypeptide encoded by a polynucleotide that hybridizes under high stringency conditions, or very high stringency conditions with
      (i) the mature polypeptide coding sequence of SEQ ID NO: 4,
      (ii) the mature polypeptide coding sequence of SEQ ID NO: 10,
      (iii) the mature polypeptide coding sequence of SEQ ID NO: 28,
      (iv) the mature polypeptide coding sequence of SEQ ID NO: 34,
      (v) the mature polypeptide coding sequence of SEQ ID NO: 40,
      (vi) the mature polypeptide coding sequence of SEQ ID NO: 46,
      (vii) the mature polypeptide coding sequence of SEQ ID NO: 52,
      (viii) the full-length complementary strand of (i), (ii), (iii), (iv), (v), (vi) or (vii);
   (i) a polypeptide encoded by a polynucleotide having at least 80% sequence identity to the mature polypeptide coding sequence of SEQ ID NO: 4;
   (j) a polypeptide encoded by a polynucleotide having at least 90% sequence identity to the mature polypeptide coding sequence of SEQ ID NO: 10;
   (k) a polypeptide encoded by a polynucleotide having at least 86% sequence identity to the mature polypeptide coding sequence of SEQ ID NO: 28;
   (l) a polypeptide encoded by a polynucleotide having at least 90% sequence identity to the mature polypeptide coding sequence of SEQ ID NO: 34;
   (m) a polypeptide encoded by a polynucleotide having at least 85% sequence identity to the mature polypeptide coding sequence of SEQ ID NO: 40;
   (n) a polypeptide encoded by a polynucleotide having at least 80% sequence identity to the mature polypeptide coding sequence of SEQ ID NO: 46;
   (o) a polypeptide encoded by a polynucleotide having at least 80% sequence identity to the mature polypeptide coding sequence of SEQ ID NO: 52;
   (p) a variant of the polypeptide of SEQ ID NO: 6, SEQ ID NO: 18, SEQ ID NO: 30, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
   (q) a variant of the polypeptide of SEQ ID NO: 12 or SEQ ID NO: 36 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
   (r) a polypeptide comprising the polypeptide of (a), (b), (c), (d), (e), (f), (g), (h), (i), (j), (k), (l), (m), (n), (o), (p) or (q) and a N-terminal and/or C-terminal His-tag and/or HQ-tag; and
   (s) a fragment of a polypeptide of (a), (b), (c), (d), (e), (f), (g), (h), (i), (j), (k), (l), (m), (n), (o), (p), (q) or (r) having xylanase activity and having at least 90% of the length of the mature polypeptide.
26. The polypeptide of item 25, wherein the polypeptide comprises or consists of amino acids 1 to 382 of SEQ ID NO: 6, amino acids 1 to 390 of SEQ ID NO: 9, amino acids 1 to 391 of SEQ ID NO: 12, amino acids 1 to 399 of SEQ ID NO: 15, amino acids 1 to 383 of SEQ ID NO: 18, amino acids 1 to 391 of SEQ ID NO: 21, amino acids 1 to 396 of SEQ ID NO: 30, amino acids 1 to 404 of SEQ ID NO: 33, amino acids 1 to 392 of SEQ ID NO: 36, amino acids 1 to 400 of SEQ ID NO: 39, amino acids 1 to 413 of SEQ ID NO: 42, amino acids 1 to 421 of SEQ ID NO: 45, amino acids 1 to 398 of SEQ ID NO: 48, amino acids 1 to 406 of SEQ ID NO: 51, amino acids 1 to 382 of SEQ ID NO: 54, amino acids 1 to 390 of SEQ ID NO: 57.
27. A composition comprising the polypeptide of any of items 25 to 26.
28. The composition of item 27 further comprising one or more formulating agents.
29. The composition of item 28 wherein the formulating agent comprises one or more of the following compounds: glycerol, ethylene glycol, 1, 2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose.
30. The composition of any of items 27 to 29 further comprising one or more additional enzymes.
31. The composition of item 30, wherein the one or more additional enzymes is selected from the group consisting of phytase, xylanase, galactanase, alpha-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.
32. The composition of any of items 27 to 31 further comprising one or more microbes.
33. The composition of item 32, wherein the one or more microbes is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof.
34. The composition of any of items 27 to 33 further comprising plant based material from the sub-family Panicoideae.
35. The composition of item 34, wherein the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof.
36. The composition of any of items 27 to 35, wherein the plant based material from the sub-family Panicoideae is from the seed fraction (such as endosperm and/or husk) of the plant.
37. An animal feed additive comprising one or more GH30 subfamily 8 polypeptides having xylanase activity, wherein the polypeptide having xylanase activity is selected from the group consisting of:
   (a) a polypeptide having at least 80% sequence identity to the polypeptide of SEQ ID NO: 60;
   (b) a polypeptide having at least 80% sequence identity to the polypeptide of SEQ ID NO: 6;
   (c) a polypeptide having at least 92% sequence identity to the polypeptide of SEQ ID NO: 12;
   (d) a polypeptide having at least 80% sequence identity to the polypeptide of SEQ ID NO: 18;
   (e) a polypeptide having at least 80% sequence identity to the polypeptide of SEQ ID NO: 24;
   (f) a polypeptide having at least 80% sequence identity to the polypeptide of SEQ ID NO: 30;
   (g) a polypeptide having at least 80% sequence identity to the polypeptide of SEQ ID NO: 36;
   (h) a polypeptide having at least 85% sequence identity to the polypeptide of SEQ ID NO: 42;
   (i) a polypeptide having at least 80% sequence identity to the polypeptide of SEQ ID NO: 48;
   (j) a polypeptide having at least 80% sequence identity to the polypeptide of SEQ ID NO: 54;
   (k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
   (l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions; (m) a polypeptide comprising the polypeptide of (a), (b), (c), (d), (e), (f), (g), (h), (i), (j), (k) or (l) and a N-terminal and/or C-terminal His-tag and/or HQ-tag; and
   (n) a fragment of a polypeptide of (a), (b), (c), (d), (e), (f), (g), (h), (i), (j), (k), (l) or (m) having at least 90% of the length of the mature polypeptide.
38. The animal feed additive of item 37, wherein the polypeptide comprises or consists of amino acids 1 to 557 of SEQ ID NO: 60, amino acids 1 to 569 of SEQ ID NO: 63, amino acids 1 to 382 of SEQ ID NO: 6, amino acids 1 to 390 of SEQ ID NO: 9, amino acids 1 to 391 of SEQ ID NO: 12, amino acids 1 to 399 of SEQ ID NO: 15, amino acids 1 to 383 of SEQ ID NO: 18, amino acids 1 to 391 of SEQ ID NO: 21, amino acids 1 to 565 of SEQ ID NO: 24, amino acids 1 to 573 of SEQ ID NO: 27, amino acids 1 to 396 of SEQ ID NO: 30, amino acids 1 to 404 of SEQ ID NO: 33, amino acids 1 to 392 of SEQ ID NO: 36, amino acids 1 to 400 of SEQ ID NO: 39, amino acids 1 to 413 of SEQ ID NO: 42, amino acids 1 to 421 of SEQ ID NO: 45, amino acids 1 to 398 of SEQ ID NO: 48, amino acids 1 to 406 of SEQ ID NO: 51, amino acids 1 to 382 of SEQ ID NO: 54, amino acids 1 to 390 of SEQ ID NO: 57.
39. The animal feed additive of any of items 37 to 38 further comprising one or more components selected from the list consisting of:
   one or more vitamins;
   one or more minerals;
   one or more amino acids;
   one or more prebiotics;
   one or more organic acids; and
   one or more other feed ingredients.
40. The animal feed additive of any of items 37 to 39 further comprising one or more additional enzymes.
41. The animal feed additive of item 40, wherein the one or more additional enzymes is selected from the group consisting of phytase, lysozyme, galactanase, alpha-galactosidase, beta-galactosidase, protease, phospholipase Al, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.
42. The animal feed additive of any of items 37 to 41 further comprising one or more probiotics.
43. The animal feed additive of item 42, wherein the one or more probiotics is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof.
44. The animal feed additive of any of items 37 to 43 further comprising one or more phytogenics.
45. The animal feed additive of item 44, wherein the phytogenic is selected from the group consisting of rosemary, sage, oregano, thyme, clove, lemongrass, essential oils, thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract or any combination thereof.
46. An animal feed comprising the polypeptide of any of items 25 to 26, the granule of any of items 10 to 24, the composition of any of items 27 to 36, or the animal feed additive of any of items 37 to 45 and plant based material from the sub-family Panicoideae.
47. The animal feed of item 46, wherein the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof.
48. The animal feed of any of items 46 to 47, wherein the plant based material from the sub-family Panicoideae is from the seed fraction (such as endosperm and/or husk) of the plant.
49. A pelleted animal feed comprising the polypeptide of any of items 25 to 26, the granule of any of items 10 to 24, the composition of any of items 27 to 36, or the animal feed additive of any of items 37 to 45 and plant based material from the sub-family Panicoideae.
50. The pelleted animal feed of item 49, wherein the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof.
51. The pelleted animal feed of any of items 49 to 50, wherein the plant based material from the sub-family Panicoideae is from the seed fraction (such as endosperm and/or husk) of the plant.
52. A method of improving one or more performance parameters of an animal comprising administering to one or more animals the polypeptide of any of items 25 to 26, the granule of any of items 10 to 24, the composition of any of items 27 to 36, or the animal feed additive of any of items 37 to 45.
53. The method of item 52, wherein improving the performance of an animal means improved body weight gain, improved European Production Efficiency Factor (EPEF) and/or improved FCR.
54. A method of preparing an animal feed comprising mixing the polypeptide of any of items 25 to 26, the granule of any of items 10 to 24, the composition of any of items 27 to 36, or the animal feed additive of any of items 37 to 45 with plant based material from the sub-family Panicoideae.
55. A method for improving the nutritional value of an animal feed comprising plant based material from the sub-family Panicoideae, comprising adding to the feed the polypeptide of any of items 25 to 26, the granule of any of items 10 to 24, the composition of any of items 27 to 36, or the animal feed additive of any of items 37 to 45.
56. A method of solubilising xylan from plant based material, comprising treating plant based material from the sub-family Panicoideae with the polypeptide of any of items 25 to 26, the granule of any of items 10 to 24, the composition of any of items 27 to 36, or the animal feed additive of any of items 37 to 45.
57. A method of releasing starch from plant based material, comprising treating plant based material from the sub-family Panicoideae with the polypeptide of any of items 25 to 26, the granule of any of items 10 to 24, the composition of any of items 27 to 36, or the animal feed additive of any of items 37 to 45.
58. The method of any of items 49 to 54, wherein the plant based material from the sub-family Panicoideae is maize, corn, sorghum, switchgrass, millet, pearl millet, foxtail millet or in a processed form such as milled corn, milled maize, defatted maize, defatted destarched maize, milled sorghum, milled switchgrass, milled millet, milled foxtail millet, milled pearl millet, or any combination thereof.
59. The method of any of items 52 to 58, wherein the plant based material from the sub-family Panicoideae is from the seed fraction (such as endosperm and/or husk) of the plant.
60. A polynucleotide encoding the polypeptide of any of items 25 to 26.
61. A nucleic acid construct or expression vector comprising the polynucleotide of item 60 operably linked to one or more control sequences that direct the production of the polypeptide in an expression host.
62. A recombinant host cell comprising the polynucleotide of item 60 operably linked to one or more control sequences that direct the production of the polypeptide.
63. A method of producing the polypeptide of any of items 25 to 26, comprising:
   (a) cultivating a cell, which in its wild-type form produces the polypeptide, under conditions conductive for production of the polypeptide; and
   (b) recovering the polypeptide.
64. A method of producing the polypeptide of any of items 25 to 26, comprising:
   (a) cultivating a host cell of item 62 under conditions conducive for production of the polypeptide; and
   (b) recovering the polypeptide.
65. A transgenic plant, plant part or plant cell transformed with a polynucleotide encoding the polypeptide of any of items 25 to 26.
66. A whole broth formulation or cell culture composition comprising a polypeptide of any of items 25 to 26.
67. Use of the polypeptide of any of items 25 to 26, the granule of any of items 10 to 24, the composition of any of items 27 to 36, or the animal feed additive of any of items 37 to 45:
   in animal feed;
   in animal feed additives;
   in the preparation of a composition for use in animal feed;
   for improving the nutritional value of an animal feed;
   for increasing digestibility of the animal feed;
   for improving one or more performance parameters in an animal;
   for solubilising xylan from plant based material of the sub-family Panicoideae; and/or
   for releasing starch from plant based material of the sub-family Panicoideae.

The present invention is further described by the following examples that should not be construed as limiting the scope of the invention.

### EXAMPLES

Strains

TABLE 2
Isolation of strains

| | | | | | SEQ ID NO |
|---|---|---|---|---|---|
| | | | | | of gene/ |
| Strain | Source | Country | Year | Collection | polypeptide |
| Pseudoalteromonas | Marine | Denmark | 2010 | | 4/5 |
| tetraodonis | sediment | | | | |
| Paenibacillus sp- | Manure | Denmark | 2013 | | 10/11 |
| 19179 | | | | | |
| Pectobacterium | Agricultural | South | 2012 | | 16/17 |
| carotovorum subsp. | sample | Korea | | | |
| carotovorum PCC21 | | | | | |
| Ruminococcus sp. | Isolated from | Denmark | 2013 | MetaHit | 22/23 |
| CAG:330 | human gut | | | metagenome | |
| | | | | derived | |
| | | | | species, DTU. | |
| Streptomyces sp- | Soil sample | Canada | 1993 | | 28/29 |
| 62627 | | | | | |
| Clostridium | Isolated from | USA | 2001 | DSM 13864 | 34/35 |
| saccharobutylicum | soy bean | | | | |
| Paenibacillus | Soil from | South | 2006 | KCTC 13020T | 40/41 |
| panacisoli | ginseng field | Korea | | LMG 23405T | |
| Human Stool | Human gut | USA | 2011 | Human | 46/47 |
| metagenome | | | | Microbiome | |
| subject 159268001 | | | | Project | |
| Vibrio rhizosphaerae | Rhizosphere | India | 2007 | DSM 18581 | 52/53 |
| | of mangrove- | | | | |
| | associated | | | | |
| | wild rice | | | | |
| Clostridium | Garden soil | Connecticut | 1924 | ATCC 824 | 58/59 |
| acetobutylicum | | USA | | | |

Genome sequencing, the subsequent assembly of reads and the gene discovery (i.e. annotation of gene functions) is known to the person skilled in the art and the service can be purchased commercially.
Substrates

### Preparation of Destarched Maize (DSM)

107 kg of milled maize (<10 mm) was mixed in a tank with 253 kg of tap water at 53° C. to make a slurry. The temperature of the slurry was 47° C. and the pH 5.9. The pH was adjusted to 6.15 with 1 L of 1 N NaOH and the tank was then heated to 95° C. 1.119 kg of Termamyl® alpha-amylase (Novozymes NS, Bagsvaerd, Denmark) was added at 52° C. and incubated for 80 minutes at 95° C. The pH measured at the end of the incubation was 6.17. Cold tap water was added to the slurry and the slurry was centrifuged and decanted 3 times using a Wesffalia decanter CA-225-110 (4950±10 rμm, flow −6001/h) giving 64.5kg of sludge. The sludge was then collected, frozen and freeze-dried to give 17.1 kg of destarched maize (DSM).

### Preparation of Defatted Destarched Maize (DFDSM)

500mL acetone was added to 100 gram of destarched maize, prepared as described above. The slurry was stirred for 5 minutes and allowed to settle. The acetone was decanted and the procedure was repeated 2 times. The residue was air dried overnight to give defatted destarched maize (DFDSM) which was stored at room temperature.

### Preparation of Destarched Sorghum

Whole sorghum seeds were milled and sieved and a fraction below 0.5 mm was used for further processing. The sieved fraction was suspended in 25 mM NaOAc pH 5.5 at 20% dry matter and destarched. The destarching involved a first step at 85° C. with 500 ppm Termamyl SC alpha-amylase (Novozymes NS, Bagsvaerd, Denmark) for 20 min followed by an overnight incubation using 250 ppm Attenuzyme Flex (Novozymes N S, Bagsvaerd, Denmark) at 65° C. The slurry was centrifuged and the liquid decanted. After this another destarching was made using by adding MilliQ water and 200 ppm Termamyl SC and 200 ppm Attenuzyme Flex and incubating overnight at 65° C.

The sorghum fiber was separated from the liquid by vacuum filtration through a Whatman F glass fiber filter. The filter cake was then washed several times with excess of water to remove soluble sugars. Finally the destarched sorghum fiber was dried in an oven at 65° C. and the dry fiber milled quickly in a coffee grinder so that the particle size was in general less than 1 mm.

### Xylose Solubilization Assay

The activity of GH30_8 xylanases towards defatted destrached Maize (DFDSM) was measured by High-Performance Anion-Exchange Chromatography with Pulsed Amperometric Detection (HPAE-PAD). 2% (w/w) DFDSM suspension was prepared in 100 mM sodium acetate, 5mM CaCl₂, pH 5 and allowed to hydrate for 30 min at room temperature under gently stirring. After hydration, 200 pl substrate suspension was pipetted into a 96 well plate and mixed with 20 μl enzyme solution to obtain a final enzyme concentration of 20 PPM relative to substrate (20 μg enzyme/g substrate). The enzyme/substrate mixtures were left for hydrolysis in 2.5 h at 40° C. under gently agitation (500 RPM) in a plate incubator (Biosan PST-100 HL). After enzymatic hydrolysis, the enzyme/substrate plates were centrifuged for 10 min at 3000 RPM and 50 μl supernatant (hydrolysate) was mixed with 100 μl 1.6 M HCl and transferred to 300 μl PCR tubes and left for acid hydrolysis for 40 min at 90° C. in a PCR machine. The purpose of the acid hydrolysis is to convert soluble polysaccharides, released by the GH30 xylanase, into mono-saccharides, which can be quantified using HPAE-PAD. Samples were neutralized with 125 μl 1.4 M NAOH after acid hydrolysis and mounted on the HPAE-PAD for mono-saccharide analysis (xylose, arabinose and glucose) (Dionex ICS-3000 using a CarboPac PA1 column). Appropriate calibration curves were made using mono-saccharides stock solutions which were subjected to the same procedure of acid hydrolysis as the samples. The percentage xylose solubilized was calculated according to the equation:

$$\% Xylose solubilized = \frac{\text{[Xylose]} * V * MW}{Xxyl * Msub}$$

Where [xylose] denotes the concentration of xylose in the supernatant measured by HPAE-PAD, V the volume of the sample, MW, the molecular weight of internal xylose in arabino-xylan (132 g/mol), Xxyl, the fraction of xylose in DFDSM (0.102) and Msub, the mass of DFDSM in the sample.

### Example 1

### Expression of a GH30_8 Xylanase from Clostridium Acetobutylicum (SEQ ID NO: 63)

A linear integration vector-system was used for the expression cloning of a GH30 xylanase from Clostridium acetobutylicum (SEQ ID NO: 58). The linear integration construct was a PCR fusion product made by fusion of the gene between two Bacillus subtilis homologous chromosomal regions along with a strong promoter and a chloramphenicol resistance marker. The fusion was made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68). The SOE PCR method is also described in patent application WO 2003/095658. The gene (SEQ ID NO: 61) was expressed under the control of a triple promoter system (as described in WO 1999/43835), consisting of the promoters from Bacillus licheniformis alpha-amylase gene (amyL), Bacillus amyloliquefaciens alpha-amylase gene (amyQ), and the Bacillus thuringiensis crylliA promoter including stabilizing sequence. The gene coding for chloramphenicol acetyl-transferase was used as marker (described in e.g. Diderichsen, B.; Poulsen, G. B.; Joergensen, S. T. 1993, Plasmid, “A useful cloning vector for Bacillus subtilis” 30:312. The final gene constructs were integrated on the Bacillus chromosome by homologous recombination. The gene encoding the GH30 xylanase from Clostridium acetobutylicum was amplified from chromosomal DNA of the strain Clostridium acetobutylicum with gene specific primers containing overhang to the two flanking fragments. The upstream and downstream flanking fragments were amplified from genomic DNA of the strain iMB1361 (described in patent application WO 2003095658). The 2 linear vector fragments and the gene fragment was subjected to a Splicing by Overlap Extension (SOE) PCR reaction to assemble the 3 fragments into one linear vector construct. An aliquot of the PCR product was transformed into Bacillus subtilis. Transformants were selected on LB plates supplemented with 6 μg of chloramphenicol per ml. A recombinant Bacillus subtilis clone containing the integrated expression construct was cultivated on a rotary shaking table in 500 ml baffled Erlenmeyer flasks each containing 100 ml yeast extract-based media. After 3-5 days cultivation time at 30° C. to 37° C., enzyme containing supernatants were harvested by centrifugation and the enzymes were purified as described in example 2.

### Example 2

### Purification of the GH30_8 Xylanase from Clostridium Acetobutylicum (SEQ ID NO: 63)

Filtrated broth was adjusted to pH8.5 and filtrated on 0.22 μm PES filter (Nalge Nunc International, Nalgene labware cat#595-4520). The filtrate was added 2% v/v GC-850 (Gulbrandsen, S.C., USA) followed by filtration on 0.22 μm PES filter (Nalge Nunc International, Nalgene labware cat#595-4520). The filtrate was loaded onto a MEP Hypercel™ column (Pall

Corporation, Long Island, N.Y., USA) equilibrated with 50mM TRIS pH8.5. After wash with equilibration buffer, the bound proteins were batch eluted with 100 mM acetic acid pH 4.5. Fractions were collected and analyzed by SDS-PAGE. The fractions were pooled and diluted 4 times with MQ-water. The pH was adjusted to pH6 and applied to SOURCE™ 30S column (GE Healthcare, Piscataway, N.J., USA) equilibrated with 25 mM MES pH 6.0 and bound proteins were eluted with a linear gradient from 0-1000 mM sodium chloride over 10 CV. Fractions were collected and analyzed by SDS-PAGE.

### Example 3

### Cloning of GH30_8 Xylanases (SEQ ID NO: 9, 15, 21, 27, 33, 39, 45, 51 and 57)

Two bacterial GH30 xylanase wild-type sequences were cloned from Pseudoalteromonas tetraodonis (SEQ ID NO: 4) and Paenibacillus sp-19179 (SEQ ID NO: 10).

Bacterial GH30 xylanases were synthesized and purchased commercially based on the nucleotide sequences from Ruminococcus sp. CAG:330 (SEQ ID NO: 22), Streptomyces sp-62627 (SEQ ID NO: 28), Clostridium saccharobutylicum DSM 13864 (SEQ ID NO: 34), Paenibacillus panacisoli (SEQ ID NO: 40), Human Stool metagenome subject 159268001 (SEQ ID NO: 46) or were synthesized and purchased commercially as codon optimized synthetic genes based on the nucleotide sequences from Pectobacterium carotovorum subsp. carotovorum PCC21 (SEQ ID NO: 16) and Vibrio rhizosphaerae DSM 18581 (SEQ ID NO: 52).

The xylanases were cloned into a Bacillus expression vector as described in WO 12/025577. The DNA encoding the mature peptide were cloned in frame to a Bacillus clausii secretion signal (BcSP; with the following amino acid sequence: MKKPLGKIVASTALLISVAFSSSIASA (SEQ ID NO: 2), originating from the protease AprH of B. clausii). BcSP replaced all native secretion signals respectively in all genes.

Downstream of the BcSP sequence an affinity tag sequence was introduced to ease the purification process (His-tag; with the following amino acid sequence: HHHHHHPR (SEQ ID NO: 3) The gene that was expressed therefore comprised the BcSP sequence followed by the His-tag sequence followed by the mature wild type xylanase sequence (as shown in SEQ ID NO: 7, 13, 19, 25, 31, 37, 43, 49 and 55 respectively).

The final expression plasmids (BcSP-His-tag-GH30) were individually transformed into a Bacillus subtilis expression host. The BcSP-fusion genes were integrated by homologous recombination into the Bacillus subtilis host cell genome upon transformation.

The gene construct was expressed under the control of a triple promoter system (as described in WO 99/43835). The gene coding for chloramphenicol acetyltransferase was used as maker (as described in (Diderichsen et al., 1993, Plasmid 30: 312-315)). Transformants were selected on LB media agar supplemented with 6 microgram of chloramphenicol per ml. One recombinant Bacillus subtilis clone containing the respective xylanase expression construct was selected and was cultivated on a rotary shaking table in 500 ml baffled Erlenmeyer flasks each containing 100 ml yeast extract-based media. After 3-5 days cultivation time at 30° C. to 37° C., enzyme containing supernatants were harvested by centrifugation and the enzymes were purified by His-tag purification as described in example 4.

### Example 4

### Purification of GH30_8 Xylanases (SEQ ID NO: 9, 15, 21, 27, 33, 39, 45, 51 and 57)

All His-tagged enzymes were purified by immobilized metal chromatography (IMAC) using Ni²⁺ as the metal ion on 5 mL HisTrap Excel columns (GE Healthcare Life Sciences). The purification took place at pH 8 and the bound proteins were eluted with 50 mM HEPES, pH7.0 and 0.75 M imidazole. Subsequently, the enzyme sample was desalted by loading onto a Sephadex™ G-25 (medium) (GE Healthcare, Piscataway, N.J., USA) column equilibrated in 50 mM HEPES pH 7.0, 100 mM NaCI and eluting with the same buffer. The purity of the purified enzymes was checked by SDS-PAGE and the concentration of each enzyme determined by Abs 280 nm after a buffer exchange.

### Example 5

### Solubilisation of Defatted Destrached Maize using GH30_8 Xylanases

The experiment was performed as described in the Xylose Solubilisation Assay using 20pμm of enzyme and the results are presented in table 3 below. Ronozyme WX, a commercial GH11 xylanase used in animal feed (DSM Nutritional Products) was used as comparison.

TABLE 3
Concentration of xylose and arabinose released after enzymatic hydrolysis¹

| | Xylose | Arabinose | Glucose | Xylan |
|---|---|---|---|---|
| GH30_8 xylanase | (mM) | (mM) | (mM) | solubilization (%) |
| SEQ ID NO: 9 | 1.84 | 2.07 | 0.12 | 13.1 |
| SEQ ID NO: 15 | 1.42 | 1.61 | 0.06 | 10.1 |
| SEQ ID NO: 21 | 0.81 | 0.90 | 0.01 | 5.7 |
| SEQ ID NO: 27 | 1.83 | 2.02 | 0.12 | 13.0 |
| SEQ ID NO: 33 | 0.97 | 1.12 | 0.08 | 6.9 |
| SEQ ID NO: 39 | 1.68 | 1.84 | 0.11 | 12.0 |
| SEQ ID NO: 45 | 2.02 | 2.18 | 0.13 | 14.4 |
| SEQ ID NO: 51 | 1.28 | 1.39 | 0.07 | 9.1 |
| SEQ ID NO: 57 | 1.89 | 2.01 | 0.09 | 13.5 |
| SEQ ID NO: 63 | 1.92 | 2.18 | 0.10 | 13.6 |
| Ronozyme WX | 0.09 | 0.02 | −0.02 | 0.7 |

¹The data is presented after substracting the amount of sugar released from the reference sample in which no enzyme was present.

The results clearly show that a significantly higher amount of xylose and arabinose were released for all GH30 xylanases of the invention compared to the benchmark GH11 xylanase Ronozyme WX. It is also noted that the amount of arabinose released was approximately 1.1 times the amount of xylose released for all GH30 xylanases.

### Example 6

### Solubilisation of Defatted Destrached Maize using GH30 Xylanases

The experiment was performed as described in the Xylose Solubilisation Assay to test the solubilisation ability of a GH30 subfamily 7 xylanase disclosed in WO 2013/067964 (herein assigned as SEQ ID NO: 64). Two doses of the GH30_7 xylanase, 20 ppm and 200 ppm, were used in the assay. For comparision, 20 ppm of SEQ ID NO: 63 (a GH30_8 xylanase of the invention) and 20 ppm of Ronozyme WX (a GH11 xylanase) were used. The results are presented in table 4 below.

TABLE 4
Concentration of xylose and arabinose released after enzymatic hydrolysis¹

| | Enzyme | | | |
|---|---|---|---|---|
| | concentration | Arabinose | Glucose | Xylose |
| Xylanase | (mg EP/kg) | (mM) | (mM) | (mM) |
| SEQ ID NO: 63 | 20 | 2.04 | 0.08 | 1.96 |
| (GH30_8 xylanase) | | | | |
| SEQ ID NO: 64 | 20 | −0.02 | −0.02 | 0.01 |
| (GH30_7 xylanase) | | | | |
| SEQ ID NO: 64 | 200 | −0.02 | 0.02 | 0.01 |
| (GH30_7 xylanase) | | | | |
| Ronozyme WX | 20 | 0.00 | −0.05 | 0.04 |
| (GH11 xylanase) | | | | |
| Buffer | — | −0.02 | −0.01 | −0.01 |

¹The data is presented after substracting the amount of sugar released from the reference sample in which no enzyme was present.

Table 4 shows that SEQ ID NO: 64 did not release any arabinose or xylose, even when a 10 times higher concentration of enzyme was used. The data demonstrates that neither the GH30_7 xylanase nor the GH11 xylanase were able to degrade the highly substituted xylan backdone in DFDSM.

In comparison, the GH30_8 of the invention released a significant amount of arabinose and xylose, thereby demonstrating the ability of this GH30_8 xylanase to degraded the sterically hindered arabinoxylan found in DFDSM.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-25. (canceled)

26. A method of solubilising xylan from plant based material, comprising treating plant based material with a GH30 subfamily 8 polypeptide having xylanase activity, wherein the GH30 subfamily 8 polypeptide having xylanase activity is selected from the group consisting of:
(a) a polypeptide having at least 80% sequence identity to the polypeptide of SEQ ID NO: 60;
(b) a polypeptide having at least 80% sequence identity to the polypeptide of SEQ ID NO: 6;
(c) a polypeptide having at least 92% sequence identity to the polypeptide of SEQ ID NO: 12;
(d) a polypeptide having at least 80% sequence identity to the polypeptide of SEQ ID NO: 18;
(e) a polypeptide having at least 80% sequence identity to the polypeptide of SEQ ID NO: 24;
(f) a polypeptide having at least 80% sequence identity to the polypeptide of SEQ ID NO: 30;
(g) a polypeptide having at least 80% sequence identity to the polypeptide of SEQ ID NO: 36;
(h) a polypeptide having at least 85% sequence identity to the polypeptide of SEQ ID NO: 42;
(i) a polypeptide having at least 80% sequence identity to the polypeptide of SEQ ID NO: 48;
(j) a polypeptide having at least 80% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

27. The method of claim 26, wherein the polypeptide comprises the motif YXVWVY[I/L]RRXYG (SEQ ID NO: 1).

28. The method of claim 26, wherein the polypeptide comprises or consists of amino acids 1 to 557 of SEQ ID NO: 60, amino acids 1 to 569 of SEQ ID NO: 63, amino acids 1 to 382 of SEQ ID NO: 6, amino acids 1 to 390 of SEQ ID NO: 9, amino acids 1 to 391 of SEQ ID NO: 12, amino acids 1 to 399 of SEQ ID NO: 15, amino acids 1 to 383 of SEQ ID NO: 18, amino acids 1 to 391 of SEQ ID NO: 21, amino acids 1 to 565 of SEQ ID NO: 24, amino acids 1 to 573 of SEQ ID NO: 27, amino acids 1 to 396 of SEQ ID NO: 30, amino acids 1 to 404 of SEQ ID NO: 33, amino acids 1 to 392 of SEQ ID NO: 36, amino acids 1 to 400 of SEQ ID NO: 39, amino acids 1 to 413 of SEQ ID NO: 42, amino acids 1 to 421 of SEQ ID NO: 45, amino acids 1 to 398 of SEQ ID NO: 48, amino acids 1 to 406 of SEQ ID NO: 51, amino acids 1 to 382 of SEQ ID NO: 54, amino acids 1 to 390 of SEQ ID NO: 57.

29. The method of claim 26, wherein the percentage of solubilized xylan is at least 4% when the method is performed under the reaction conditions 20 μg GH30 polypeptide per gram defatted destarched maize (DFDSM) and incubation at 40° C., pH 5 for 2.5 hours.

30. The method of claim 26, wherein the plant based material is from the sub-family Panicoideae.

31. A granule comprising one or more GH30 subfamily 8 polypeptides having xylanase activity, wherein the GH30 subfamily 8 polypeptide having xylanase activity is selected from the group consisting of:
(a) a polypeptide having at least 80% sequence identity to the polypeptide of SEQ ID NO: 60;
(b) a polypeptide having at least 80% sequence identity to the polypeptide of SEQ ID NO: 6;
(c) a polypeptide having at least 92% sequence identity to the polypeptide of SEQ ID NO: 12;
(d) a polypeptide having at least 80% sequence identity to the polypeptide of SEQ ID NO: 18;
(e) a polypeptide having at least 80% sequence identity to the polypeptide of SEQ ID NO: 24;
(f) a polypeptide having at least 80% sequence identity to the polypeptide of SEQ ID NO: 30;
(g) a polypeptide having at least 80% sequence identity to the polypeptide of SEQ ID NO: 36;
(h) a polypeptide having at least 85% sequence identity to the polypeptide of SEQ ID NO: 42;
(i) a polypeptide having at least 80% sequence identity to the polypeptide of SEQ ID NO: 48;
(j) a polypeptide having at least 80% sequence identity to the polypeptide of SEQ ID NO: 54;
(k) a variant of the polypeptide of SEQ ID NO: 60, SEQ ID NO: 6, SEQ ID NO: 18, SEQ ID NO: 24, SEQ ID NO: 30, SEQ ID NO: 36, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(l) a variant of the polypeptide of SEQ ID NO: 12 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(m) a polypeptide comprising the polypeptide of (a), (b), (c), (d), (e), (f), (g), (h), (i), (j), (k) or (l) and a N-terminal and/or C-terminal His-tag and/or HQ-tag; and
(n) a fragment of a polypeptide of (a), (b), (c), (d), (e), (f), (g), (h), (i), (j), (k), (l) or (m) having at least 90% of the length of the mature polypeptide.

32. The granule of claim 31, wherein the polypeptide comprises the motif YXVWVY[I/L]RRXYG (SEQ ID NO: 1).

33. The granule of claim 31, wherein the granule comprises one or more formulating agents.

34. The granule of claim 31, wherein the granule comprises a core particle and one or more coatings.

35. The granule of claim 31, wherein the polypeptide solubilizes at least 4% xylan from defatted destarched maize (DFDSM).

36. An isolated polypeptide having xylanase activity, selected from the group consisting of:
(a) a polypeptide having at least 80% sequence identity to the polypeptide of SEQ ID NO: 6;
(b) a polypeptide having at least 92% sequence identity to the polypeptide of SEQ ID NO: 12;
(c) a polypeptide having at least 86% sequence identity to the polypeptide of SEQ ID NO: 30;
(d) a polypeptide having at least 90% sequence identity to the polypeptide of SEQ ID NO: 36;
(e) a polypeptide having at least 85% sequence identity to the polypeptide of SEQ ID NO: 42;
(f) a polypeptide having at least 80% sequence identity to the polypeptide of SEQ ID NO: 48;
(g) a polypeptide having at least 80% sequence identity to the polypeptide of SEQ ID NO: 54;
(h) a polypeptide encoded by a polynucleotide that hybridizes under high stringency conditions, or very high stringency conditions with
(i) the mature polypeptide coding sequence of SEQ ID NO: 4,
(ii) the mature polypeptide coding sequence of SEQ ID NO: 10,
(iii) the mature polypeptide coding sequence of SEQ ID NO: 28,
(iv) the mature polypeptide coding sequence of SEQ ID NO: 34,
(v) the mature polypeptide coding sequence of SEQ ID NO: 40,
(vi) the mature polypeptide coding sequence of SEQ ID NO: 46,
(vii) the mature polypeptide coding sequence of SEQ ID NO: 82,
(viii) the full-length complementary strand of (i), (ii), (iii), (iv), (v), (vi) or (vii);
(i) a polypeptide encoded by a polynucleotide having at least 80% sequence identity to the mature polypeptide coding sequence of SEQ ID NO: 4;
(j) a polypeptide encoded by a polynucleotide having at least 90% sequence identity to the mature polypeptide coding sequence of SEQ ID NO: 10;
(k) a polypeptide encoded by a polynucleotide having at least 86% sequence identity to the mature polypeptide coding sequence of SEQ ID NO: 28;
(l) a polypeptide encoded by a polynucleotide having at least 90% sequence identity to the mature polypeptide coding sequence of SEQ ID NO: 34;
(m) a polypeptide encoded by a polynucleotide having at least 85% sequence identity to the mature polypeptide coding sequence of SEQ ID NO: 40;
(n) a polypeptide encoded by a polynucleotide having at least 80% sequence identity to the mature polypeptide coding sequence of SEQ ID NO: 46;
(o) a polypeptide encoded by a polynucleotide having at least 80% sequence identity to the mature polypeptide coding sequence of SEQ ID NO: 52;
(p) a variant of the polypeptide of SEQ ID NO: 6, SEQ ID NO: 18, SEQ ID NO: 30, SEQ ID NO: 42, SEQ ID NO: 48 or SEQ ID NO: 54 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(q) a variant of the polypeptide of SEQ ID NO: 12 or SEQ ID NO: 36 comprising one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 positions;
(r) a polypeptide comprising the polypeptide of (a), (b), (c), (d), (e), (f), (g), (h), (i), (j), (k), (l), (m), (n), (o), (p) or (q) and a N-terminal and/or C-terminal His-tag and/or HQ-tag; and
(s) a fragment of a polypeptide of (a), (b), (c), (d), (e), (f), (g), (h), (i), (j), (k), (l), (m), (n), (o), (p), (q) or (r) having xylanase activity and having at least 90% of the length of the mature polypeptide.

37. A composition comprising the polypeptide of claim 36.

38. An animal feed additive comprising a GH30 subfamily 8 polypeptide of claim 36 and one or more items selected from the group consisting of vitamins and minerals.

39. An animal feed comprising the granule of claim 31, the polypeptide of claim 36, the composition of claim 37, or the animal feed additive of claim 38 and plant based material from the sub-family Panicoideae.

40. A method of improving one or more performance parameters of an animal comprising administering to one or more animals the granule of claim 31, the polypeptide of claim 36, the composition of claim 37, or the animal feed additive of claim 38.

41. A method of preparing an animal feed comprising mixing the granule of claim 31, the polypeptide of claim 36, the composition of claim 37, or the animal feed additive of claim 38 with plant based material from the sub-family Panicoideae.

42. A method for improving the nutritional value of an animal feed comprising plant based material from the sub-family Panicoideae, comprising adding to the feed the granule of claim 31, the polypeptide of claim 36, the composition of claim 37, or the animal feed additive of claim 38.

43. A nucleic acid construct or expression vector comprising a polynucleotide encoding the polypeptide of claim 36, wherein the polynucleotide is operably linked to one or more control sequences that direct the production of the polypeptide in an expression host.

44. A recombinant host cell comprising the polynucleotide encoding the polypeptide of claim 36, wherein the polynucleotide is operably linked to one or more control sequences that direct the production of the polypeptide.

45. A method of producing a polypeptide having xylanase activity, comprising:
(a) cultivating a host cell of claim 44 under conditions conducive for production of the polypeptide; and
(b) recovering the polypeptide.
